SEQUENCE LISTING (10 sequences)

SEQ ID NO: 1 - protein, 374 aa, Artificial sequence
  Synthetic
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLE

SEQ ID NO: 2 - protein, 353 aa, Artificial sequence
  Synthetic
RPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLE

SEQ ID NO: 3 - protein, 360 aa, Artificial sequence
  Synthetic
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEAL

SEQ ID NO: 4 - protein, 339 aa, Artificial sequence
  Synthetic
RPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEAL

SEQ ID NO: 5 - protein, 592 aa, Artificial sequence
  Synthetic
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 571 aa, Artificial sequence
  Synthetic
RPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 21 aa, Artificial sequence
  Synthetic
MWSWKCLLFWAVLVTATLCTA

SEQ ID NO: 8 - protein, 232 aa, Artificial sequence
  Synthetic
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 228 aa, Artificial sequence
  Synthetic
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 229 aa, Artificial sequence
  Synthetic
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2016/0067307 A1
(43) Pub. Date: Mar. 10, 2016

(54) METHODS OF TREATING CANCER

(71) Applicants: FIVE PRIME THERAPEUTICS, INC., South San Francisco, CA (US); GLAXOSMITHKLINE INTELLECTUAL PROPERTY (NO.2) LIMITED, Middlesex (GB)

(72) Inventors: Julie Hambleton, San Francisco, CA (US); Maureen R. Bleam, Upper Providence, PA (US); Maurice P. DeYoung, Upper Providence, PA (US); Geraldine Ferron-Brady, Upper Providence, PA (US); Rakesh Kumar, Upper Providence, PA (US); Lone Ottesen, Stockley Park (UK)

(21) Appl. No.: 14/785,691

(22) PCT Filed: Apr. 30, 2014
(86) PCT No.: PCT/US2014/036140

§ 371 Date: Oct. 20, 2015

Related U.S. Application Data
(60) Provisional application No. 61/818,220, filed on May 1, 2013.
(60) Provisional application No. 61/831,029, filed on Jun. 4, 2013.

(51) Int. Cl. A61K 38/17

(52) U.S. Cl. CPC A61K 38/179

(57) ABSTRACT

Methods of treating cancers comprising FGFR1 gene amplification, FGFR1 overexpression, FGFR3 overexpression, FGFR3 amplification, FGF2 overexpression, and/or FGF2 gene amplification are provided. In some embodiments, the methods comprise administering a fibroblast growth factor receptor 1 (FGFR1) extracellular domain (ECD) and/or an FGFR1 ECD fusion molecule. In some embodiments, the methods comprise administering a FGFR1 ECD and/or an FGFR1 ECD fusion molecule in combination with at least one additional therapeutic agent. In some embodiments, methods of treating cancers comprising administering a FGFR1 ECD and/or an FGFR1 ECD fusion molecule in combination with at least one chemotherapeutic agent are provided.

### BACKGROUND

Soluble forms of Fibroblast Growth Factor Receptor 1 (FGFR1) have been shown to inhibit tumor cell growth in vitro and in vivo. See, e.g., U.S. Pat. No. 7,678,890. The efficacy of anti-cancer therapies is, in some instances, dependent on the genetic makeup of the cancer being targeted.

### SUMMARY OF THE INVENTION

In some embodiments, methods of treating breast cancer having FGFR1 gene amplification, FGFR1 overexpression, FGFR3 gene amplification, FGFR3 overexpression, FGF2 gene amplification and/or FGF2 overexpression are provided, comprising administering to the subject a therapeutically effective amount of a fibroblast growth factor receptor 1 (FGFR1) extracellular domain (ECD) or an FGFR1 ECD fusion molecule. In some embodiments, the breast cancer has been determined to be estrogen receptor (ER) positive, progesterone (PR) positive, or ER positive and PR positive. In some embodiments, the breast cancer has been determined to be ER positive. In some embodiments, the breast cancer has been determined to be PR positive. In some embodiments, the breast cancer has been determined to be ER positive and PR positive. In some embodiments, the breast cancer has been determined to be HER2 positive. In some embodiments, the breast cancer has been determind to be p95HER2 positive. In some embodiments, the breast cancer has been determined to be HER2 negative. In any of the embodiments described herein, the breast cancer may be metastatic breast cancer. In any of the embodiments described herein, the subject with breast cancer is post-menopausal.

In some embodiments, the subject with breast cancer has previously been administered, or is currently being administered, trastuzumab (e.g., Herceptin®) and/or lapatinib (e.g., Tykerb®). In some embodiments, the subject has previously been administered, or is currently being administered, an aromatase inhibitor. In some embodiments, the aromatase inhibitor is selected from aminoglutethimide, testolactone (e.g., Teslac®), anastrozole (e.g., Arimidex®), letrozole (e.g., Femara®), exemestane (e.g., Aromasin®), vorozole (e.g., Rivisor®), formestane (e.g., Lentaron®), megestrol acetate (e.g., Megase®), and fadrozole (e.g., Afema®). In some embodiments, a subject with breast cancer has previously been administered, or is currently being administered, an ER antagonist. In some embodiments, the subject has previously been determined to have ER positive breast cancer. Nonlimiting exemplary ER antagonists include tamoxifen (e.g., Nolvadex®, Istubal®, and Valodex®) and fulvestrant (e.g., Faslodex®).

In some embodiments, methods of treating prostate cancer having FGFR1 gene amplification, FGFR1 overexpression, FGFR3 gene amplification, FGFR3 overexpression, FGF2 gene amplification and/or FGF2 overexpression are provided, comprising administering to the subject a therapeutically effective amount of a fibroblast growth factor receptor 1 (FGFR1) extracellular domain (ECD) or an FGFR1 ECD fusion molecule. In some embodiments, the subject has previously been administered, or is currently being administered, a therapeutic agent selected from a gonadotropin releasing hormone (GnRH) agonist, a GnRH antagonist, an androgen receptor (AR) inhibitor, a 17-hydroxylase inhibitor, and diethylstilbestrol (DES). In some embodiments, the subject has previously been administered, or is currently being administered, a gonadotropin releasing hormone (GnRH) agonist or a GnRH antagonist. In some embodiments, the subject has previously been administered, or is currently being administered, a GnRH antagonist. In some embodiments, the GnRH agonist is selected from leuprolide (e.g., Lupron®, Eligard®), buserelin (e.g., Suprefact®, Suprecor®), histrelin (e.g., Supprelin LA®, Vantas®), goserelin acetate (e.g., Zoladex®), deslorelin (e.g., Suprelorin®, Ovuplant®), nafarelin (e.g., Synarel®), and triptorelin. In some embodiments, the GnRH antagonist is selected from cetrorelix (e.g., Cetrotide®), ganirelix (e.g., Antagon®), abarelix (e.g., Plenaxis®), and degarelix (e.g., Firmagon®). In some embodiments, an AR inhibitor is selected from cyproterone acetate (e.g., Androcur®, Cyprostat®), flutamide (e.g., Eulexin®), bicalutamide (e.g., Casodex®), enzalutamide (e.g., Xtandi®), ketoconazole, and nilutamide (e.g., Anandron®, Nilandron®). In some embodiments, a 17-hydroxylase inhibitor is abiraterone acetate (e.g., Zytiga®).

In some embodiments, methods of treating carcinoid cancer having FGFR1 gene amplification, FGFR1 overexpression, FGFR3 gene amplification, FGFR3 overexpression, FGF2 gene amplification and/or FGF2 overexpression are provided, comprising administering to the subject a therapeutically effective amount of a fibroblast growth factor receptor 1 (FGFR1) extracellular domain (ECD) or an FGFR1 ECD fusion molecule. In some embodiments, the subject has previously been administered, or is currently being administered, octreotide. In some embodiments, therapeutically effective amount of octreotide has been previously administered, or is currently being administered to the subject.

In some embodiments, methods of treating ovarian cancer having FGFR1 gene amplification, FGFR1 overexpression, FGFR3 gene amplification, FGFR3 overexpression, FGF2 gene amplification and/or FGF2 overexpression are provided, comprising administering to the subject a therapeutically effective amount of a fibroblast growth factor receptor 1 (FGFR1) extracellular domain (ECD) or an FGFR1 ECD fusion molecule. In some embodiments, the subject has previously been administered, or is currently being administered, an ER antagonist or an aromatase inhibitor. In some embodiments, the aromatase inhibitor is selected from aminoglutethimide, testolactone (e.g., Teslac®), anastrozole (e.g., Arimidex®), letrozole (e.g., Femara®), exemestane (e.g., Aromasin®), vorozole (e.g., Rivisor®), formestane (e.g., Lentaron®), megestrol acetate (e.g., Megase®), and fadrozole (e.g., Afema®). Nonlimiting exemplary ER antagonists include tamoxifen (e.g., Nolvadex®, Istabul®, Valodex®) and fulvestrant (e.g., Faslodex®). In some embodiments, the ovarian cancer is estrogen receptor (ER) positive, progesterone (PR) positive, or ER positive and PR positive.

In some embodiments, methods of treating lung cancer in a subject are provided. In some embodiments, a method comprises administering at least 5 mg/kg of an FGFR1 ECD or an FGFR1 ECD fusion molecule and at least 135 mg/m² paclitaxel and at least AUC 4 carboplatin to the subject. In some embodiments, the method comprises administering from 135 mg/m² paclitaxel to 200 mg/m² paclitaxel, at least 175 mg/m² paclitaxel, from 175 mg/m² paclitaxel to 200 mg/m² paclitaxel, or 200 mg/m² paclitaxel. In some embodiments, the method comprises administering from AUC 4 carboplatin to AUC 6 carboplatin, at least AUC 5 carboplatin, from AUC 5 carboplatin to AUC 6 carboplatin, or AUC 6 carboplatin. In some embodiments, the lung cancer is non-small cell lung cancer. In some embodiments, the non-small cell lung cancer is squamous non-small cell lung cancer.

In some embodiments, a method of treating lung cancer in a subject comprises administering at least 5 mg/kg of an FGFR1 ECD or an FGFR1 ECD fusion molecule and at least 40 mg/m² docetaxel. In some embodiments, the method comprises administering from 40 mg/m² docetaxel to 75 mg/m² docetaxel, at least 55 mg/m² docetaxel, from 55 mg/m² docetaxel to 75 mg/m² docetaxel, or 75 mg/m² docetaxel. In some embodiments, the lung cancer is non-small cell lung cancer. In some embodiments, the non-small cell lung cancer is squamous non-small cell lung cancer.

In any of the embodiments described herein, a method may comprise administering from 5 mg/kg to 20 mg/kg of an FGFR1 ECD or an FGFR1 ECD fusion molecule, at least 10 mg/kg of an FGFR1 ECD or an FGFR1 ECD fusion molecule, from 10 mg/kg to 20 mg/kg of an FGFR1 ECD or an FGFR1 ECD fusion molecule, at least 15 mg/kg of an FGFR1 ECD or an FGFR1 ECD fusion molecule, from 15 mg/kg to 20 mg/kg of an FGFR1 ECD or an FGFR1 ECD fusion molecule, or 20 mg/kg of an FGFR1 ECD or an FGFR1 ECD fusion molecule.

In any of the embodiments described herein, at least a portion of the cancer cells may have an FGFR1 gene amplification. In some embodiments, at least a portion of the cells of the cancer comprise at least three, at least four, at least five, at least six, at least eight, or at least ten copies of the FGFR1 gene. In some embodiments, at least a portion of the cells of the cancer have a ratio of FGFR1 gene to chromosome 8 centromere of at least 1.5, at least 2, at least 2.5, at least 3, at least 3.5, or at least 4. In some embodiments, at least a portion of the cells of the cancer have a ratio of FGFR1 gene to chromosome 8 centromere of greater than 2.

In any of the embodiments described herein, at least a portion of the cancer cells may have an FGFR3 gene amplification. In some embodiments, at least a portion of the cells of the cancer comprise at least three, at least four, at least five, at least six, at least eight, or at least ten copies of the FGFR3 gene. In some embodiments, at least a portion of the cells of the cancer have a ratio of FGFR3 gene to chromosome 4 centromere of at least 1.5, at least 2, at least 2.5, at least 3, at least 3.5, or at least 4. In some embodiments, at least a portion of the cells of the cancer have a ratio of FGFR3 gene to chromosome 4 centromere of greater than 2.

In any of the embodiments described herein, at least a portion of the cancer cells may have an FGF2 gene amplification. In some embodiments, at least a portion of the cells of the cancer comprise at least three, at least four, at least five, at least six, at least eight, or at least ten copies of the FGF2 gene. In some embodiments, at least a portion of the cells of the cancer have a ratio of FGF2 gene to chromosome 4 centromere of at least 1.5, at least 2, at least 2.5, at least 3, at least 3.5, or at least 4. In some embodiments, at least a portion of the cells of the cancer have a ratio of FGF2 gene to chromosome 4 centromere of greater than 2.

In any of the embodiments described herein, gene amplification may be determined by a method selected from fluorescence in situ hybridization, array comparative genomic hybridization, DNA microarray, spectral karyotyping, quantitative PCR, southern blotting, or sequencing.

In any of the embodiments described herein, at least a portion of the cells of the cancer may have FGFR1 overexpression. In some embodiments, FGFR1 is FGFR1IIIc. In any of the embodiments described herein, at least a portion of the cells of the cancer may have FGF2 overexpression. In any of the embodiments described herein, at least a portion of the cells of the cancer may have FGFR3 overexpression. In some embodiments, FGFR3 is FGFR3IIIc. In any of the embodiments described herein, at least a portion of the cells of the cancer may overexpress at least one, at least two, or three markers selected from DKK3, FGF18, and ETV4. In any of the embodiments described herein, at least a portion of the cells of the cancer may overexpress at least one or two markers selected from DKK3 and FGF18. In any of the embodiments described herein, at least a portion of the cells of the cancer may overexpress ETV4. In some embodiments, the cancer does not have FGFR1 gene amplification.

In some embodiments, the overexpression is mRNA overexpression. In some embodiments, mRNA overexpression is determined using quantitative RT-PCR. In some embodiments, the overexpression is protein overexpression. In some embodiments, protein overexpression is determined using immunohistochemistry.

In any of the embodiments described herein, the method may comprise administering an FGFR1 ECD. In some such embodiments, the FGFR1 ECD comprises an amino acid sequence selected from SEQ ID NOs: 1 to 4. In any of the embodiments described herein, the method may comprise administering an FGFR1 ECD fusion molecule, wherein the FGFR1 ECD fusion molecule comprises an FGFR1 ECD and at least one fusion partner. In some embodiments, at least one fusion partner is selected from an Fc, albumin, and polyethylene glycol. In some embodiments, at least one fusion partner is an Fc. In some embodiments, the Fc comprises an amino acid sequence selected from SEQ ID NOs: 8 to 10. In some embodiments, the FGFR1 ECD fusion molecule comprises a sequence selected from SEQ ID NO: 5 and SEQ ID NO: 6. In some embodiments, the at least one fusion partner is an Fc and polyethylene glycol. In some embodiments, the at least one fusion partners is polyethylene glycol. In some embodiments, the fusion molecule comprises a linker between the FGFR1 ECD and one or more fusion partners. In some embodiments, the FGFR1 ECD fusion molecule is FGFR1 ECD.339-Fc.

In some embodiments, an FGFR1 ECD or FGFR1 ECD fusion molecule is glycosylated and/or sialylated. In some embodiments, an FGFR1 ECD or the polypeptide portion of the FGFR1 ECD fusion molecule is expressed in Chinese hamster ovary (CHO) cells. In some embodiments, an FGFR1 ECD comprises an amino acid sequence selected from SEQ ID NO: 1 and SEQ ID NO: 3.

In some embodiments, the FGFR1 ECD or FGFR1 ECD fusion molecule is an amount in the range of about 0.5 mg/kg body weight to about 30 mg/kg body weight, such as an amount in the range of about 5 to about 20 mg/kg body weight (e.g., using an EC=1.11 mL/mg*cm, as shown in Table 1). In some embodiments, the therapeutically effective amount of the FGFR1 ECD or FGFR1 ECD fusion molecule is a dose of about 5 mg/kg body weight. In some embodiments, the therapeutically effective amount of the FGFR1 ECD or FGFR1 ECD fusion molecule is a dose of about 10 mg/kg body weight. In some embodiments, the therapeutically effective amount of the FGFR1 ECD or FGFR1 ECD fusion molecule is a dose of about 15 mg/kg body weight. In some embodiments, the therapeutically effective amount of the FGFR1 ECD or FGFR1 ECD fusion molecule is a dose of about 20 mg/kg body weight. In some embodiments, dosages may be administered twice a week, weekly, every other week, at a frequency between weekly and every other week, every three weeks, every four weeks, or every month.

In some embodiments, a method described herein further comprises administering at least one additional therapeutic agent. In some embodiments, at least one additional therapeutic agent is an anti-cancer agent. In some embodiments, at least one additional therapeutic agent is a chemotherapeutic agent. at least one additional therapeutic agent is an anti-angiogenic agent. Nonlimiting exemplary anti-cancer agents, chemotherapeutic agents, and anti-angiogenic agents are described herein.

In some embodiments, methods of identifying a subject with breast cancer who may benefit from administration of an FGFR1 ECD or FGFR1 ECD fusion molecule are provided. In some embodiments, a method comprises determining whether at least a portion of the cancer cells in a sample obtained from the subject comprise FGFR1 gene amplification, FGFR1 overexpression, FGFR3 gene amplification, FGFR3 overexpression, FGF2 gene amplification and/or FGF2 overexpression; and determining whether the cancer is estrogen receptor (ER) positive, progesterone (PR) positive, or ER positive and PR positive. In some embodiments, if a cancer has FGFR1 gene amplification, FGFR1 overexpression, FGFR3 gene amplification, FGFR3 overexpression, FGF2 gene amplification and/or FGF2 overexpression; and the cancer is ER positive and/or PR positive, the cancer is predicted to be responsive to an FGFR1 ECD or FGFR1 ECD fusion molecule.

In some embodiments, methods of identifying a subject with cancer who may benefit from administration of an FGFR1 ECD or FGFR1 ECD fusion molecule are provided. In some embodiments, a method comprises determining whether at least a portion of the cancer cells in a sample obtained from the subject overexpress at least one, at least two, at least three, at least four, or at least five markers selected from FGFR1, FGFR3IIIc, FGF2, DKK3, FGF18, and ETV4, wherein overexpression is indicative of therapeutic responsiveness by the cancer to an FGFR1 ECD or FGFR1 ECD fusion molecule. In some embodiments, the method comprises determining whether at least a portion of the cancer cells in a sample obtained from the subject overexpress at least one, at least two, at least three, or at least four markers selected from FGFR1, FGFR3IIIc, FGF2, DKK3, and FGF18. In some embodiments, the method comprises determining whether at least a portion of the cancer cells in a sample obtained from the subject overexpress ETV4. In some embodiments, including any of the foregoing embodiments, the method comprises determining whether at least a portion of the cancer cells in a sample obtained from the subject overexpress Gene 1 and Gene 2 from any line in Table 6 below, or any combination thereof. In some embodiments, FGFR1 is FGFR1IIIc. In some embodiments, including any of the foregoing embodiments, the method comprises determining whether at least a portion of the cancer cells in a sample obtained from the subject have an FGFR1 gene amplification.

Any embodiment described herein or any combination thereof applies to any and all methods of the invention described herein.

### BRIEF DESCRIPTION OF THE DRAWINGS

FIG. 1 shows % tumor growth inhibition by FGFR1-ECD.339-Fc in mouse xenografts of tumor cells having FGFR1 gene amplification and tumor cells having a non-amplified FGFR1 gene, as described in Example 1.

FIG. 2 shows a scatter plot of FGFR1 mRNA expression in lung cancer cell lines with and without FGFR1 gene amplification, as described in Example 2.

FIG. 3 shows graphs of (A) average luminescence in the CellTiterGlo® assay and (B) counts per minute in the tritiated thymidine incorporation assay carried out on NCI-H226 cells grown with varying amounts of serum and in the presence or absence of FGFR1-ECD.339-Fc, as described in Example 2.

FIG. 4 shows a scatter plot of FGFR1 mRNA expression in lung cancer xenografts with and without FGFR1 gene amplification, as described in Example 2.

FIG. 5 shows mean tumor volume at various time points in mice implanted with PDX D35087 cells and treated with FGFR1-ECD.339-Fc or albumin, as described in Example 2.

FIG. 6 shows (A) FGF2 mRNA (normalized to GUSB) and (B) FGF2 protein expression (normalized to total protein) in FGFR1-ECD.339-Fc responder and non-responder xenografts, as described in Example 3.

FIG. 7 shows DKK3 mRNA expression (normalized to GUSB) in FGFR1-ECD.339-Fc responder and non-responder xenografts, as described in Example 4.

FIG. 8 shows anti-tumor activity of FGFR1-ECD.339-Fc in (A) a Caki-1 renal cell carcinoma xenograft model, and (B) a MSTO-211H mesothelioma xenograft model, as described in Example 3.

FIG. 9 shows (A) FGFR1 and (B) FGFR3IIIc mRNA expression in FGFR1-ECD.339-Fc responsive and non-responsive xenograft models, as described in Example 3.

FIG. 10 shows FGFR1-ECD.339-Fc mediated inhibition of FGF-2 and VEGF-A induced angiogenesis in a matrigel plug assay, as described in Example 5.

FIG. 11 shows that FGFR1-ECD.339-Fc does not inhibit VEGF-A induced human umbilical vein endothelial cell (HUVEC) proliferation, as described in Example 5.

FIG. 12 shows FGFR1-ECD.339-Fc mediated inhibition of FGFR1 signaling in a JIMT-1 breast cancer xenograft, as described in Example 6.

### DETAILED DESCRIPTION

The section headings used herein are for organizational purposes only and are not to be construed as limiting the subject matter described.

### DEFINITIONS

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

Unless specifically indicated otherwise, all chemical entities recited herein are intented to include pharmaceutically acceptable forms thereof. Pharmaceutically acceptable forms of the chemical entities recited herein include pharmaceutically acceptable salts, solvates, crystal forms (including polymorphs and clathrates), chelates, non-covalent complexes, prodrugs, and mixtures thereof.

Certain techniques used in connection with recombinant DNA, oligonucleotide synthesis, tissue culture and transformation (e.g., electroporation, lipofection), enzymatic reactions, and purification techniques are known in the art. Many such techniques and procedures are described, e.g., in Sambrook et al. Molecular Cloning: A Laboratory Manual (2nd ed., Cold Spring Harbor Laboratory Press, Cold Spring Harbor, N.Y. (1989)), among other places. In addition, certain techniques for chemical syntheses, chemical analyses, pharmaceutical preparation, formulation, and delivery, and treatment of patients are also known in the art.

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

As used herein, all numbers are approximate, and may be varied to account for measurement error and the rounding of significant digits. The use of “about” before certain measured quantities includes variations due to sample impurities, measurement error, human error, and statistical variation, as well as the rounding of significant digits.

As utilized in accordance with the present disclosure, the following terms, unless otherwise indicated, shall be understood to have the following meanings:

The terms “nucleic acid molecule” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification. When a polypeptide “consists of” a particular amino acid sequence, it may still contain post-translational modifications, such as glycosylation and sialylation.

The term “FGFR1 extracellular domain” (“FGFR1 ECD”) includes full-length FGFR1 ECDs, FGFR1 ECD fragments, and FGFR1 ECD variants. As used herein, the term “FGFR1 ECD” refers to an FGFR1 polypeptide that lacks the intracellular and transmembrane domains, with or without a signal peptide. In some embodiment, the FGFR1 ECD is a human full-length FGFR1 ECD having an amino acid sequence selected from SEQ ID NOs: 1 and 2. The term “full-length FGFR1 ECD”, as used herein, refers to an FGFR1 ECD that extends to the last amino acid of the extracellular domain, and may or may not include an N-terminal signal peptide. As defined herein, the last amino acid of the full-length FGFR1 ECD is at position 353. Thus, a human full-length FGFR1 ECD may consist of the amino acid sequence corresponding to SEQ ID NO.: 2 (mature form) or to SEQ ID NO.: 1 (with the signal peptide). As used herein, the term “FGFR1 ECD fragment” refers to an FGFR1 ECD having one or more residues deleted from the N and/or C terminus of the full-length ECD and that retains the ability to bind to FGF-2. The FGFR1 ECD fragment may or may not include an N-terminal signal peptide. In some embodiments, the FGFR1 ECD fragment is a human FGFR1 ECD fragment having an amino acid sequence corresponding to SEQ ID NO.: 4 (mature form) or to SEQ ID NO.: 3 (with the signal peptide).

As used herein, the term “FGFR1 ECD variants” refers to FGFR1 ECDs that contain amino acid additions, deletions, and substitutions and that remain capable of binding to FGF-2. Such variants may be at least 90%, 92%, 95%, 97%, 98%, or 99% identical to the parent FGFR1 ECD. The % identity of two polypeptides can be measured by a similarity score determined by comparing the amino acid sequences of the two polypeptides using the Bestfit program with the default settings for determining similarity. Bestfit uses the local homology algorithm of Smith and Waterman, Advances in Applied Mathematics 2:482-489 (1981) to find the best segment of similarity between two sequences. In some embodiments, an FGFR1 ECD variant is at least 95% identical to the sequence of SEQ ID NO: 4.

A polypeptide having an amino acid sequence at least, for example, 95% identical to a reference amino acid sequence of an FGFR1 ECD polypeptide is one in which the amino acid sequence of the polypeptide is identical to the reference sequence except that the polypeptide sequence may include up to five amino acid alterations per each 100 amino acids of the reference polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids, up to 5% of the total amino acid residues in the reference sequence, may be inserted into the reference sequence. These alterations of the reference sequence may occur at the N- or C-terminal positions of the reference amino acid sequence or anywhere between those terminal positions, interspersed either individually among residues in the reference sequence, or in one or more contiguous groups within the reference sequence.

As a practical matter, whether any particular polypeptide is at least 70%, 80%, 90%, or 95% identical to, for instance, an amino acid sequence or to a polypeptide sequence encoded by a nucleic acid sequence set forth in the Sequence Listing can be determined conventionally using known computer programs, such the Bestfit program. When using Bestfit or other sequence alignment program to determine whether a particular sequence is, for instance, 95% identical to a reference sequence according to the present invention, the parameters are set, of course, that the percentage of identity is calculated over the full length of the reference amino acid sequence and that gaps in homology of up to 5% of the total number of amino acid residues in the reference sequence are allowed.

As used herein, the terms “hFGFR1-ECD.353” and “hFGFR1.353” may be used interchangeably to refer to the full-length human FGFR1 ECD corresponding to SEQ ID NO: 1 (with signal peptide) or to SEQ ID NO: 2 (without signal peptide; mature form).

As used herein, the terms “hFGFR1-ECD.339” and “hFGFR1.339” may be used interchangeably to refer to the human FGFR1 ECD corresponding to SEQ ID NO: 3 (with signal peptide) or to SEQ ID NO: 4 (without signal peptide; mature form).

Additional hFGFR1 ECDs are described, for example, in U.S. Pat. No. 7,678,890, which is incorporated by reference herein in its entirety for any purpose.

The term “FGFR1 ECD fusion molecule” refers to a molecule comprising an FGFR1 ECD, and one or more “fusion partners.” In some embodiments, the FGFR1 ECD and the fusion partner are covalently linked (“fused”). If the fusion partner is also a polypeptide (“the fusion partner polypeptide”), the FGFR1 ECD and the fusion partner polypeptide may be part of a continuous amino acid sequence, and the fusion partner polypeptide may be linked to either the N terminus or the C terminus of the FGFR1 ECD. In such cases, the FGFR1 ECD and the fusion partner polypeptide may be translated as a single polypeptide from a coding sequence that encodes both the FGFR1 ECD and the fusion partner polypeptide (the “FGFR1 ECD fusion protein”). In some embodiments, the FGFR1 ECD and the fusion partner are covalently linked through other means, such as, for example, a chemical linkage other than a peptide bond. Many known methods of covalently linking polypeptides to other molecules (for example, fusion partners) may be used. In other embodiments, the FGFR1 ECD and the fusion partner may be fused through a “linker,” which is comprised of at least one amino acid or chemical moiety.

In some embodiments, the FGFR1 ECD polypeptide and the fusion partner are noncovalently linked. In some such embodiments, they may be linked, for example, using binding pairs. Exemplary binding pairs include, but are not limited to, biotin and avidin or streptavidin, an antibody and its antigen, etc.

Exemplary fusion partners include, but are not limited to, an immunoglobulin Fc domain, albumin, and polyethylene glycol. The amino acid sequences of some exemplary Fc domains are shown in SEQ ID NOs: 8 to 10. In some embodiments, an FGFR1 ECD fused to an Fc is referred to as an “hFGFR1 ECD-Fc.” In some embodiments, the Fc domain is selected from an IgG1 Fc, an IgG2 Fc, an IgG3 Fc, and an IgG4 Fc.

As used herein, the terms “hFGFR1-ECD.339-Fc” and “hFGFR1.339-Fc” may be used interchangeably to refer to an amino acid sequence selected from SEQ ID NO: 6 (without signal peptide, mature form) and SEQ ID NO: 5 (with signal peptide). Nonlimiting exemplary cancers that may be treated with hFGFR1-ECD.339-Fc include, but are not limited to, lung cancer, colon cancer, breast cancer, gastric cancer, head and neck cancer, prostate cancer, endometrial cancer, sarcoma, small cell lung cancer, ovarian cancer, Kaposi's sarcoma, Hodgkin's disease, leukemia, non-Hodgkin's lymphoma, neuroblastoma (brain cancer), rhabdomyosarcoma, Wilms' tumor, acute lymphoblastic leukemia, acute lymphoblastic leukemia, bladder cancer, testicular cancer, lymphomas, germ cell tumors, cancers of the colon and rectum, gastrointestinal cancers, thyroid cancer, multiple myeloma, pancreatic cancer, mesothelioma, malignant pleural mesothelioma, hematological/lymphatic cancers, malignant peritoneal mesothelioma, esophageal cancer, renal cell carcinoma, glioblastoma multiforme, and liver cancer.

The term “signal peptide” refers to a sequence of amino acid residues located at the N terminus of a polypeptide that facilitates secretion of a polypeptide from a mammalian cell. A signal peptide may be cleaved upon export of the polypeptide from the mammalian cell, forming a mature protein. Signal peptides may be natural or synthetic, and they may be heterologous or homologous to the protein to which they are attached. Exemplary signal peptides include, but are not limited to, FGFR1 signal peptides, such as, for example, the amino acid sequence of SEQ ID NO: 7. Exemplary signal peptides also include signal peptides from heterologous proteins. A “signal sequence” refers to a polynucleotide sequence that encodes a signal peptide. In some embodiments, an FGFR1 ECD lacks a signal peptide. In some embodiments, an FGFR1 ECD includes at least one signal peptide, which may be a native FGFR1 signal peptide or a heterologous signal peptide.

The term “vector” is used to describe a polynucleotide that may be engineered to contain a cloned polynucleotide or polynucleotides that may be propagated in a host cell. A vector may include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that may be used in colorimetric assays, e.g., β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells; plant cells; and insect cells. Exemplary mammalian cells include, but are not limited to, 293 and CHO cells, and their derivatives, such as 293-6E and DG44 cells, respectively.

The term “isolated” as used herein refers to a molecule that has been separated from at least some of the components with which it is typically found in nature. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated” so long as that polynucleotide is not found in that vector in nature.

The term “anti-neoplastic composition” refers to a composition useful in treating cancer comprising at least one active therapeutic agent, e.g., an “anti-cancer agent.” Examples of therapeutic agents (anti-cancer agents) include, but are not limited to, e.g., chemotherapeutic agents, growth inhibitory agents, cytotoxic agents, agents used in radiation therapy, anti-angiogenic agents, apoptotic agents, anti-tubulin agents, and other agents to treat cancer, such as anti-VEGF antibodies (e.g., bevacizumab, AVASTIN®), anti-HER-2 antibodies (e.g., trastuzumab, HERCEPTIN®), anti-CD20 antibodies (e.g., rituximab, RITUXAN®), an epidermal growth factor receptor (EGFR) antagonist (e.g., a tyrosine kinase inhibitor), HER1/EGFR inhibitors (e.g., erlotinib, TARCEVA®), platelet derived growth factor inhibitors (e.g., GLEEVEC®, imatinib mesylate)), COX-2 inhibitors (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also included in the invention.

A “chemotherapeutic agent” refers to a chemical compound useful in the treatment of cancer. Examples of chemotherapeutic agents include alkylating agents such as thiotepa and cyclophosphamide (e.g., CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (e.g., dronabinol, MARINOL®); beta-lapachone; lapachol; colchicines; betulinic acid; a camptothecin (including the synthetic analogue topotecan (e.g., HYCAMTIN®), CPT-11 (e.g., irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (e.g., DOXIL®), liposomal doxorubicin TLC D-99 (e.g., MYOCET®), pegylated liposomal doxorubicin (e.g., CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (e.g., GEMZAR®), pemetrexed (e.g., ALIMTA®); tegafur (e.g., UFTORAL®), capecitabine (e.g., XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2′-trichlorotriethylamine; trichothecenes (especially T-2 toxin, verracurin A, roridin A and anguidine); urethan; vindesine (e.g., ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (e.g., TAXOL®), albumin-engineered nanoparticle formulation of paclitaxel (e.g., ABRAXANE™), and docetaxel (e.g., TAXOTERE®); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (e.g., VELBAN®), vincristine (e.g., ONCOVIN®), vindesine (e.g., ELDISINE®, FILDESIN®), and vinorelbine (e.g., NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (e.g., TARGRETIN®); bisphosphonates such as clodronate (for example, BONEFOS® or OSTAC®), etidronate (e.g., DIDROCAL®), NE-58095, zoledronic acid/zoledronate (e.g., ZOMETA®), alendronate (e.g., FOSAMAX®), pamidronate (e.g., AREDIA®), tiludronate (e.g., SKELID®), or risedronate (e.g., ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib, e.g., NEXAVAR®; Bayer); SU-11248 (sunitinib, e.g., SUTENT®, Pfizer); perifosine, COX-2 inhibitor (e.g. celecoxib or etoricoxib), proteosome inhibitor (e.g. PS341); bortezomib (e.g., VELCADE®); CCI-779; tipifarnib (R11577); orafenib, ABT510; Bcl-2 inhibitor such as oblimersen sodium (e.g., GENASENSE®); pixantrone; EGFR inhibitors (see definition below); tyrosine kinase inhibitors (see definition below); serine-threonine kinase inhibitors such as rapamycin (e.g., sirolimus, RAPAMUNE®); farnesyltransferase inhibitors such as lonafarnib (e.g., SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (e.g., ELOXATIN®) combined with 5-FU and leucovorin.

Chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens with mixed agonist/antagonist profile, including, tamoxifen (e.g., NOLVADEX®), 4-hydroxytamoxifen, toremifene (e.g., FARESTON®), idoxifene, droloxifene, raloxifene (e.g., EVISTA®), trioxifene, keoxifene, and selective estrogen receptor modulators (SERMs) such as SERM3; pure anti-estrogens without agonist properties, such as fulvestrant (e.g., FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (e.g., AROMASIN®), and nonsteroidal aromatase inhibitors such as anastrazole (e.g., ARIMIDEX®), letrozole (e.g., FEMARA®) and aminoglutethimide, and other aromatase inhibitors include vorozole (e.g., RIVISOR®), megestrol acetate (e.g., MEGASE®), fadrozole, and 4(5)-imidazoles; lutenizing hormone-releasing hormone agonists, including leuprolide (e.g., LUPRON® and ELIGARD®), goserelin, buserelin, and tripterelin; sex steroids, including progestins such as megestrol acetate and medroxyprogesterone acetate, estrogens such as diethylstilbestrol and premarin, and androgens/retinoids such as fluoxymesterone, all transretinoic acid and fenretinide; onapristone; anti-progesterones; estrogen receptor down-regulators (ERDs); anti-androgens such as flutamide, nilutamide and bicalutamide; and pharmaceutically acceptable salts, acids or derivatives of any of the above; as well as combinations of two or more of the above.

An “angiogenic factor or agent” refers to a growth factor which stimulates the development of blood vessels, e.g., promote angiogenesis, endothelial cell growth, stability of blood vessels, and/or vasculogenesis, etc. For example, angiogenic factors, include, but are not limited to, e.g., VEGF and members of the VEGF family (VEGF-B, VEGF-C and VEGF-D), P1GF, PDGF family, fibroblast growth factor family (FGFs), TIE ligands (Angiopoietins), ephrins, delta-like ligand 4 (DLL4), del-1, fibroblast growth factors: acidic (aFGF) and basic (bFGF), follistatin, granulocyte colony-stimulating factor (G-CSF), hepatocyte growth factor (HGF)/scatter factor (SF), interleukin-8 (IL-8), leptin, midkine, neuropilins, placental growth factor, platelet-derived endothelial cell growth factor (PD-ECGF), platelet-derived growth factor, especially PDGF-BB or PDGFR-beta, pleiotrophin (PTN), progranulin, proliferin, transforming growth factor-alpha (TGF-alpha), transforming growth factor-beta (TGF-beta), tumor necrosis factor-alpha (TNF-alpha), etc. It would also include factors that accelerate wound healing, such as growth hormone, insulin-like growth factor-I (IGF-I), VIGF, epidermal growth factor (EGF), CTGF and members of its family, and TGF-alpha and TGF-beta. See, e.g., Klagsbrun and D'Amore (1991) Annu. Rev. Physiol. 53:217-39; Streit and Detmar (2003) Oncogene 22:3172-3179; Ferrara & Alitalo (1999) Nature Medicine 5(12):1359-1364; Tonini et al. (2003) Oncogene 22:6549-6556 (e.g., Table 1 listing known angiogenic factors); and, Sato (2003) Int. J. Clin. Oncol. 8:200-206.

An “anti-angiogenic agent” or “angiogenesis inhibitor” refers to a small molecular weight substance, a polynucleotide (including, e.g., an inhibitory RNA (RNAi or siRNA)), a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenic agent includes those agents that bind and block the angiogenic activity of the angiogenic factor or its receptor. For example, an anti-angiogenic agent is an antibody or other antagonist to an angiogenic agent as defined above, e.g., fusion proteins that binds to VEGF-A such as ZALTRAP™ (Aflibercept), antibodies to VEGF-A such as AVASTIN® (bevacizumab) or to the VEGF-A receptor (e.g., KDR receptor or Flt-1 receptor), VEGF receptor antagonists such as small molecule inhibitors of the VEGFR tyrosine kinases (e.g., pazopanib), anti-PDGFR inhibitors such as GLEEVEC® (imatinib mesylate), small molecules that block VEGF receptor signaling (e.g., PTK787/ZK2284, SU6668, SUTENT®/SU11248 (sunitinib malate), AMG706, or those described in, e.g., international patent application WO 2004/113304). Anti-angiogenic agents also include native angiogenesis inhibitors, e.g., angiostatin, endostatin, etc. See, e.g., Klagsbrun and D'Amore (1991) Annu. Rev. Physiol. 53:217-39; Streit and Detmar (2003) Oncogene 22:3172-3179 (e.g., Table 3 listing anti-angiogenic therapy in malignant melanoma); Ferrara & Alitalo (1999) Nature Medicine 5(12):1359-1364; Tonini et al. (2003) Oncogene 22:6549-6556 (e.g., Table 2 listing known anti-angiogenic factors); and, Sato (2003) Int. J. Clin. dOncol. 8:200-206 (e.g., Table 1 listing anti-angiogenic agents used in clinical trials).

A “VEGF antagonist” refers to a molecule capable of neutralizing, blocking, inhibiting, abrogating, reducing or interfering with VEGF activities including, but not limited to, its binding to one or more VEGF receptors. VEGF antagonists include, without limitation, anti-VEGF antibodies and antigen-binding fragments thereof, receptor molecules and derivatives which bind specifically to VEGF thereby sequestering its binding to one or more receptors, anti-VEGF receptor antibodies, VEGF receptor antagonists such as small molecule inhibitors of the VEGFR tyrosine kinases (e.g., pazopanib) and immunoadhesins that binds to VEGF such as VEGF trap (e.g., aflibercept). The term “VEGF antagonist,” as used herein, specifically includes molecules, including antibodies, antibody fragments, other binding polypeptides, peptides, and non-peptide small molecules, that bind to VEGF and are capable of neutralizing, blocking, inhibiting, abrogating, reducing or interfering with VEGF activities. Thus, the term “VEGF activities” specifically includes VEGF mediated biological activities of VEGF.

The terms “subject” and “patient” are used interchangeably herein to refer to a mammal. In some embodiments, the subject or patient is a human. In other embodiments, methods of treating other mammals, including, but not limited to, rodents, simians, felines, canines, equines, bovines, porcines, ovines, caprines, mammalian laboratory animals, mammalian farm animals, mammalian sport animals, and mammalian pets, are also provided.

The term “sample” or “patient sample” as used herein, refers to a composition that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. By “tissue or cell sample” is meant a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample”, “reference cell”, or “reference tissue”, as used herein, refers to a sample, cell or tissue obtained from a source known, or believed, not to be afflicted with the disease or condition for which a method or composition of the invention is being used to identify. In some embodiments, a reference sample, reference cell or reference tissue is obtained from a healthy part of the body of the same subject or patient in whom a disease or condition is being identified using a composition or method of the invention. In some embodiments, a reference sample, reference cell or reference tissue is obtained from a healthy part of the body of one or more individuals who are not the subject or patient in whom a disease or condition is being identified using a composition or method of the invention.

“Cancer” and “tumor,” as used herein, are interchangeable terms that refer to any abnormal cell or tissue growth or proliferation in an animal. As used herein, the terms “cancer” and “tumor” encompass solid and hematological/lymphatic cancers and also encompass malignant, pre-malignant, and benign growth, such as dysplasia. Examples of cancer include but are not limited to, carcinoma, lymphoma, blastoma, sarcoma, and leukemia. More particular non-limiting examples of such cancers include squamous cell cancer, small-cell lung cancer, pituitary cancer, esophageal cancer, astrocytoma, soft tissue sarcoma, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cancer, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, and various types of head and neck cancer.

A “cell with FGFR1 gene amplification” refers to a cell that comprises more than two copies of the FGFR1 gene. In some embodiments, a cell with FGFR1 gene amplification refers to a cell that has a ratio of FGFR1 gene to chromosome 8 centromere of greater than 1. In some embodiments, the ratio is determined by fluorescence in situ hybridization. “Cancer with FGFR1 gene amplification,” as used herein, refers to a cancer in which at least a portion of the cancer cells have FGFR1 gene amplification. In some embodiments, a cancer with FGFR1 gene amplification refers to a cancer in which at least a portion of the cancer cells comprise at least four copies of the FGFR1 gene. In some embodiments, a cancer with FGFR1 gene amplification refers to a cancer in which at least a portion of the cancer cells have an FGFR1 gene:chromosome 8 centromere ratio of greater than 1. An exemplary FGFR1 gene sequence can be found, e.g., NCBI Reference Sequence: NG_{—}007729.1 dated 23 Mar. 2013.

In some embodiments, a cell with FGFR1 gene amplification comprises at least 3 copies, at least 4 copies, at least 5 copies, at least 6 copies, at least 8 copies, or at least 10 copies of the FGFR1 gene. In some embodiments, a cell with FGFR1 gene amplification comprises at least 4 copies. In some embodiments, a cell with FGFR1 gene amplification has a ratio of FGFR1 gene:chromosome 8 centromere of at least 1.5, at least 2, at least 2.5, at least 3, at least 3.5, or at least 4. In some embodiments, a cell with FGFR1 gene amplification has a ratio of FGFR1 gene:chromosome 8 centromere of at least 2. In some embodiments, a cell with FGFR1 gene amplification has a ratio of FGFR1 gene:chromosome 8 centromere of greater than 2. In some embodiments, each copy of the FGFR1 gene in a cell with FGFR1 gene amplification need not be a complete copy of the FGFR1 gene. In some embodiments, a cell with FGFR1 gene amplification has elevated levels of FGFR1 (i.e., in some embodiments, a cell with FGFR1 gene amplification is also a cell with FGFR1 overexpression).

A “cell with FGFR1 overexpression” or a “cell that overexpresses FGFR1” refers to a cell that has at least a 2-fold greater level of FGFR1 mRNA or protein than a reference cell. A “cancer with FGFR1 overexpression” or a “cancer that overexpresses FGFR1” refers to a cancer in which at least a portion of the cells have at least a 2-fold greater level of FGFR1 mRNA or protein than a reference cell. In some embodiments, a cell with FGFR1 overexpression has at least 3-fold, at least 4-fold, at least 5-fold, at least 7-fold, or at least 10-fold greater level of FGFR1 mRNA or protein than a reference cell. The level of FGFR1 mRNA or protein can be determined by any suitable method including, but not limited to, the methods described herein. In some embodiments, FGFR1 is FGFR1IIIc. An exemplary human FGFR1 protein sequence can be found, e.g., at UniProtKB/Swiss-Prot Reference Sequence: P11362 (FGFR1HUMAN) dated Mar. 21, 2012. An exemplary human FGFR1 mRNA sequence can be found, e.g., at NCBI Reference Sequence: NM_{—}023110.2 dated 24 Mar. 2012. An exemplary human FGFR1IIIc protein sequence can be found, e.g., at NCBI Reference Sequence: NP_{—}075598.2 dated 24 Mar. 2012. An exemplary human FGFR1IIIc mRNA sequence can be found, e.g., at NCBI Reference Sequence: NM_{—}023110.2 dated 24 Mar. 2012.

A “cell with FGFR3 overexpression” or a “cell that overexpresses FGFR3” refers to a cell that has at least a 2-fold greater level of FGFR3 mRNA or protein than a reference cell. A “cancer with FGFR3 overexpression” or a “cancer that overexpresses FGFR3” refers to a cancer in which at least a portion of the cells have at least a 2-fold greater level of FGFR mRNA or protein than a reference cell. In some embodiments, a cell with FGFR3 overexpression has at least 3-fold, at least 4-fold, at least 5-fold, at least 7-fold, or at least 10-fold greater level of FGFR3 mRNA or protein than a reference cell. The level of FGFR3 mRNA or protein can be determined by any suitable method including, but not limited to, the methods described herein. In any of the embodiments described herein, FGFR3 may be FGFR3IIIc. An exemplary human FGFR3IIIc protein sequence can be found, e.g., at NCBI Reference Sequence: NP_{—}000133.1 dated 12 Feb. 2012. An exemplary human FGFR3IIIc mRNA sequence can be found, e.g., at NCBI Reference Sequence: NM_{—}000142.4 dated 12 Feb. 2012.

A “cell with FGFR3 gene amplification” refers to a cell that comprises more than two copies of the FGFR3 gene. In some embodiments, a cell with FGFR3 gene amplification refers to a cell that has a ratio of FGFR3 gene to chromosome 4 centromere of greater than 1. In some embodiments, the ratio is determined by fluorescence in situ hybridization. “Cancer with FGFR3 gene amplification,” as used herein, refers to a cancer in which at least a portion of the cancer cells have FGFR3 gene amplification. In some embodiments, a cancer with FGFR3 gene amplification refers to a cancer in which at least a portion of the cancer cells comprise at least four copies of the FGFR3 gene. In some embodiments, a cancer with FGFR3 gene amplification refers to a cancer in which at least a portion of the cancer cells have an FGFR3 gene:chromosome 4 centromere ratio of greater than 1. An exemplary FGFR3 gene sequence can be found, e.g., NCBI Reference Sequence: NG_{—}012632.1 dated 24 Mar. 2013.

In some embodiments, a cell with FGFR3 gene amplification comprises at least 3 copies, at least 4 copies, at least 5 copies, at least 6 copies, at least 8 copies, or at least 10 copies of the FGFR3 gene. In some embodiments, a cell with FGFR3 gene amplification comprises at least 4 copies. In some embodiments, a cell with FGFR3 gene amplification has a ratio of FGFR3 gene:chromosome 4 centromere of at least 1.5, at least 2, at least 2.5, at least 3, at least 3.5, or at least 4. In some embodiments, a cell with FGFR3 gene amplification has a ratio of FGFR3 gene:chromosome 4 centromere of at least 2. In some embodiments, a cell with FGFR3 gene amplification has a ratio of FGFR3 gene:chromosome 4 centromere of greater than 2. In some embodiments, each copy of the FGFR3 gene in a cell with FGFR3 gene amplification need not be a complete copy of the FGFR3 gene. In some embodiments, a cell with FGFR3 gene amplification has elevated levels of FGFR3 (i.e., in some embodiments, a cell with FGFR3 gene amplification is also a cell with FGFR3 overexpression).

A “cell with FGF2 gene amplification” refers to a cell that comprises more than two copies of the FGF2 gene. In some embodiments, a cell with FGF2 gene amplification refers to a cell that has a ratio of FGF2 gene to chromosome 4 centromere of greater than 1. In some embodiments, the ratio is determined by fluorescence in situ hybridization. “Cancer with FGF2 gene amplification,” as used herein, refers to a cancer in which at least a portion of the cancer cells have FGF2 gene amplification. In some embodiments, a cancer with FGF2 gene amplification refers to a cancer in which at least a portion of the cancer cells comprise at least four copies of the FGF2 gene. In some embodiments, a cancer with FGF2 gene amplification refers to a cancer in which at least a portion of the cancer cells have an FGF2 gene:chromosome 4 centromere ratio of greater than 1. An exemplary FGF2 gene sequence can be found, e.g., NCBI Reference Sequence: NG_{—}029067.1 dated 26 Mar. 2013.

In some embodiments, a cell with FGF2 gene amplification comprises at least 3 copies, at least 4 copies, at least 5 copies, at least 6 copies, at least 8 copies, or at least 10 copies of the FGF2 gene. In some embodiments, a cell with FGF2 gene amplification comprises at least 4 copies. In some embodiments, a cell with FGF2 gene amplification has a ratio of FGF2 gene:chromosome 4 centromere of at least 1.5, at least 2, at least 2.5, at least 3, at least 3.5, or at least 4. In some embodiments, a cell with FGF2 gene amplification has a ratio of FGF2 gene:chromosome 4 centromere of at least 2. In some embodiments, a cell with FGF2 gene amplification has a ratio of FGF2 gene:chromosome 4 centromere of greater than 2. In some embodiments, each copy of the FGF2 gene in a cell with FGF2 gene amplification need not be a complete copy of the FGF2 gene. In some embodiments, a cell with FGF2 gene amplification has elevated levels of FGF2 (i.e., in some embodiments, a cell with FGF2 gene amplification is also a cell with FGF2 overexpression).

A “cell with FGF2 overexpression” or a “cell that overexpresses FGF2” refers to a cell that has at least a 2-fold greater level of FGF2 mRNA or protein than a reference cell. A “cancer with FGF2 overexpression” or a “cancer that overexpresses FGF2” refers to a cancer in which at least a portion of the cells have at least a 2-fold greater level of FGF2 mRNA or protein than a reference cell. In some embodiments, a cell with FGF2 overexpression has at least 3-fold, at least 4-fold, at least 5-fold, at least 7-fold, or at least 10-fold greater level of FGF2 mRNA or protein than a reference cell. The level of FGF2 mRNA or protein can be determined by any suitable method including, but not limited to, the methods described herein. An exemplary human FGF2 protein sequence can be found, e.g., at NCBI Reference Sequence: NP_{—}001997.5 dated 12 Feb. 2012. An exemplary human FGF2 mRNA sequence can be found, e.g., at NCBI Reference Sequence: NM_{—}002006.4 dated 12 Feb. 2012.

A “cell with DKK3 overexpression” or a “cell that overexpresses DKK3” refers to a cell that has at least a 2-fold greater level of DKK3 mRNA or protein than a reference cell. A “cancer with DKK3 overexpression” or a “cancer that overexpresses DKK3” refers to a cancer in which at least a portion of the cells have at least a 2-fold greater level of DKK3 mRNA or protein than a reference cell. In some embodiments, a cell with DKK3 overexpression has at least 3-fold, at least 4-fold, at least 5-fold, at least 7-fold, or at least 10-fold greater level of DKK3 mRNA or protein than a reference cell. The level of DKK3 mRNA or protein can be determined by any suitable method including, but not limited to, the methods described herein. An exemplary human DKK3 protein sequence can be found, e.g., at NCBI Reference Sequence: NP_{—}001018067.1 dated 22 Jan. 2012. An exemplary human DKK3 mRNA sequence can be found, e.g., at NCBI Reference Sequence: NM_{—}001018057.1 dated 22 Jan. 2012.

A “cell with FGF18 overexpression” or a “cell that overexpresses FGF18” refers to a cell that has at least a 2-fold greater level of FGF18 mRNA or protein than a reference cell. A “cancer with FGF18 overexpression” or a “cancer that overexpresses FGF18” refers to a cancer in which at least a portion of the cells have at least a 2-fold greater level of FGF18 mRNA or protein than a reference cell. In some embodiments, a cell with FGF18 overexpression has at least 3-fold, at least 4-fold, at least 5-fold, at least 7-fold, or at least 10-fold greater level of FGF18 mRNA or protein than a reference cell. The level of FGF18 mRNA or protein can be determined by any suitable method including, but not limited to, the methods described herein. An exemplary human FGF18 protein sequence can be found, e.g., at NCBI Reference Sequence: NP_{—}003853 dated 27 Jun. 2012. An exemplary human FGF18 mRNA sequence can be found, e.g., at NCBI Reference Sequence: NM_{—}003862.2 dated 27 Jun. 2012.

A “cell with ETV4 overexpression” or a “cell that overexpresses ETV4” refers to a cell that has at least a 2-fold greater level of ETV4 mRNA or protein than a reference cell. A “cancer with ETV4 overexpression” or a “cancer that overexpresses ETV4” refers to a cancer in which at least a portion of the cells have at least a 2-fold greater level of ETV4 mRNA or protein than a reference cell. In some embodiments, a cell with ETV4 overexpression has at least 3-fold, at least 4-fold, at least 5-fold, at least 7-fold, or at least 10-fold greater level of ETV4 mRNA or protein than a reference cell. The level of ETV4 mRNA or protein can be determined by any suitable method including, but not limited to, the methods described herein. An exemplary human ETV4 protein sequence can be found, e.g., at NCBI Reference Sequence: NP_{—}001977.1 dated 8 Sep. 2012. An exemplary human ETV4 mRNA sequence can be found, e.g., at NCBI Reference Sequence: NM_{—}001986.2 dated 8 Sep. 2012.

A “cancer that is estrogen receptor (ER) positive” or a “cancer that is ER positive” refers to a cancer that has been determined to be ER positive. In some embodiments, a cancer has been determined to be ER positive according to the American Society of Clinical Oncology/College of American Pathologists Guideline Recommendations for Immunohistochemical Testing of Estrogen and Progesterone Receptors in Breast Cancer, J. Clin. Oncol., 2010, 28: 2784-2795. In some embodiments, a cancer is considered to be ER positive when ≧1% of the tumor cell nuclei are immunoreactive in an immunohistochemistry (IHC) assay for the estrogen receptor. In some embodiments, a cancer is considered to be ER positive according to an assay manufacturer's or assay laboratory's guidelines.

A “cancer that is progesterone receptor (PR) positive” or a “cancer that is PR positive” refers to a cancer that has been determined to be PR positive. In some embodiments, a cancer has been determined to be PR positive according to the American Society of Clinical Oncology/College of American Pathologists Guideline Recommendations for Immunohistochemical Testing of Estrogen and Progesterone Receptors in Breast Cancer, J. Clin. Oncol., 2010, 28: 2784-2795. In some embodiments, a cancer is considered to be PR positive when ≧1% of the tumor cell nuclei are immunoreactive in an immunohistochemistry (IHC) assay for the progesterone receptor. In some embodiments, a cancer is considered to be PR positive according to an assay manufacturer's or assay laboratory's guidelines.

A “cancer that is HER2 positive” refers to a cancer that has been determined to be HER2 positive. In some embodiments, a cancer that has been determined to be HER2 positive using an immunohistochemistry (IHC) assay for the HER2 protein, and/or a fluorescent in situ hybridization (FISH) assay to detect HER2 gene amplification. In some embodiments, a cancer is characterized as HER2 positive when the IHC cell membrane stain intensity is 3+ on a scale from 0 to 3+. In some embodiments, a HER2 FISH assay is used to determine whether the HER2 gene is amplified. In some such embodiments, the HER2 gene is considered to be amplified when the ratio of copies of the HER2 gene to chromosome 17 centromere is greater than 2. In some embodiments, if the HER2 gene is amplified, the breast cancer is considered to be HER2 positive, regardless of the results of an IHC assay. In some embodiments, a cancer is considered to be HER2 positive according to an assay manufacturer's or assay laboratory's guidelines.

A “cell with HER2 gene amplification” refers to a cell that comprises more than two copies of the HER2 gene. In some embodiments, a cell with HER2 gene amplification refers to a cell that has a ratio of HER2 gene to chromosome 17 centromere of greater than 1. In some embodiments, the ratio is determined by fluorescence in situ hybridization. “Cancer with HER2 gene amplification,” as used herein, refers to a cancer in which at least a portion of the cancer cells have HER2 gene amplification. In some embodiments, a cancer with HER2 gene amplification refers to a cancer in which at least a portion of the cancer cells comprise at least four copies of the HER2 gene. In some embodiments, a cancer with HER2 gene amplification refers to a cancer in which at least a portion of the cancer cells have an HER2 gene:chromosome 17 centromere ratio of greater than 1. An exemplary HER2 gene sequence can be found, e.g., NCBI Reference Sequence: NG_{—}007503.1 dated 22 Apr. 2013. In some embodiments, HER2 gene amplification is determined according to Persons, et al. “Fluorescence in situ hybridization (FISH) for detection of HER-2/neu amplification in breast cancer: a multicenter portability study.” Ann Clin Lab Sci 30: 41-48 (2000), which is incorporated by reference herein in its entirety for any purpose.

In some embodiments, a cell with HER2 gene amplification comprises at least 3 copies, at least 4 copies, at least 5 copies, at least 6 copies, at least 8 copies, or at least 10 copies of the HER2 gene. In some embodiments, a cell with HER2 gene amplification comprises at least 4 copies. In some embodiments, a cell with HER2 gene amplification has a ratio of HER2 gene:chromosome 17 centromere of at least 1.5, at least 2, at least 2.5, at least 3, at least 3.5, or at least 4. In some embodiments, a cell with HER2 gene amplification has a ratio of HER2 gene:chromosome 17 centromere of at least 2. In some embodiments, a cell with HER2 gene amplification has a ratio of HER2 gene:chromosome 17 centromere of greater than 2. In some embodiments, each copy of the HER2 gene in a cell with HER2 gene amplification need not be a complete copy of the HER2 gene. In some embodiments, a cell with HER2 gene amplification has elevated levels of HER2 (i.e., in some embodiments, a cell with HER2 gene amplification is also a cell with HER2 overexpression). In some embodiments, a cancer in which at least a portion of the cells have HER2 gene amplification and/or HER2 overexpression is considered to be HER2 positive.

A “cancer that is p95HER2 positive” refers to a cancer in which at least a portion of the cancer cells contain p95HER2, as determined by immunohistochemistry (IHC), Western blot, or VeraTag® assay (Monogram Biosciences). See, e.g., Han et al., PLoS One, 2012, 7(7): e39943; Parra-Palau et al., Cancer Res., 2010, 70: 8537-8546; Saez et al., Clin. Cancer Res., 2006, 12(2): 424-431; Sperinde et al., Clin. Canc. Res., 2010, 16(16): 4226-4235; and U.S. Pat. No. 8,389,227 B2. In some embodiments, a cancer is determined to be p95HER2 positive by IHC. In some such embodiments, a cancer is determined to be p95HER2 positive using the methods described in Sperinde et al., Clin. Canc. Res., 2010, 16(16): 4226-4235, such as methods using anti-p95 antibody clone D9 in a VeraTag assay. In some embodiments, a cancer is determined to be p95HER2 positive using the methods described in U.S. Pat. No. 8,389,227 B2, such as methods using an antibody produced by a hybridoma cell line deposited with the Deutschland Sammlung von Mikroorganismen and Zellen under accession number DSM ACC2904 or DSM ACC2980. In some embodiments, a cancer is determined to be p95HER2 positive according to the assay manufacturer's or assay laboratory's guidelines. p95HER2 refers to a collection of carboxy-terminal HER2 fragments, which, in some embodiments, may be divided into 95- to 100-kDa fragments and 100- to 115-kDa fragments. See, e.g., Arribas et al., Cancer Res., 2011, 71: 1515-1519. In some embodiments, a cancer that is p95HER2 positive contains 100- to 115-kDa fragments of HER2.

An “aromatase inhibitor” refers to a molecule capable of neutralizing, blocking, inhibiting, abrogating, reducing or interfering with aromatase activities including, but not limited to, its ability to convert androgens (such as testosterone and androstenedione) into estrogens (such as estradiol and estrone). Nonlimiting exemplary aromatase inhibitors include aminoglutethimide, testolactone (e.g., Teslac®), anastrozole (e.g., Arimidex®), letrozole (e.g., Femara®), exemestane (e.g., Aromasin®), vorozole (e.g., Rivisor®), formestane (e.g., Lentaron®), megestrol acetate (e.g., Megase®), fadrozole (e.g., Afema®), 4-hydroxyandrostenedione (4-OHA), 1,4,6-androstatrien-3,17-dione (ATD), and 4-androstene-3,6,17-trione (6-OXO).

“Gonadotropin-releasing hormone agonist” and “GnRH agonist” refer to a molecule capable of stimulating or enhancing gonadotropin-releasing hormone receptor activities, including, but not limited to, eliciting release of luteinizing hormone (LH) and/or follicle-stimulating hormone (FSH) from the pituitary. Nonlimiting exemplary gonadotropin-releasing hormone agonists include leuprolide (e.g., Lupron®, Eligard®), buserelin (e.g., Suprefact®, Suprecor®), histrelin (e.g., Supprelin LA®, Vantas®), goserelin acetate (e.g., Zoladex®), deslorelin (e.g., Suprelorin®, Ovuplant®), nafarelin (e.g., Synarel®), and triptorelin.

“Gonadotropin-releasing hormone antagonist” and “GnRH antagonist” refer to a molecule capable of neutralizing, blocking, inhibiting, abrogating, reducing or interfering with gonadotropin-releasing hormone activities including, but not limited to, eliciting release of luteinizing hormone (LH) and/or follicle-stimulating hormone (FSH). Nonlimiting exemplary gonadotropin-releasing hormone antagonists include cetrorelix (e.g., Cetrotide®), ganirelix (e.g., Antagon®), abarelix (e.g., Plenaxis®), and degarelix (e.g., Firmagon®).

“Androgen receptor inhibitor” and “AR inhibitor” refer to a molecule capable of neutralizing, blocking, inhibiting, abrogating, reducing or interfering with androgen receptor activities including, but not limited to, its ability to act as a transcription factor (i.e., regulate gene expression). Nonlimiting exemplary androgen receptor inhibitors include cyproterone acetate (e.g., Androcur®, Cyprostat®), flutamide (e.g., Eulexin®), bicalutamide (e.g., Casodex®), enzalutamide (e.g., Xtandi®), ketoconazole, and nilutamide (e.g., Anandron®, Nilandron®).

“17-hydroxylase inhibitor” refers to a molecule capable of neutralizing, blocking, inhibiting, abrogating, reducing or interfering with cytochrome P450 17A1 (also referred to as steroid 17-alpha-monooxygenase) activities including, but not limited to, its ability to add a hydroxyl group to carbon 17 of the steroid D ring of pregnenolone or progesterone. A nonlimiting exemplary 17-hydroxylase inhibitor is abiraterone acetate (e.g., Zytiga®).

“Estrogen receptor antagonist” and “ER antagonist” refer to a molecule capable of neutralizing, blocking, inhibiting, abrogating, reducing or interfering with estrogen receptor activities including, but not limited to, its ability to act as a transcription factor (i.e., regulate gene expression). Nonlimiting exemplary ER antagonists include tamoxifen (e.g., Nolvadex®, Istubal®, and Valodex®) and fulvestrant (e.g., Faslodex®).

“Treatment,” as used herein, includes any administration or application of a therapeutic for condition in a mammal, including a human, and includes inhibiting the condition or progression of the condition, inhibiting or slowing the condition or its progression, arresting its development, partially or fully relieving the condition, or curing the condition, for example, by causing regression, or restoring or repairing a lost, missing, or defective function; or stimulating an inefficient process. In some embodiments, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

An “effective amount” or “therapeutically effective amount” of a molecule or a combination of molecules means an amount that is sufficient to treat a condition and/or to inhibit growth of tumor cells in at least a subset of subjects when given alone or in combination with other treatments. In certain embodiments, a therapeutically effective amount refers to an amount effective, at dosages and for periods of time necessary, to achieve the desired therapeutic or prophylactic result. A therapeutically effective amount of FGFR1 fusion protein of the invention may vary according to factors such as the disease state, age, sex, and weight of the individual, and the ability of FGFR1 fusion protein to elicit a desired response in the individual. A therapeutically effective amount is also one in which any toxic or detrimental effects of the FGFR1 fusion proteins are outweighed by the therapeutically beneficial effects. In the case of cancer, the effective amount of the drug may reduce the number of cancer cells; reduce the tumor size; inhibit (i.e., slow to some extent and typically stop) cancer cell infiltration into peripheral organs; inhibit (i.e., slow to some extent and typically stop) tumor metastasis; inhibit, to some extent, tumor growth; allow for treatment of the tumor, and/or relieve to some extent one or more of the symptoms associated with the disorder. To the extent the drug may prevent growth and/or kill existing cancer cells, it may be cytostatic and/or cytotoxic.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The terms “inhibition” or “inhibit” refer to a decrease or cessation of any phenotypic characteristic or to the decrease or cessation in the incidence, degree, or likelihood of that characteristic. Nonlimiting exemplary inhibition includes inhibition of tumor growth.

The terms “benefit”, “clinical benefit”, “responsiveness”, and “therapeutic responsiveness” as used herein in the context of benefiting from or responding to administration of a therapeutic agent, can be measured by assessing various endpoints, e.g., inhibition, to some extent, of disease progression, including slowing down and complete arrest; reduction in the number of disease episodes and/or symptoms; reduction in lesion size; inhibition (i.e., reduction, slowing down or complete stopping) of disease cell infiltration into adjacent peripheral organs and/or tissues; inhibition (i.e. reduction, slowing down or complete stopping) of disease spread; decrease of auto-immune response, which may, but does not have to, result in the regression or ablation of the disease lesion; relief, to some extent, of one or more symptoms associated with the disorder; increase in the length of disease-free presentation following treatment, e.g., progression-free survival; increased overall survival; higher response rate; and/or decreased mortality at a given point of time following treatment.

Administration “in combination with” one or more further therapeutic agents includes concurrent (including simultaneous) and consecutive (i.e., sequential) administration in any order.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause injection site reaction.

### Therapeutic Compositions and Methods

Methods of Treating Cancer Using FGFR1 ECDs and/or FGFR1 ECD Fusion Molecules

In some embodiments, the invention provides methods of treating cancers in which at least a portion of the cancer cells have FGFR1 gene amplification, FGFR1 overexpression, FGFR3 gene amplification, FGFR3 overexpression, FGF2 overexpression, and/or FGF2 gene amplification. Such cancers have been found, in some embodiments, to be particularly responsive to treatment with a fibroblast growth factor receptor 1 (FGFR1) extracellular domain (ECD) or FGFR1 ECD fusion molecule. Accordingly, in some embodiments, a method of treating cancer having FGFR1 gene amplification, FGFR1 overexpression, FGFR3 gene amplification, FGFR3 overexpression, FGF2 overexpression, and/or FGF2 gene amplification comprises administering a therapeutically effective amount of an FGFR1 ECD or an FGFR1 ECD fusion molecule to the subject. In some embodiments, a method of treating cancer in a subject comprises administering a therapeutically effective amount of a fibroblast growth factor receptor 1 (FGFR1) extracellular domain (ECD) or an FGFR1 ECD fusion molecule to the subject, wherein, prior to administration of the FGFR1 ECD or FGFR1 ECD fusion molecule, at least a portion of the cells of the cancer have been determined to have FGFR1 gene amplification, FGFR1 overexpression, FGFR3 gene amplification, FGFR3 overexpression, FGF2 overexpression, and/or FGF2 gene amplification. In such methods, FGFR1 gene amplification, FGFR1 overexpression, FGFR3 gene amplification, FGFR3 overexpression, FGF2 overexpression, and/or FGF2 gene amplification in a cancer is indicative of therapeutic responsiveness by the cancer to an FGFR1 ECD or FGFR1 ECD fusion molecule.

In some embodiments, the invention provides methods of treating cancers in which at least a portion of the cancer cells have overexpression of at least one, at least two, at least three, or at least four markers selected from FGFR1, FGFR3, FGF2, DKK3, FGF18, and ETV4. In some embodiments, FGFR1 is FGFR1IIIc. In some embodiments, FGFR3 is FGFR3IIIc. In some embodiments, the overexpression is mRNA overexpression. In some embodiments, the overexpression is protein overexpression. In some embodiments, a method of treating cancer that overexpresses at least one marker selected from FGFR1, FGFR3, FGF2, DKK3, FGF18, and ETV4 comprises administering a therapeutically effective amount of an FGFR1 ECD or an FGFR1 ECD fusion molecule to the subject. In some embodiments, a method of treating cancer in a subject comprises administering a therapeutically effective amount of a fibroblast growth factor receptor 1 (FGFR1) extracellular domain (ECD) or an FGFR1 ECD fusion molecule to the subject, wherein, prior to administration of the FGFR1 ECD or FGFR1 ECD fusion molecule, at least a portion of the cells of the cancer have been determined to have overexpression of at least one marker selected from FGFR1, FGFR3, FGF2, DKK3, FGF18, and ETV4. In such methods, FGFR1, FGFR3, FGF2, DKK3, FGF18, and/or ETV4 overexpression in a cancer is indicative of therapeutic responsiveness by the cancer to an FGFR1 ECD or FGFR1 ECD fusion molecule. In some embodiments, FGFR1 is FGFR1IIIc. In some embodiments, FGFR3 is FGFR3IIIc.

In some embodiments, in a cancer with an FGFR1 gene amplification, at least a portion of the cancer cells comprise at least four copies of the FGFR1 gene. In some embodiments, in a cancer with an FGFR1 gene amplification, at least a portion of the cancer cells comprise at least five, at least six, at least 8, or at least 10 copies of the FGFR1 gene. Determination of the FGFR1 gene copy number can be carried out by any suitable method in the art. Certain nonlimiting exemplary methods are discussed herein. In some embodiments, in a cancer with an FGFR1 gene amplification, at least a portion of the cancer cells have a ratio of FGFR1 gene to chromosome 8 centromere of at least 2. In some embodiments, in a cancer with an FGFR1 gene amplification, at least a portion of the cancer cells have a ratio of FGFR1 gene to chromosome 8 centromere of greater than 2. In some embodiments, in a cancer with an FGFR1 gene amplification, at least a portion of the cancer cells have a ratio of FGFR1 gene to chromosome 8 centromere of at least 2.5, at least 3, at least 3.5, or at least 4. Determination of such a ratio can be carried out by any suitable method in the art. Certain nonlimiting exemplary methods are discussed herein.

In some embodiments, in a cancer with an FGF2 gene amplification, at least a portion of the cancer cells comprise at least four copies of the FGF2 gene. In some embodiments, in a cancer with an FGF2 gene amplification, at least a portion of the cancer cells comprise at least five, at least six, at least 8, or at least 10 copies of the FGF2 gene. Determination of the FGF2 gene copy number can be carried out by any suitable method in the art. Certain nonlimiting exemplary methods are discussed herein. In some embodiments, in a cancer with an FGF2 gene amplification, at least a portion of the cancer cells have a ratio of FGF2 gene to chromosome 4 centromere of at least 2. In some embodiments, in a cancer with an FGF2 gene amplification, at least a portion of the cancer cells have a ratio of FGF2 gene to chromosome 4 centromere of greater than 2. In some embodiments, in a cancer with an FGF2 gene amplification, at least a portion of the cancer cells have a ratio of FGF2 gene to chromosome 4 centromere of at least 2.5, at least 3, at least 3.5, or at least 4. Determination of such a ratio can be carried out by any suitable method in the art. Certain nonlimiting exemplary methods are discussed herein.

In some embodiments, in a cancer with an FGFR3 gene amplification, at least a portion of the cancer cells comprise at least four copies of the FGFR3 gene. In some embodiments, in a cancer with an FGFR3 gene amplification, at least a portion of the cancer cells comprise at least five, at least six, at least 8, or at least 10 copies of the FGFR3 gene. Determination of the FGFR3 gene copy number can be carried out by any suitable method in the art. Certain nonlimiting exemplary methods are discussed herein. In some embodiments, in a cancer with an FGFR3 gene amplification, at least a portion of the cancer cells have a ratio of FGFR3 gene to chromosome 4 centromere of at least 2. In some embodiments, in a cancer with an FGFR3 gene amplification, at least a portion of the cancer cells have a ratio of FGFR3 gene to chromosome 4 centromere of greater than 2. In some embodiments, in a cancer with an FGFR3 gene amplification, at least a portion of the cancer cells have a ratio of FGFR3 gene to chromosome 4 centromere of at least 2.5, at least 3, at least 3.5, or at least 4. Determination of such a ratio can be carried out by any suitable method in the art. Certain nonlimiting exemplary methods are discussed herein.

In some embodiments, the cancer is selected from prostate cancer, breast cancer, ovarian cancer, carcinoid cancer, colorectal cancer, lung cancer, brain cancer, endometrial cancer, head and neck cancer, laryngeal cancer, liver cancer, renal cancer, glioblastoma, and pancreatic cancer. In certain embodiments, the cancer is selected from prostate cancer, breast cancer, ovarian cancer, and carcinoid cancer.

In some embodiments, methods of treating breast cancer in a subject are provided, comprising administering a therapeutically effective amount of an FGFR1 ECD or an FGFR1 ECD fusion molecule to a subject with breast cancer. In some embodiments, the breast cancer has been determined to have FGFR1 gene amplification, FGFR1 overexpression, FGFR3 gene amplification, FGFR3 overexpression, FGF2 overexpression, and/or FGF2 gene amplification. In some embodiments, the breast cancer has further been determined to be estrogen receptor (ER) positive and/or progesterone (PR) positive. In some embodiments, the breast cancer has been determined to be HER2 positive. In some embodiments, the breast cancer has been determined to be p95HER2 positive. In some embodiments, the breast cancer is HER2 negative. In any of the embodiments described herein, the breast cancer may be metastatic breast cancer. In some embodiments, a breast cancer that is ER positive and HER2 negative is a metastatic breast cancer. In any of the embodiments described herein, the subject with breast cancer is post-menopausal.

In some embodiments, the subject with breast cancer has previously been administered, or is currently being administered, trastuzumab and/or lapatinib. That is, in some embodiments, the subject with breast cancer previously underwent therapy with trastuzumab and/or lapatinib, but completed or terminated that therapy prior to being administered a therapeutically effective amount of an FGFR1 ECD or an FGFR1 ECD fusion molecule. In some embodiments, the subject with breast cancer receives trastuzumab and/or lapatinib therapy concurrently with FGFR1 ECD or FGFR1 ECD fusion molecule therapy. By “concurrently” it is meant that there is a time period during which both (or all) agents exert their biological activities.

In some embodiments, the subject with breast cancer has previously been administered, or is currently being administered, an aromatase inhibitor. That is, in some embodiments, the subject with breast cancer previously underwent therapy with an aromatase inhibitor, but completed or terminated that therapy prior to being administered a therapeutically effective amount of an FGFR1 ECD or an FGFR1 ECD fusion molecule. In some embodiments, the subject with breast cancer receives aromatase inhibitor therapy concurrently with FGFR1 ECD or FGFR1 ECD fusion molecule therapy. By “concurrently” it is meant that there is a time period during which both (or all) agents exert their biological activities. Nonlimiting exemplary aromatase inhibitors include aminoglutethimide, testolactone (e.g., Teslac®), anastrozole (e.g., Arimidex®), letrozole (e.g., Femara®), exemestane (e.g., Aromasin®), vorozole (e.g., Rivisor®), formestane (e.g., Lentaron®), megestrol acetate (e.g., Megase®), fadrozole (e.g., Afema®), 4-hydroxyandrostenedione (4-OHA), 1,4,6-androstatrien-3,17-dione (ATD), and 4-androstene-3,6,17-trione (6-OXO). In some embodiments, a subject with breast cancer has previously been administered, or is currently being administered, an aromatase inhibitor selected from aminoglutethimide, testolactone (e.g., Teslac®), anastrozole (e.g., Arimidex®), letrozole (e.g., Femara®), exemestane (e.g., Aromasin®), vorozole (e.g., Rivisor®), formestane (e.g., Lentaron®), megestrol acetate (e.g., Megase®), and fadrozole (e.g., Afema®).

In some embodiments, the subject with breast cancer has previously been administered, or is currently being administered, an ER antagonist. In some embodiments, the subject has been determined to have ER positive breast cancer. That is, in some embodiments, the subject with breast cancer previously underwent therapy with an ER antagonist, but completed or terminated that therapy prior to being administered a therapeutically effective amount of an FGFR1 ECD or an FGFR1 ECD fusion molecule. In some embodiments, the subject with breast cancer receives an ER antagonist therapy concurrently with FGFR1 ECD or FGFR1 ECD fusion molecule therapy. By “concurrently” it is meant that there is a time period during which both (or all) agents exert their biological activities. Nonlimiting exemplary ER antagonists include tamoxifen (e.g., Nolvadex®, Istabul®, Valodex®) and fulvestrant (e.g., Faslodex®).

In some embodiments, methods of treating prostate cancer in a subject are provided, comprising administering a therapeutically effective amount of an FGFR1 ECD or an FGFR1 ECD fusion molecule to a subject with prostate cancer. In some embodiments, the prostate cancer has been determined to have FGFR1 gene amplification, FGFR1 overexpression, FGFR3 gene amplification, FGFR3 overexpression, FGF2 overexpression, and/or FGF2 gene amplification. In some such embodiments, the subject with prostate cancer has previously been administered, or is currently being administered, a therapeutic agent selected from a gonadotropin releasing hormone (GnRH) agonist, a GnRH antagonist, an androgen receptor (AR) inhibitor, a 17-hydroxylast inhibitor, and diethylstilbestrol (DES). In some such embodiments, the subject with prostate cancer has previously been administered, or is currently being administered, a therapeutic agent selected from a gonadotropin releasing hormone (GnRH) agonist or a GnRH antagonist.

In some embodiments, the subject with prostate cancer has previously been administered, or is currently being administered, a GnRH agonist. That is, in some embodiments, the subject with prostate cancer previously underwent therapy with a GnRH agonist, but completed or terminated that therapy prior to being administered a therapeutically effective amount of an FGFR1 ECD or an FGFR1 ECD fusion molecule. In some embodiments, the subject with prostate cancer receives GnRH agonist therapy concurrently with FGFR1 ECD or FGFR1 ECD fusion molecule therapy. By “concurrently” it is meant that there is a time period during which both (or all) agents exert their biological activities. Nonlimiting exemplary GnRH agonists include leuprolide (e.g., Lupron®, Eligard®), buserelin (e.g., Suprefact®, Suprecor®), histrelin (e.g., Supprelin LA®, Vantas®), goserelin acetate (e.g., Zoladex®), deslorelin (e.g., Suprelorin®, Ovuplant®), nafarelin (e.g., Synarel®), and triptorelin.

In some embodiments, the subject with prostate cancer has previously been administered, or is currently being administered, a GnRH antagonist. That is, in some embodiments, the subject with prostate cancer previously underwent therapy with a GnRH antagonist, but completed or terminated that therapy prior to being administered a therapeutically effective amount of an FGFR1 ECD or an FGFR1 ECD fusion molecule. In some embodiments, the subject with prostate cancer receives GnRH antagonist therapy concurrently with FGFR1 ECD or FGFR1 ECD fusion molecule therapy. By “concurrently” it is meant that there is a time period during which both (or all) agents exert their biological activities. Nonlimiting exemplary GnRH antagonists include cetrorelix (e.g., Cetrotide®), ganirelix (e.g., Antagon®), abarelix (e.g., Plenaxis®), and degarelix (e.g., Firmagon®).

In some embodiments, the subject with prostate cancer has previously been administered, or is currently being administered, an AR inhibitor. That is, in some embodiments, the subject with prostate cancer previously underwent therapy with an AR inhibitor, but completed or terminated that therapy prior to being administered a therapeutically effective amount of an FGFR1 ECD or an FGFR1 ECD fusion molecule. In some embodiments, the subject with prostate cancer receives AR inhibitor therapy concurrently with FGFR1 ECD or FGFR1 ECD fusion molecule therapy. By “concurrently” it is meant that there is a time period during which both (or all) agents exert their biological activities. Nonlimiting exemplary AR inhibitors include cyproterone acetate (e.g., Androcur®, Cyprostat®), flutamide (e.g., Eulexin®), bicalutamide (e.g., Casodex®), enzalutamide (e.g., Xtandi®), ketoconazole, and nilutamide (e.g., Anandron®, Nilandron®).

In some embodiments, the subject with prostate cancer has previously been administered, or is currently being administered, a 17-hydroxylase inhibitor. That is, in some embodiments, the subject with prostate cancer previously underwent therapy with a 17-hydroxylase inhibitor, but completed or terminated that therapy prior to being administered a therapeutically effective amount of an FGFR1 ECD or an FGFR1 ECD fusion molecule. In some embodiments, the subject with prostate cancer receives 17-hydroxylase inhibitor therapy concurrently with FGFR1 ECD or FGFR1 ECD fusion molecule therapy. By “concurrently” it is meant that there is a time period during which both (or all) agents exert their biological activities. A nonlimiting exemplary 17-hydroxylase inhibitor is abiraterone acetate (e.g., Zytiga®).

In some embodiments, the subject with prostate cancer has previously been administered, or is currently being administered, diethylstilbestrol (DES). That is, in some embodiments, the subject with prostate cancer previously underwent therapy with DES, but completed or terminated that therapy prior to being administered a therapeutically effective amount of an FGFR1 ECD or an FGFR1 ECD fusion molecule. In some embodiments, the subject with prostate cancer receives DES therapy concurrently with FGFR1 ECD or FGFR1 ECD fusion molecule therapy. By “concurrently” it is meant that there is a time period during which both (or all) agents exert their biological activities.

In some embodiments, methods of treating carcinoid cancer in a subject are provided, comprising administering a therapeutically effective amount of an FGFR1 ECD or an FGFR1 ECD fusion molecule to a subject with carcinoid cancer. In some embodiments, the carcinoid cancer has been determined to have FGFR1 gene amplification, FGFR1 overexpression, FGFR3 gene amplification, FGFR3 overexpression, FGF2 overexpression, and/or FGF2 gene amplification. In some embodiments, the subject with carcinoid cancer has previously been administered, or is currently being administered, octreotide (Sandostatin®). That is, in some embodiments, the subject with carcinoid cancer previously underwent therapy with a therapeutically effective amount of octreotide, but completed or terminated that therapy prior to being administered a therapeutically effective amount of an FGFR1 ECD or an FGFR1 ECD fusion molecule. In some embodiments, the subject with prostate cancer receives octreotide therapy concurrently with FGFR1 ECD or FGFR1 ECD fusion molecule therapy. By “concurrently” it is meant that there is a time period during which both (or all) agents exert their biological activities.

In some embodiments, methods of treating ovarian cancer in a subject are provided, comprising administering a therapeutically effective amount of an FGFR1 ECD or an FGFR1 ECD fusion molecule to a subject with ovarian cancer. In some embodiments, the ovarian cancer has been determined to have FGFR1 gene amplification, FGFR1 overexpression, FGFR3 gene amplification, FGFR3 overexpression, FGF2 overexpression, and/or FGF2 gene amplification. In some embodiments, the subject with ovarian cancer has previously been administered, or is currently being administered, an ER antagonist or an aromatase inhibitor. In some embodiments, the ovarian cancer has further been determined to be estrogen receptor (ER) positive and/or progesterone (PR) positive.

In some embodiments, the subject with ovarian cancer has previously been administered, or is currently being administered, an ER antagonist. That is, in some embodiments, the subject with ovarian cancer previously underwent therapy with an ER antagonist, but completed or terminated that therapy prior to being administered a therapeutically effective amount of an FGFR1 ECD or an FGFR1 ECD fusion molecule. In some embodiments, the subject with ovarian cancer receives an ER antagonist therapy concurrently with FGFR1 ECD or FGFR1 ECD fusion molecule therapy. By “concurrently” it is meant that there is a time period during which both (or all) agents exert their biological activities. Nonlimiting exemplary ER antagonists include tamoxifen (e.g., Nolvadex®, Istabul®, Valodex®) and fulvestrant (e.g., Faslodex®).

In some embodiments, the subject with ovarian cancer has previously been administered, or is currently being administered, an aromatase inhibitor. That is, in some embodiments, the subject with ovarian cancer previously underwent therapy with an aromatase inhibitor, but completed or terminated that therapy prior to being administered a therapeutically effective amount of an FGFR1 ECD or an FGFR1 ECD fusion molecule. In some embodiments, the subject with ovarian cancer receives aromatase inhibitor therapy concurrently with FGFR1 ECD or FGFR1 ECD fusion molecule therapy. By “concurrently” it is meant that there is a time period during which both (or all) agents exert their biological activities. Nonlimiting exemplary aromatase inhibitors include aminoglutethimide, testolactone (e.g., Teslac®), anastrozole (e.g., Arimidex®), letrozole (e.g., Femara®), exemestane (e.g., Aromasin®), vorozole (e.g., Rivisor®), formestane (e.g., Lentaron®), megestrol acetate (e.g., Megase®), fadrozole (e.g., Afema®), 4-hydroxyandrostenedione (4-OHA), 1,4,6-androstatrien-3,17-dione (ATD), and 4-androstene-3,6,17-trione (6-OXO). In some embodiments, a subject with ovarian cancer has previously been administered, or is currently being administered, an aromatase inhibitor selected from aminoglutethimide, testolactone (e.g., Teslac®), anastrozole (e.g., Arimidex®), letrozole (e.g., Femara®), exemestane (e.g., Aromasin®), vorozole (e.g., Rivisor®), formestane (e.g., Lentaron®), megestrol acetate (e.g., Megase®), and fadrozole (e.g., Afema®).

In any of the embodiments described herein, at least a portion of the cells of the cancer may have an FGFR1 gene amplification and/or an FGFR3 gene amplification and/or an FGF2 gene amplification. In some embodiments, at least a portion of the cells may comprise at least three, at least four, at least five, at least six, or at least eight copies of the respective gene. In some embodiments, at least a portion of the cells of the cancer may have a ratio of the respective gene to its chromosome centromere of at least 2, at least 2.5, at least 3, at least 3.5, or at least 4. In some embodiments, at least a portion of the cells of the cancer may have a ratio of the respective gene to its chromosome centromere of greater than 2. Gene amplification may be determined by any method in the art, including but not limited to, methods that comprise fluorescent in situ hybridization (FISH). Certain nonlimiting exemplary methods of determining gene amplification are described herein.

In any of the embodiments described herein, at least a portion of the cells of the cancer may have FGFR1 overexpression and/or FGFR3 overexpression and/or FGF2 overexpression. In some embodiments, FGFR1 is FGFR1IIIc. In some embodiments, FGFR3 is FGFR3IIIc. In any of the embodiments described herein, at least a portion of the cells of the cancer may have DKK3 overexpression and/or FGF18 overexpression and/or ETV4 overexpression. Such overexpression may be mRNA overexpression and/or protein overexpression. mRNA overexpression may be determined by any method in the art, including but not limited to, methods comprising quantitative RT-PCR. Protein overexpression may be determined by any method in the art, including but not limited to, methods comprising immunohistochemistry. Certain nonlimiting exemplary methods of determining mRNA and/or protein overexpression are described herein.

In some embodiments, the FGFR1 ECD has an amino acid sequence selected from SEQ ID NOs: 1 to 4. In some embodiments, the FGFR1 ECD has an amino acid sequence selected from SEQ ID NOs: 2 and 4. In some embodiments, the FGFR1 ECD fusion molecule has an amino acid sequence selected from SEQ ID NOs: 5 and 6. In some embodiments, the FGFR1 ECD fusion molecule is FGFR1 ECD.339-Fc with an amino acid sequence of SEQ ID NO: 6.

In some embodiments, an FGFR1 ECD or FGFR1 ECD fusion molecule is administered with one or more additional anti-cancer therapies. Examples of the additional anti-cancer therapies include, without limitation, surgery, radiation therapy (radiotherapy), biotherapy, immunotherapy, and chemotherapy or a combination of these therapies. In addition, cytotoxic agents, anti-angiogenic and anti-proliferative agents can be used in combination with the FGFR1 ECD or FGFR1 ECD fusion molecule. In certain aspects of any of the methods and uses, the invention provides treating cancer in which at least a portion of the cancer cells comprise FGFR1 gene amplification, FGFR1 overexpression, FGFR3 gene amplification, FGFR3 overexpression, FGF2 gene amplification and/or FGF2 overexpression and/or overexpress at least one, at least two, or three markers selected from DKK3, FGF18, and ETV4, by administering therapeutically effective amounts of an FGFR1 ECD and/or FGFR1 ECD fusion molecule and one or more chemotherapeutic agents to a subject. In some embodiments, the subject's cancer has not previously been treated. In some embodiments, the subject's cancer has previously been treated, or is currently being treated, with one or more chemotherapeutic agents. A variety of chemotherapeutic agents may be used in the combined treatment methods and uses of the invention. An exemplary and non-limiting list of chemotherapeutic agents contemplated is provided herein under “Definitions” and in the “Summary of the Invention.” In some embodiments, the invention provides methods of treating cancer, by administering therapeutically effective amounts of an FGFR1 ECD and/or FGFR1 ECD fusion molecule and one or more anti-angiogenic agent(s) to a subject. In some embodiments, the invention provides treating cancer, by administering therapeutically effective amounts of an FGFR1 ECD and/or FGFR1 ECD fusion molecule and one or more VEGF antagonists to a subject. In some embodiments, the invention provides treating cancer, by administering therapeutically effective amounts of an FGFR1 ECD and/or FGFR1 ECD fusion molecule and one or more VEGF antagonists in combination with one or more chemotherapeutic agents to a subject. In some embodiments, the one or more VEGF antagonists are small molecule inhibitors of the VEGFR tyrosine kinases and/or anti-VEGF antibodies and/or VEGF traps.

In some embodiments, methods of treating cancer comprising administering to a subject an FGFR1 ECD and/or FGFR1 ECD fusion molecule in combination with at least one additional therapeutic agent selected from docetaxel, paclitaxel, vincristine, carboplatin, cisplatin, oxaliplatin, doxorubicin, 5-fluorouracil (5-FU), leucovorin, pemetrexed, sorafenib, etoposide, topotecan, a VEGF antagonist, an anti-VEGF antibody, a VEGF trap, and bevacizumab are provided. In another example, methods of treating cancer comprising administering to a subject an FGFR1-ECD.339-Fc in combination with at least one additional therapeutic agent selected from docetaxel, paclitaxel, vincristine, carboplatin, cisplatin, oxaliplatin, doxorubicin, 5-fluorouracil (5-FU), leucovorin, pemetrexed, sorafenib, etoposide, topotecan, a VEGF antagonist, an anti-VEGF antibody, a VEGF trap, and bevacizumab are provided. In some embodiments, methods of treating cancer comprising administering to a subject an FGFR1-ECD.339-Fc and docetaxel are provided.

Pharmaceutical compositions comprising FGFR1 ECD and/or FGFR1 ECD fusion molecules (e.g., FGFR1-ECD.339-Fc) are administered in a therapeutically effective amount for the specific indication. The therapeutically effective amount is typically dependent on the weight of the subject being treated, his or her physical or health condition, the extensiveness of the condition to be treated, and/or the age of the subject being treated. In general, an FGFR1 ECD and/or FGFR1 ECD fusion molecule (e.g., FGFR1-ECD.339-Fc) is to be administered in an amount in the range of about 50 μg/kg body weight to about 100 mg/kg body weight per dose. Optionally, the FGFR1 ECD and/or FGFR1 ECD fusion molecule (e.g., FGFR1-ECD.339-Fc) can be administered in an amount in the range of about 100 μg/kg body weight to about 30 mg/kg body weight per dose. Further optionally, the FGFR1 ECD and/or FGFR1 ECD fusion molecule (e.g., FGFR1-ECD.339-Fc) can be administered in an amount in the range of about 0.5 mg/kg body weight to about 20 mg/kg body weight per dose. In certain embodiments, the FGFR1 ECD and/or FGFR1 ECD fusion molecule (e.g., FGFR1-ECD.339-Fc) is administered at a dose of about 5 mg/kg body weight to about 20 mg/kg body weight. In some embodiments, the FGFR1 ECD and/or FGFR1 ECD fusion molecule (e.g., FGFR1-ECD.339-Fc) is administered at a dose of about 10 mg/kg body weight to about 20 mg/kg body weight calculated using an extinction coefficient of 1.11 mL/mg*cm; see Table 1). In some embodiments, the FGFR1 ECD and/or FGFR1 ECD fusion molecule (e.g., FGFR1-ECD.339-Fc) is administered at a dose of about 10 mg/kg body weight, about 11 mg/kg body weight, about 12 mg/kg body weight, about 13 mg/kg body weight, about 14 mg/kg body weight, about 15 mg/kg body weight, about 16 mg/kg body weight, about 17 mg/kg body weight, about 18 mg/kg body weight, about 19 mg/kg body weight, or about 20 mg/kg body weight. In some embodiments, the FGFR1 fusion protein is administered at a dose of about 10 mg/kg body weight as calculated using an extinction coefficient of 1.11 mL/mg*cm. In other embodiments, the FGFR1 fusion protein is administered at a dose of about 20 mg/kg body weight as calculated using an extinction coefficient of 1.11 mL/mg*cm. The FGFR1 ECD and/or FGFR1 ECD fusion molecules may also be administered at ranges from one of the above doses to another. In some embodiments, dosages may be administered twice a week, weekly, every other week, at a frequency between weekly and every other week, every three weeks, every four weeks, or every month.

In certain embodiments, dosages of the FGFR1 ECD and/or FGFR1 ECD fusion molecules can be calculated in two ways depending on the extinction coefficient (EC) used. The extinction coefficient differs depending on whether the glycosylation of the proteins is taken into account. In one embodiment, the extinction coefficient based on the amino acid composition of FGFR1-ECD.339-Fc, for example, is 1.42 mL/mg*cm. In another embodiment, when the carbohydrate portion as well as the amino acid portion of FGFR1-ECD.339-Fc is accounted for, the extinction coefficient is 1.11 mL/mg*cm. Calculation of the FGFR1-ECD.339-Fc dose using an EC of 1.11 mL/mg*cm increases the calculated dose by 28%, as shown in Table 1. Although the doses calculated using the two extinction coefficients are different, the molar concentrations, or the actual amounts of drug administered, are identical. Unless otherwise noted, the doses disclosed herein are each calculated using the extinction coefficient that does not take account of glycosylation. How these dosages compare to those calculated using the extinction coefficient that takes account of glycosylation for FGFR1-ECD.339-Fc is shown in Table 1. As can be seen from Table 1, a dosage of 5 mg/kg using an EC of 1.11 mL/mg*cm herein corresponds to a dosage of about 3.9 mg/kg when calculated using an EC of 1.42 mL/mg*cm. A dosage of 10 mg/kg using an EC of 1.11 mL/mg*cm herein corresponds to a dosage of about 7.8 mg/kg when calculated using an EC of 1.42 mL/mg*cm. A dosage of 20 mg/kg using an EC of 1.11 mL/mg*cm herein corresponds to a dosage of about 15.6 mg/kg when calculated using an EC of 1.42 mL/mg*cm. As noted in the “Definitions” section above, measured numbers provided herein are approximate and encompass values having additional significant digits that are rounded off. For instance, 8 mg/kg encompasses values with two significant digits such as 7.6, 7.8, 8.0, 8.2, 8.4, and 8.45, each of which round to 8. Likewise, a value such as 16 mg/kg encompasses values with three significant digits that round to 16, such as, for example 15.6 and 16.4.

TABLE 1
Conversion of FGFR1-ECD.339-FC Dose

| Dose^{a} | Dose^{a} |
|---|---|
| EC = 1.11 mL/mg*cm | EC = 1.42 mL/mg*cm |
| 0.5 | 0.4 |
| 0.75 | 0.6 |
| 1.0 | 0.8 |
| 2.0 | 1.6 |
| 3.0 | 2.3 |
| 4.0 | 3.1 |
| 5.0 | 3.9 |
| 6.0 | 4.7 |
| 7.0 | 5.5 |
| 8.0 | 6.2 |
| 9.0 | 7.0 |
| 10.0 | 7.8 |
| 11.0 | 8.6 |
| 12.0 | 9.4 |
| 13.0 | 10.2 |
| 14.0 | 10.9 |
| 15.0 | 11.7 |
| 16.0 | 12.5 |
| 17.0 | 13.3 |
| 18.0 | 14.1 |
| 19.0 | 14.8 |
| 20.0 | 15.6 |
| 30.0 | 23.4 |
| Dose^{a} | Dose^{a} |
| EC = 1.42 mL/mg*cm | EC = 1.11 mL/mg*cm |
| 0.5 | 0.6 |
| 0.75 | 1.0 |
| 1.0 | 1.3 |
| 2.0 | 2.6 |
| 4.0 | 5.1 |
| 8.0 | 10.2 |
| 16.0 | 20.5 |

^{a}Doses shown in mg/kg.

The pharmaceutical compositions comprising FGFR1 ECDs, FGFR1 ECD fusion molecules, and/or at least one additional therapeutic agent can be administered as needed to subjects. In certain embodiments, an effective dose of a therapeutic molecule is administered to a subject one or more times. In various embodiments, an effective dose of a therapeutic molecule is administered to the subject at least once every two months, at least once a month, at least twice a month, once a week, twice a week, or three times a week. In various embodiments, an effective dose of a therapeutic molecule is administered to the subject for at least a week, at least a month, at least three months, at least six months, or at least a year.

In certain embodiments, the combined administration of an FGFR1 ECDs, FGFR1 ECD fusion molecule and at least one additional therapeutic agent includes concurrent administration, including simultaneous administration, using separate formulations or a single pharmaceutical formulation, as well as consecutive administration in any order. Optionally there is a time period while both (or all) active agents simultaneously exert their biological activities. Therapeutically effective amounts of therapeutic agents administered in combination with the FGFR1 ECD and/or FGFR1 ECD fusion molecule (e.g., FGFR1-ECD.339-Fc) will be at the physician's or veterinarian's discretion. Dosage administration and adjustment is done to achieve maximal management of the conditions to be treated. The dose will additionally depend on such factors as the type of therapeutic agent to be used, the specific patient being treated, the stage of the disease, and the desired aggressiveness of the treatment regime.

In any of the embodiments described herein, a therapeutic agent may be administered at a dosage approved by an agency responsible for approving therapeutic treatments, such as the Food and Drug Administration, or at the manufacturer's recommended dosage.

Routes of Administration and Carriers

In some embodiments, an FGFR1 ECD and/or FGFR1 ECD fusion molecule can be administered intravenously and/or subcutaneously. In some embodiments, an FGFR1 ECD and/or FGFR1 ECD fusion molecule can be administered by another route, such as intra-arterial, parenteral, intranasal, intramuscular, intracardiac, intraventricular, intratracheal, buccal, rectal, intraperitoneal, intradermal, topical, transdermal, or intrathecal, or otherwise by implantation or inhalation. In various embodiments, at least one additional therapeutic agent can be administered in vivo by a variety of routes, including intravenous, intra-arterial, subcutaneous, parenteral, intranasal, intramuscular, intracardiac, intraventricular, intratracheal, buccal, rectal, intraperitoneal, intradermal, topical, transdermal, and intrathecal, or otherwise by implantation or inhalation. Each of the subject compositions can be formulated alone or in combination into preparations in solid, semi-solid, liquid, or gaseous forms, such as tablets, capsules, powders, granules, ointments, solutions, suppositories, enemas, injections, inhalants, and aerosols.

In various embodiments, compositions comprising an FGFR1 ECD, FGFR1 ECD fusion molecule, and/or at least one additional therapeutic agent are provided in formulation with pharmaceutically acceptable carriers, a wide variety of which are known in the art (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7^{th} ed., Lippencott Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, carriers, and diluents, are available to the public. Moreover, various pharmaceutically acceptable auxiliary substances, such as pH adjusting and buffering agents, tonicity adjusting agents, stabilizers, wetting agents and the like, are also available to the public. Certain non-limiting exemplary carriers include saline, buffered saline, dextrose, water, glycerol, ethanol, and combinations thereof. In some embodiments, a therapeutic agent is formulated as the brand-name drug indicated above in the Definitions section, or a generic equivalent. In some embodiments, docetaxel is formulated as Taxotere® (Sanofi Aventis) or a generic equivalent.

In various embodiments, compositions comprising FGFR1 ECDs, FGFR1 ECD fusion molecules, and/or at least one additional therapeutic agent can be formulated for injection by dissolving, suspending, or emulsifying them in an aqueous or nonaqueous solvent, such as vegetable or other oils, synthetic aliphatic acid glycerides, esters of higher aliphatic acids, or propylene glycol; and if desired, with conventional additives such as solubilizers, isotonic agents, suspending agents, emulsifying agents, stabilizers and preservatives. In various embodiments, the compositions may be formulated for inhalation, for example, using pressurized acceptable propellants such as dichlorodifluoromethane, propane, nitrogen, and the like. The compositions may also be formulated, in various embodiments, into sustained release microcapsules, such as with biodegradable or non-biodegradable polymers. A non-limiting exemplary biodegradable formulation includes poly lactic acid-glycolic acid polymer. A non-limiting exemplary non-biodegradable formulation includes a polyglycerin fatty acid ester. Certain methods of making such formulations are described, for example, in EP 1 125 584 A1.

Pharmaceutical dosage packs comprising one or more containers, each containing one or more doses of an FGFR1 ECD, an FGFR1 ECD fusion molecule, and/or at least one additional therapeutic agent are also provided. In certain embodiments, a unit dosage is provided wherein the unit dosage contains a predetermined amount of a composition comprising an FGFR1 ECD, an FGFR1 ECD fusion molecule, and/or at least one additional therapeutic agent with or without one or more additional agents. In certain embodiments, such a unit dosage is supplied in single-use prefilled syringe for injection. In various embodiments, the composition contained in the unit dosage may comprise saline, sucrose, or the like; a buffer, such as phosphate, or the like; and/or be formulated within a stable and effective pH range. Alternatively, in certain embodiments, the composition may be provided as a lyophilized powder that can be reconstituted upon addition of an appropriate liquid, for example, sterile water. In certain embodiments, a composition comprises one or more substances that inhibit protein aggregation, including, but not limited to, sucrose and arginine. In certain embodiments, a composition of the invention comprises heparin and/or a proteoglycan.

In some embodiments, a dosage pack comprises instructions to determine whether a cancer comprises FGFR1 gene amplification, FGFR1 overexpression, FGFR3 gene amplification, FGFR3 overexpression, FGF2 overexpression, and/or FGF2 gene amplification, and/or overexpresses at least one, at least two, or three markers selected from DKK3, FGF18, and ETV4 prior to administering an FGFR1 ECD and/or an FGFR1 ECD fusion molecule. In some embodiments, FGFR1 is FGFR1IIIc. In some embodiments, FGFR3 is FGFR3IIIc. In some such embodiments, the instructions indicate that the presence of FGFR1 gene amplification, FGFR1 overexpression, FGFR3 gene amplification, FGFR3 overexpression, FGF2 overexpression, and/or FGF2 gene amplification, and/or overexpression of at least one, at least two, or three markers selected from DKK3, FGF18, and ETV4 in at least a portion of the cancer cells is indicative of therapeutic responsiveness to an FGFR1 ECD and/or an FGFR1 ECD fusion molecule.

In some embodiments, the instructions indicate that the presence of at least four copies of an FGFR1 gene in at least a portion of the cancer cells is indicative of therapeutic responsiveness to an FGFR1 ECD and/or an FGFR1 ECD fusion molecule. In some embodiments, the instructions indicate that the presence of at least four, at least six, at least eight, or at least ten copies of an FGFR1 gene in at least a portion of the cancer cells is indicative of therapeutic responsiveness to an FGFR1 ECD and/or an FGFR1 ECD fusion molecule. In some embodiments, the instructions indicate that a ratio of FGFR1 gene to chromosome 8 centromere of at least 2 in at least a portion of the cancer cells is indicative of therapeutic responsiveness to an FGFR1 ECD and/or an FGFR1 ECD fusion molecule. In some embodiments, the instructions indicate that a ratio of FGFR1 gene to chromosome 8 centromere of greater than 2 in at least a portion of the cancer cells is indicative of therapeutic responsiveness to an FGFR1 ECD and/or an FGFR1 ECD fusion molecule. In some embodiments, the instructions indicate that a ratio of FGFR1 gene to chromosome 8 centromere of at least 2.5, at least 3, at least 3.5, or at least 4 in at least a portion of the lung cancer cells is indicative of therapeutic responsiveness to an FGFR1 ECD and/or an FGFR1 ECD fusion molecule.

In some embodiments, the instructions indicate that the presence of at least four copies of an FGF2 gene in at least a portion of the cancer cells is indicative of therapeutic responsiveness to an FGFR1 ECD and/or an FGFR1 ECD fusion molecule. In some embodiments, the instructions indicate that the presence of at least four, at least six, at least eight, or at least ten copies of an FGF2 gene in at least a portion of the cancer cells is indicative of therapeutic responsiveness to an FGFR1 ECD and/or an FGFR1 ECD fusion molecule. In some embodiments, the instructions indicate that a ratio of FGF2 gene to chromosome 4 centromere of at least 2 in at least a portion of the cancer cells is indicative of therapeutic responsiveness to an FGFR1 ECD and/or an FGFR1 ECD fusion molecule. In some embodiments, the instructions indicate that a ratio of FGF2 gene to chromosome 4 centromere of greater than 2 in at least a portion of the cancer cells is indicative of therapeutic responsiveness to an FGFR1 ECD and/or an FGFR1 ECD fusion molecule. In some embodiments, the instructions indicate that a ratio of FGF2 gene to chromosome 4 centromere of at least 2.5, at least 3, at least 3.5, or at least 4 in at least a portion of the lung cancer cells is indicative of therapeutic responsiveness to an FGFR1 ECD and/or an FGFR1 ECD fusion molecule.

In some embodiments, the instructions indicate that the presence of at least four copies of an FGFR3 gene in at least a portion of the cancer cells is indicative of therapeutic responsiveness to an FGFR1 ECD and/or an FGFR1 ECD fusion molecule. In some embodiments, the instructions indicate that the presence of at least four, at least six, at least eight, or at least ten copies of an FGFR3 gene in at least a portion of the cancer cells is indicative of therapeutic responsiveness to an FGFR1 ECD and/or an FGFR1 ECD fusion molecule. In some embodiments, the instructions indicate that a ratio of FGFR3 gene to chromosome 8 centromere of at least 2 in at least a portion of the cancer cells is indicative of therapeutic responsiveness to an FGFR1 ECD and/or an FGFR1 ECD fusion molecule. In some embodiments, the instructions indicate that a ratio of FGFR3 gene to chromosome 8 centromere of greater than 2 in at least a portion of the cancer cells is indicative of therapeutic responsiveness to an FGFR1 ECD and/or an FGFR1 ECD fusion molecule. In some embodiments, the instructions indicate that a ratio of FGFR3 gene to chromosome 8 centromere of at least 2.5, at least 3, at least 3.5, or at least 4 in at least a portion of the lung cancer cells is indicative of therapeutic responsiveness to an FGFR1 ECD and/or an FGFR1 ECD fusion molecule.

The term “instructions,” as used herein includes, but is not limited to, labels, package inserts, instructions available in electronic form such as on a computer readable medium (e.g., a diskette, compact disk, or DVD), instructions available remotely such as over the internet, etc. A dosage pack is considered to include the instructions when the dosage pack provides access to the instructions, a link to the instructions (such as a uniform resource locator, or url), or other mechanism for obtaining a copy of the instructions (such as a return reply card, a physical address from which instructions may be requested, an e-mail address from which instructions may be requested, a phone number that may be called to obtain instructions, etc.).

FGFR1 ECDs and FGFR1 ECD Fusion Molecules

Nonlimiting exemplary FGFR1 ECDs include full-length FGFR1 ECDs, FGFR1 ECD fragments, and FGFR1 ECD variants. FGFR1 ECDs may include or lack a signal peptide. Exemplary FGFR1 ECDs include, but are not limited to, FGFR1 ECDs having amino acid sequences selected from SEQ ID NOs.: 1, 2, 3, and 4.

Non-limiting exemplary FGFR1 ECD fragments include human FGFR1 ECD ending at amino acid 339 (counting from the first amino acid of the mature form, without the signal peptide). In some embodiments, an FGFR1 ECD fragment ends at an amino acid between amino acid 339 and amino acid 360 (counting from the first amino acid of the mature form, without the signal peptide). Exemplary FGFR1 ECD fragments include, but are not limited to, FGFR1 ECD fragments having amino acid sequences selected from SEQ ID NOs.: 3 and 4.

In some embodiments, an FGFR1 ECD comprises a sequence selected from SEQ ID NOs: 1 to 4. In some embodiments, an FGFR1 ECD consists of a sequence selected from SEQ ID NOs: 1 to 4. When an FGFR1 ECD “consists of” a sequence selected from SEQ ID NOs: 1 to 4, the FGFR1 ECD may or may not contain various post-translational modifications, such as glycosylation and sialylation. In other words, when an FGFR1 ECD consists of a particular amino acid sequence, it does not contain additional amino acids in the contiguous amino acid sequence, but may contain modifications to amino acid side chains, the N-terminal amino group, and/or the C-terminal carboxy group.

In some embodiments, an FGFR1 ECD fusion molecule comprises a signal peptide. In some embodiments, an FGFR1 ECD fusion molecule lacks a signal peptide. In some embodiments, the FGFR1 ECD portion of an FGFR1 ECD fusion molecule comprises a sequence selected from SEQ ID NOs: 1 to 4. In some embodiments, the FGFR1 ECD portion of an FGFR1 ECD fusion molecule consists of a sequence selected from SEQ ID NOs: 1 to 4. When an FGFR1 ECD portion of an FGFR1 ECD fusion molecule “consists of” a sequence selected from SEQ ID NOs: 1 to 4, the FGFR1 ECD portion of an FGFR1 ECD fusion molecule may or may not contain various post-translational modifications, such as glycosylation and sialylation. In other words, when an FGFR1 ECD portion of an FGFR1 ECD fusion molecule consists of a particular amino acid sequence, it does not contain additional amino acids from FGFR1 in the contiguous amino acid sequence, but may contain modifications to amino acid side chains, the N-terminal amino group, and/or the C-terminal carboxy group. Further, because the FGFR1 ECD is linked to a fusion molecule, there may be additional amino acids at the N- and/or C-terminus of the FGFR1 ECD, but those amino acids are not from the FGFR1 sequence, but may be from, for example, a linker sequence, or a fusion partner sequence.

In some embodiments, the fusion partner portion of an FGFR1 ECD fusion molecule is selected from Fc, albumin, and polyethylene glycol. Nonlimiting exemplary fusion partners are discussed herein.

Fusion Partners and Conjugates

As discussed herein, an FGFR1 ECD may be combined with at least one fusion partner, resulting in an FGFR1 ECD fusion molecule. These fusion partners may facilitate purification, and the FGFR1 ECD fusion molecules may show an increased half-life in vivo. Suitable fusion partners of an FGFR1 ECD include, for example, polymers, such as water soluble polymers, the constant domain of immunoglobulins; all or part of human serum albumin (HSA); fetuin A; fetuin B; a leucine zipper domain; a tetranectin trimerization domain; mannose binding protein (also known as mannose binding lectin), for example, mannose binding protein 1; and an Fc region, as described herein and further described in U.S. Pat. No. 6,686,179. Nonlimiting exemplary FGFR1 ECD fusion molecules are described, e.g., in U.S. Pat. No. 7,678,890.

An FGFR1 ECD fusion molecule may be prepared by attaching polyaminoacids or branch point amino acids to the FGFR1 ECD. For example, the polyaminoacid may be a carrier protein that serves to increase the circulation half life of the FGFR1 ECD (in addition to the advantages achieved via a fusion molecule). For the therapeutic purpose of the present invention, such polyaminoacids should ideally be those that have or do not create neutralizing antigenic responses, or other adverse responses. Such polyaminoacids may be chosen from serum albumin (such as HSA), an additional antibody or portion thereof, for example the Fc region, fetuin A, fetuin B, leucine zipper nuclear factor erythroid derivative-2 (NFE2), neuroretinal leucine zipper, tetranectin, or other polyaminoacids, for example, lysines. As described herein, the location of attachment of the polyaminoacid may be at the N terminus or C terminus, or other places in between, and also may be connected by a chemical linker moiety to the selected molecule.

Polymers

Polymers, for example, water soluble polymers, may be useful as fusion partners to reduce precipitation of the FGFR1 ECD fusion molecule in an aqueous environment, such as typically found in a physiological environment. Polymers employed in the invention will be pharmaceutically acceptable for the preparation of a therapeutic product or composition.

Suitable, clinically acceptable, water soluble polymers include, but are not limited to, polyethylene glycol (PEG), polyethylene glycol propionaldehyde, copolymers of ethylene glycol/propylene glycol, monomethoxy-polyethylene glycol, carboxymethylcellulose, dextran, polyvinyl alcohol (PVA), polyvinyl pyrrolidone, poly-1,3-dioxolane, poly-1,3,6-trioxane, ethylene/maleic anhydride copolymer, poly (β-amino acids) (either homopolymers or random copolymers), poly(n-vinyl pyrrolidone) polyethylene glycol, polypropylene glycol homopolymers (PPG) and other polyakylene oxides, polypropylene oxide/ethylene oxide copolymers, polyoxyethylated polyols (POG) (e.g., glycerol) and other polyoxyethylated polyols, polyoxyethylated sorbitol, or polyoxyethylated glucose, colonic acids or other carbohydrate polymers, Ficoll, or dextran and mixtures thereof.

As used herein, polyethylene glycol (PEG) is meant to encompass any of the forms that have been used to derivatize other proteins, such as mono-(C₁-C₁₀) alkoxy- or aryloxy-polyethylene glycol. Polyethylene glycol propionaldehyde may have advantages in manufacturing due to its stability in water.

Polymers used herein, for example water soluble polymers, may be of any molecular weight and may be branched or unbranched. In some embodiments, the polymers have an average molecular weight of between about 2 kDa to about 100 kDa (the term “about” indicating that in preparations of a polymer, some molecules will weigh more, some less, than the stated molecular weight). The average molecular weight of each polymer may be between about 5 kDa and about 50 kDa, or between about 12 kDa and about 25 kDa. Generally, the higher the molecular weight or the more branches, the higher the polymer:protein ratio. Other sizes may also be used, depending on the desired therapeutic profile; for example, the duration of sustained release; the effects, if any, on biological activity; the ease in handling; the degree or lack of antigenicity; and other known effects of a polymer on an FGFR1 ECD.

Polymers employed in the present invention are typically attached to an FGFR1 ECD with consideration of effects on functional or antigenic domains of the polypeptide. In general, chemical derivatization may be performed under any suitable condition used to react a protein with an activated polymer molecule. Activating groups which can be used to link the polymer to the active moieties include sulfone, maleimide, sulfhydryl, thiol, triflate, tresylate, azidirine, oxirane, and 5-pyridyl.

Polymers of the invention are typically attached to a heterologous polypeptide at the alpha (a) or epsilon (E) amino groups of amino acids or a reactive thiol group, but it is also contemplated that a polymer group could be attached to any reactive group of the protein that is sufficiently reactive to become attached to a polymer group under suitable reaction conditions. Thus, a polymer may be covalently bound to an FGFR1 ECD via a reactive group, such as a free amino or carboxyl group. The amino acid residues having a free amino group may include lysine residues and the N-terminal amino acid residue. Those having a free carboxyl group may include aspartic acid residues, glutamic acid residues, and the C-terminal amino acid residue. Those having a reactive thiol group include cysteine residues.

Methods for preparing fusion molecules conjugated with polymers, such as water soluble polymers, will each generally involve (a) reacting an FGFR1 ECD with a polymer under conditions whereby the polypeptide becomes attached to one or more polymers and (b) obtaining the reaction product. Reaction conditions for each conjugation may be selected from any of those known in the art or those subsequently developed, but should be selected to avoid or limit exposure to reaction conditions such as temperatures, solvents, and pH levels that would inactivate the protein to be modified. In general, the optimal reaction conditions for the reactions will be determined case-by-case based on known parameters and the desired result. For example, the larger the ratio of polymer:polypeptide conjugate, the greater the percentage of conjugated product. The optimum ratio (in terms of efficiency of reaction in that there is no excess unreacted polypeptide or polymer) may be determined by factors such as the desired degree of derivatization (e.g., mono-, di-, tri-, etc.), the molecular weight of the polymer selected, whether the polymer is branched or unbranched and the reaction conditions used. The ratio of polymer (for example, PEG) to a polypeptide will generally range from 1:1 to 100:1. One or more purified conjugates may be prepared from each mixture by standard purification techniques, including among others, dialysis, salting-out, ultrafiltration, ion-exchange chromatography, gel filtration chromatography, and electrophoresis.

One may specifically desire an N-terminal chemically modified FGFR1 ECD. One may select a polymer by molecular weight, branching, etc., the proportion of polymers to FGFR1 ECD molecules in the reaction mix, the type of reaction to be performed, and the method of obtaining the selected N-terminal chemically modified FGFR1 ECD. The method of obtaining the N-terminal chemically modified FGFR1 ECD preparation (separating this moiety from other monoderivatized moieties if necessary) may be by purification of the N-terminal chemically modified FGFR1 ECD material from a population of chemically modified protein molecules.

Selective N-terminal chemical modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N terminus with a carbonyl group-containing polymer is achieved. For example, one may selectively attach a polymer to the N terminus of the protein by performing the reaction at a pH that allows one to take advantage of the pKa differences between the ε-amino group of the lysine residues and that of the α-amino group of the N-terminal residue of the protein. By such selective derivatization, attachment of a polymer to a protein is controlled: the conjugation with the polymer takes place predominantly at the N terminus of the protein and no significant modification of other reactive groups, such as the lysine side chain amino groups, occurs. Using reductive alkylation, the polymer may be of the type described above and should have a single reactive aldehyde for coupling to the protein. Polyethylene glycol propionaldehyde, containing a single reactive aldehyde, may also be used.

In one embodiment, the present invention contemplates the chemically derivatized FGFR1 ECD to include mono- or poly-(e.g., 2-4) PEG moieties. Pegylation may be carried out by any of the pegylation reactions available. Methods for preparing a pegylated protein product will generally include (a) reacting a polypeptide with polyethylene glycol (such as a reactive ester or aldehyde derivative of PEG) under conditions whereby the protein becomes attached to one or more PEG groups; and (b) obtaining the reaction product(s). In general, the optimal reaction conditions will be determined case by case based on known parameters and the desired result.

There are a number of PEG attachment methods known in the art. See, for example, EP 0 401 384; Malik et al., Exp. Hematol., 20:1028-1035 (1992); Francis, Focus on Growth Factors, 3(2):4-10 (1992); EP 0 154 316; EP 0 401 384; WO 92/16221; WO 95/34326; and the other publications cited herein that relate to pegylation.

Pegylation may be carried out, e.g., via an acylation reaction or an alkylation reaction with a reactive polyethylene glycol molecule. Thus, protein products according to the present invention include pegylated proteins wherein the PEG group(s) is (are) attached via acyl or alkyl groups. Such products may be mono-pegylated or poly-pegylated (for example, those containing 2-6 or 2-5 PEG groups). The PEG groups are generally attached to the protein at the α- or ε-amino groups of amino acids, but it is also contemplated that the PEG groups could be attached to any amino group attached to the protein that is sufficiently reactive to become attached to a PEG group under suitable reaction conditions.

Pegylation by acylation generally involves reacting an active ester derivative of polyethylene glycol (PEG) with an FGFR1 ECD. For acylation reactions, the polymer(s) selected typically have a single reactive ester group. Any known or subsequently discovered reactive PEG molecule may be used to carry out the pegylation reaction. An example of a suitable activated PEG ester is PEG esterified to N-hydroxysuccinimide (NHS). As used herein, acylation is contemplated to include, without limitation, the following types of linkages between the therapeutic protein and a polymer such as PEG: amide, carbamate, urethane, and the like, see for example, Chamow, Bioconjugate Chem., 5:133-140 (1994). Reaction conditions may be selected from any of those currently known or those subsequently developed, but should avoid conditions such as temperature, solvent, and pH that would inactivate the polypeptide to be modified.

Pegylation by acylation will generally result in a poly-pegylated protein. The connecting linkage may be an amide. The resulting product may be substantially only (e.g., >95%) mono-, di-, or tri-pegylated. However, some species with higher degrees of pegylation may be formed in amounts depending on the specific reaction conditions used. If desired, more purified pegylated species may be separated from the mixture (particularly unreacted species) by standard purification techniques, including among others, dialysis, salting-out, ultrafiltration, ion-exchange chromatography, gel filtration chromatography, and electrophoresis.

Pegylation by alkylation generally involves reacting a terminal aldehyde derivative of PEG with a polypeptide in the presence of a reducing agent. For the reductive alkylation reaction, the polymer(s) selected should have a single reactive aldehyde group. An exemplary reactive PEG aldehyde is polyethylene glycol propionaldehyde, which is water stable, or mono C₁-C₁₀ alkoxy or aryloxy derivatives thereof, see for example, U.S. Pat. No. 5,252,714.

Markers

Moreover, FGFR1 ECDs of the present invention may be fused to marker sequences, such as a peptide that facilitates purification of the fused polypeptide. The marker amino acid sequence may be a hexa-histidine peptide such as the tag provided in a pQE vector (Qiagen, Mississauga, Ontario, Canada), among others, many of which are commercially available. As described in Gentz et al., Proc. Natl. Acad. Sci. 86:821-824 (1989), for instance, hexa-histidine provides for convenient purification of the fusion protein. Another peptide tag useful for purification, the hemagglutinin (HA) tag, corresponds to an epitope derived from the influenza HA protein. (Wilson et al., Cell 37:767 (1984)). Any of these above fusions may be engineered using the FGFR1 ECDs described herein.

Oligomerization Domain Fusion Partners

In various embodiments, oligomerization offers some functional advantages to a fusion protein, including, but not limited to, multivalency, increased binding strength, and the combined function of different domains. Accordingly, in some embodiments, a fusion partner comprises an oligomerization domain, for example, a dimerization domain. Exemplary oligomerization domains include, but are not limited to, coiled-coil domains, including alpha-helical coiled-coil domains; collagen domains; collagen-like domains; and certain immunoglobulin domains. Exemplary coiled-coil polypeptide fusion partners include, but are not limited to, the tetranectin coiled-coil domain; the coiled-coil domain of cartilage oligomeric matrix protein; angiopoietin coiled-coil domains; and leucine zipper domains. Exemplary collagen or collagen-like oligomerization domains include, but are not limited to, those found in collagens, mannose binding lectin, lung surfactant proteins A and D, adiponectin, ficolin, conglutinin, macrophage scavenger receptor, and emilin.

Antibody Fc Immunoglobulin Domain Fusion Partners

Many Fc domains that may be used as fusion partners are known in the art. In some embodiments, a fusion partner is an Fc immunoglobulin domain. An Fc fusion partner may be a wild-type Fc found in a naturally occurring antibody, a variant thereof, or a fragment thereof. Non-limiting exemplary Fc fusion partners include Fcs comprising a hinge and the CH2 and CH3 constant domains of a human IgG, for example, human IgG1, IgG2, IgG3, or IgG4. Additional exemplary Fc fusion partners include, but are not limited to, human IgA and IgM. In some embodiments, an Fc fusion partner comprises a C237S mutation, for example, in an IgG1 (see, for example, SEQ ID NO: 8). In some embodiments, an Fc fusion partner comprises a hinge, CH2, and CH3 domains of human IgG2 with a P331S mutation, as described in U.S. Pat. No. 6,900,292. Certain exemplary Fc domain fusion partners are shown in SEQ ID NOs: 8 to 10.

Albumin Fusion Partners and Albumin-Binding Molecule Fusion Partners

In some embodiments, a fusion partner is an albumin. Exemplary albumins include, but are not limited to, human serum album (HSA) and fragments of HSA that are capable of increasing the serum half-life or bioavailability of the polypeptide to which they are fused. In some embodiments, a fusion partner is an albumin-binding molecule, such as, for example, a peptide that binds albumin or a molecule that conjugates with a lipid or other molecule that binds albumin. In some embodiments, a fusion molecule comprising HSA is prepared as described, e.g., in U.S. Pat. No. 6,686,179.

Exemplary Attachment of Fusion Partners

The fusion partner may be attached, either covalently or non-covalently, to the N terminus or the C terminus of the FGFR1 ECD. The attachment may also occur at a location within the FGFR1 ECD other than the N terminus or the C terminus, for example, through an amino acid side chain (such as, for example, the side chain of cysteine, lysine, serine, or threonine).

In either covalent or non-covalent attachment embodiments, a linker may be included between the fusion partner and the FGFR1 ECD. Such linkers may be comprised of at least one amino acid or chemical moiety. Exemplary methods of covalently attaching a fusion partner to an FGFR1 ECD include, but are not limited to, translation of the fusion partner and the FGFR1 ECD as a single amino acid sequence and chemical attachment of the fusion partner to the FGFR1 ECD. When the fusion partner and an FGFR1 ECD are translated as single amino acid sequence, additional amino acids may be included between the fusion partner and the FGFR1 ECD as a linker. In some embodiments, the linker is selected based on the polynucleotide sequence that encodes it, to facilitate cloning the fusion partner and/or FGFR1 ECD into a single expression construct (for example, a polynucleotide containing a particular restriction site may be placed between the polynucleotide encoding the fusion partner and the polynucleotide encoding the FGFR1 ECD, wherein the polynucleotide containing the restriction site encodes a short amino acid linker sequence). When the fusion partner and the FGFR1 ECD are covalently coupled by chemical means, linkers of various sizes may typically be included during the coupling reaction.

Exemplary methods of non-covalently attaching a fusion partner to an FGFR1 ECD include, but are not limited to, attachment through a binding pair. Exemplary binding pairs include, but are not limited to, biotin and avidin or streptavidin, an antibody and its antigen, etc.

Co-Translational and Post-Translational Modifications

The invention encompasses administration of FGFR1 ECDs and FGFR1 ECD fusion molecules that are differentially modified during or after translation, for example by glycosylation, acetylation, phosphorylation, amidation, derivatization by known protecting/blocking groups, proteolytic cleavage, or linkage to an antibody molecule or other cellular ligand. Any of numerous chemical modifications may be carried out by known techniques, including, but not limited to, specific chemical cleavage by cyanogen bromide, trypsin, chymotrypsin, papain, V8 protease; NABH₄; acetylation; formylation; oxidation; reduction; and/or metabolic synthesis in the presence of tunicamycin.

Additional post-translational modifications encompassed by the invention include, for example, for example, N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of prokaryotic host cell expression. A nonlimiting discussion of various post-translational modifications of FGFR1 ECDs and FGFR1 ECD fusion molecules can be found, e.g., in U.S. Pat. No. 7,678,890.

FGFR1 ECD and FGFR1 ECD Fusion Molecule Expression and Production Vectors

Vectors comprising polynucleotides that encode FGFR1 ECDs are provided. Vectors comprising polynucleotides that encode FGFR1 ECD fusion molecules are also provided. Such vectors include, but are not limited to, DNA vectors, phage vectors, viral vectors, retroviral vectors, etc.

In some embodiments, a vector is selected that is optimized for expression of polypeptides in CHO or CHO-derived cells. Exemplary such vectors are described, e.g., in Running Deer et al., Biotechnol. Frog. 20:880-889 (2004).

In some embodiments, a vector is chosen for in vivo expression of FGFR1 ECDs and/or FGFR1 ECD fusion molecules in animals, including humans. In some such embodiments, expression of the polypeptide is under the control of a promoter that functions in a tissue-specific manner. For example, liver-specific promoters are described, e.g., in PCT Publication No. WO 2006/076288. A nonlimiting discussion of various expression vectors can be found, e.g., in U.S. Pat. No. 7,678,890.

Host Cells

In various embodiments, FGFR1 ECDs or FGFR1 ECD fusion molecules may be expressed in prokaryotic cells, such as bacterial cells; or in eukaryotic cells, such as fungal cells, plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO—S and DG44 cells; and NSO cells. In some embodiments, a particular eukaryotic host cell is selected based on its ability to make certain desired post-translational modifications to the FGFR1 ECDs or FGFR1 ECD fusion molecules. For example, in some embodiments, CHO cells produce FGFR1 ECDs and/or FGFR1 ECD fusion molecules that have a higher level of sialylation than the same polypeptide produced in 293 cells.

Introduction of a nucleic acid into a desired host cell may be accomplished by any method known in the art, including but not limited to, calcium phosphate transfection, DEAE-dextran mediated transfection, cationic lipid-mediated transfection, electroporation, transduction, infection, etc. Nonlimiting exemplary methods are described, e.g., in Sambrook et al., Molecular Cloning, A Laboratory Manual, 3^{rd} ed. Cold Spring Harbor Laboratory Press (2001). Nucleic acids may be transiently or stably transfected in the desired host cells, according to methods known in the art. A nonlimiting discussion of host cells and methods of polypeptides in host cells can be found, e.g., in U.S. Pat. No. 7,678,890.

In some embodiments, a polypeptide may be produced in vivo in an animal that has been engineered or transfected with a nucleic acid molecule encoding the polypeptide, according to methods known in the art.

Purification of FGFR1 ECD Polypeptides

FGFR1 ECDs or FGFR1 ECD fusion molecules may be purified by various methods known in the art. Such methods include, but are not limited to, the use of affinity matrices or hydrophobic interaction chromatography. Suitable affinity ligands include any ligands of the FGFR1 ECD or of the fusion partner. Suitable affinity ligands in the case of an antibody that binds FGFR1 include, but are not limited to, FGFR1 itself and fragments thereof. Further, a Protein A, Protein G, Protein A/G, or an antibody affinity column may be used to bind to an Fc fusion partner to purify an FGFR1 ECD fusion molecule. Antibodies to FGFR1 ECD may also be used to purify FGFR1 ECD or FGFR1 ECD fusion molecules. Hydrophobic interactive chromatography, for example, a butyl or phenyl column, may also suitable for purifying some polypeptides. Many methods of purifying polypeptides are known in the art. A nonlimiting discussion of various methods of purifying polypepides can be found, e.g., in U.S. Pat. No. 7,678,890.

Methods of Identifying Patients Who would Benefit from FGFR1 ECDs and/or FGFR1 ECD Fusion Molecules

In some embodiments, methods of identifying patients with cancer who may benefit from administration of an FGFR1 ECD or FGFR1 ECD fusion molecule are provided. In some such embodiments, the method comprises determining whether at least a portion of the cancer cells comprise FGFR1 gene amplification, FGFR1 overexpression, FGFR3 gene amplification, FGFR3 overexpression, FGF2 overexpression, and/or FGF2 gene amplification in a sample obtained from the subject. In some embodiments, FGFR1 gene amplification, FGFR1 overexpression, FGFR3 gene amplification, FGFR3 overexpression, FGF2 overexpression, and/or FGF2 gene amplification is indicative of therapeutic responsiveness by the cancer to an FGFR1 ECD or FGFR1 ECD fusion molecule. In some embodiments, a sample is taken from a patient having or suspected of having cancer. A finding of FGFR1 gene amplification, FGFR1 overexpression, FGFR3 gene amplification, FGFR3 overexpression, FGF2 overexpression, and/or FGF2 gene amplification in at least a portion of the cancer cells indicates that the patient having or suspected of having cancer may benefit from an FGFR1 ECD or FGFR1 ECD fusion molecule therapy. In some embodiments, the patient has or is suspected of having lung cancer.

In some embodiments, the method comprises determining whether at least a portion of the cancer cells comprise overexpression of at least one, at least two, at least three, or at least four markers selected from FGFR1, FGFR3 (such as FGFR3IIIc), FGF2, DKK3, FGF18, and ETV4 in a sample obtained from the subject. In some embodiments, the overexpression is mRNA overexpression. In some embodiments, the overexpression is protein overexpression. In some embodiments, FGFR1, FGFR3 (such as FGFR3IIIc), FGF2, DKK3, FGF18, and/or ETV4 overexpression is indicative of therapeutic responsiveness by the cancer to an FGFR1 ECD or FGFR1 ECD fusion molecule. In some embodiments, a sample is taken from a patient having or suspected of having cancer. A finding of FGFR1, FGFR3 (such as FGFR3IIIc), FGF2, DKK3, FGF18, and/or ETV4 overexpression in at least a portion of the cancer cells indicates that the patient having or suspected of having cancer may benefit from an FGFR1 ECD or FGFR1 ECD fusion molecule therapy. In some embodiments, FGFR1 is FGFR1IIIc. In some embodiments, FGFR3 is FGFR3IIIc. In some embodiments, the patient has or is suspected of having a cancer selected from breast cancer, ovarian cancer, prostate cancer, and carcinoid cancer.

In some embodiments, methods of identifying breast cancer patients with cancer who may benefit from administration of an FGFR1 ECD or FGFR1 ECD fusion molecule comprise determining whether the breast cancer is estrogen receptor (ER) positive, progesterone (PR) positive, or ER positive and PR positive. In some embodiments, methods of identifying breast cancer patients with cancer who may benefit from administration of an FGFR1 ECD or FGFR1 ECD fusion molecule comprise determining whether the breast cancer is HER2 positive or HER2 negative. In some embodiments, a method comprises determining whether the cancer is p95HER2 positive.

In some embodiments, methods of identifying ovarian cancer patients with cancer who may benefit from administration of an FGFR1 ECD or FGFR1 ECD fusion molecule comprise determining whether the ovarian cancer is estrogen receptor (ER) positive, progesterone (PR) positive, or ER positive and PR positive.

In some embodiments, gene amplification and/or expression is determined by a laboratory. A laboratory may be a hospital laboratory or a laboratory independent of a hospital. In some embodiments, following a determination of gene amplification and/or expression, the results of the determination are communicated to a medical professional. In some such embodiments, the results are communicated for the purpose of determining whether a patient should benefit from, or be responsive to, an FGFR1 ECD or FGFR1 ECD fusion molecule therapy. In some embodiments, medical professionals include, but are not limited to, doctors, nurses, hospital administration and staff, etc.

Any suitable method of determining gene amplification may be used in the methods described herein. Nonlimiting exemplary such methods include fluorescence in situ hybridization (FISH; see, e.g., Monni et al. (2001) PNAS 98: 5711-5716), array comparative genomic hybridization (aCGH), DNA microarrays (see, e.g., Carter et al. (2007) Nat. Genet. 39: S16-21), spectral karyotyping (SKY; see, e.g. Liyanage et al. (1996) Nat. Genet. 14: 312-5), real-time quantitative PCR (see, e.g., Dhaene et al. (2010) Methods 50: 262-270), southern blotting, and sequencing, including, but not limited to, high-throughput sequencing (HTS; see, e.g. Medvedev et al. (2010) Genome Res. 20: 1613-22), and next generation sequencing technologies such as RNA-seq, also called “Whole Transcriptome Shotgun Sequencing” (“WTSS”), Applied Biosystems SOLiD™ System, Illumina (Solexa) sequencing, Ion semiconductor sequencing, DNA nanoball sequencing, Helioscope™ single molecule sequencing, Single Molecule SMRT™ sequencing, Single Molecule real time (RNAP) sequencing, Nanopore DNA sequencing, VisiGen Biotechnologies approach, and 454 pyrosequencing.

Fluorescence in situ hybridization (FISH) is a cytogenetic technique to detect and localize the presence or absence of specific DNA sequences on chromosomes. In some embodiments, FISH uses fluorescent probes to detect certain regions of chromosomes in a sequence-specific manner. Thus, in some embodiments, to detect gene amplification in cancer using FISH, in some embodiments, a fluorescent probe is developed that binds specifically to the gene of interest, such as, without limitation, the FGFR1 gene, FGFR3 gene, FGF2 gene, or HER2 gene. In some such embodiments, this gene specific probe is hybridized to a cancer sample and the copy number determined by counting the number of fluorescent signals present per cell using fluorescence microscopy. For a normal diploid cell, the majority of genes will have a copy number of two (exceptions exist when the gene is present on one of the sex chromosomes rather than an autosome or the cell is undergoing division and the genome replicated). If more than two signals are detected in a cell, in certain instances, the gene may be amplified.

Dual color FISH may also be used for assessing gene amplification in cancer. In some embodiments, a reference probe that binds to the centromere region of the chromosome on which the gene of interest is located can be used as a control. In some instances, the centromere (CEN) region of a chromosome is considered to be genomically stable and is therefore assumed to be representative of the entire chromosome. CEN copy number can therefore, in some embodiments, assist in distinguishing focal gene amplification from increased gene copy number resulting from polysomy (≧3 copies of the chromosome centromere) of the chromosome. Gene amplification can be distinguished from polysomy, in some embodiments, by calculating the ratio the signal from the gene-of-interest probe/signal from the centromere probe. For a normal diploid cell, where the gene of interest in located on an autosome, this ratio is typically 1. In some embodiments, a ratio of >1 is indicative of gene amplification. In some embodiments, a probe to a chromosomal reference gene can be used in place of, or in addition to, a centromere probe (see, e.g., Tse et al. (2011) J. Clin. Oncol. 29: 4168-74). In some embodiments, the selected reference gene is on chromosome 8 or chromosome 4. In some embodiments, the reference gene is located close to the centromere of chromosome 8 or chromosome 4. In some embodiments, the reference sequence comprises non-coding DNA on chromosome 8 or chromosome 4.

In some embodiments, FISH allows the determination of multiple parameters of gene amplification, including, but not limited to, the fraction of cells with an amplified gene, the amplification levels within various subpopulations of cells, and the amplification pattern within a cell (for example, a clustered signal versus multiple scattered signals). In some embodiments, the ratio of the copy number of the gene of interest to the centromere reference for each cancer cell is determined. In some such embodiments, the mean ratio for a particular sample or subset of cells in a sample is then calculated. A mean ratio of greater than two is generally considered to indicate gene amplification, whereas signals between 1.5 to 2 may indicate low-level amplification. In some embodiments, cells that have a greater copy number of the gene of interest than a reference control probe are considered amplified (see, e.g., Kobayashi et al. (2002) Hum. Pathol. 33: 21-8; and Kunitomo et al. (2002) Pathol. Int. 52: 451-7). In some embodiments, single-color FISH is used to determine the copy number of a gene of interest without a chromosomal reference probe control. In some such embodiments, four or more copies of the gene per nucleus is considered to be gene amplification (see, e.g., Couturier et al. (2000) Mod. Pathol. 13: 1238-43; Jacobs et al. (1999) J. Clin. Oncol. 17: 1974-82; Wang et al. (2000) J. Clin. Pathol. 53: 374-81).

Any suitable method of determining protein expression (for example, FGFR1 (including FGFR1IIIc), FGFR3 (including FGFR3IIIc), FGF2, DKK3, FGF18, ETV4, ER, PR, and/or HER2 expression) may be used. In certain embodiments, the expression of proteins in a sample is examined using immunohistochemistry (“IHC”) and staining protocols Immunohistochemical staining of tissue sections has been shown to be a reliable method of assessing or detecting presence of proteins in a sample. Immunohistochemistry techniques utilize an antibody to probe and visualize cellular antigens in situ, generally by chromogenic or fluorescent methods. Nonlimiting exemplary methods of determining protein expression also include dextran-coated charcoal (DCC) or ligand-binding assay (LBA), enzyme immunoassay (EIA), enzyme-linked immunosorbent assay (ELISA), and flow cytometry.

The tissue sample may be fixed (i.e. preserved) by conventional methodology (See e.g., “Manual of Histological Staining Method of the Armed Forces Institute of Pathology,” 3^{rd} edition (1960) Lee G. Luna, H T (ASCP) Editor, The Blakston Division McGraw-Hill Book Company, New York; The Armed Forces Institute of Pathology Advanced Laboratory Methods in Histology and Pathology (1994) Ulreka V. Mikel, Editor, Armed Forces Institute of Pathology, American Registry of Pathology, Washington, D.C.). One of skill in the art will appreciate that the choice of a fixative is determined by the purpose for which the sample is to be histologically stained or otherwise analyzed. One of skill in the art will also appreciate that the length of fixation depends upon the size of the tissue sample and the fixative used. By way of example, neutral buffered formalin, Bouin's or paraformaldehyde, may be used to fix a sample.

Generally, the sample is first fixed and is then dehydrated through an ascending series of alcohols, infiltrated and embedded with paraffin or other sectioning media so that the tissue sample may be sectioned. Alternatively, one may section the tissue and fix the sections obtained. By way of example, the tissue sample may be embedded and processed in paraffin by conventional methodology (See e.g., “Manual of Histological Staining Method of the Armed Forces Institute of Pathology”, supra). Examples of paraffin that may be used include, but are not limited to, Paraplast, Broloid, and Tissuemay. Once the tissue sample is embedded, the sample may be sectioned by a microtome or the like (See e.g., “Manual of Histological Staining Method of the Armed Forces Institute of Pathology”, supra). By way of example for this procedure, sections may range from about three microns to about five microns in thickness. Once sectioned, the sections may be attached to slides by several standard methods. Examples of slide adhesives include, but are not limited to, silane, gelatin, poly-L-lysine and the like. By way of example, the paraffin embedded sections may be attached to positively charged slides and/or slides coated with poly-L-lysine.

If paraffin has been used as the embedding material, the tissue sections are generally deparaffinized and rehydrated to water. The tissue sections may be deparaffinized by several conventional standard methodologies. For example, xylenes and a gradually descending series of alcohols may be used (See e.g., “Manual of Histological Staining Method of the Armed Forces Institute of Pathology”, supra). Alternatively, commercially available deparaffinizing non-organic agents such as Hemo-De7 (CMS, Houston, Tex.) may be used.

In some embodiments, subsequent to the sample preparation, a tissue section may be analyzed using IHC. IHC may be performed in combination with additional techniques such as morphological staining and/or fluorescence in-situ hybridization. Two general methods of IHC are available; direct and indirect assays. According to the first assay, binding of antibody to the target antigen is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody. Where the secondary antibody is conjugated to an enzymatic label, a chromogenic or fluorogenic substrate is added to provide visualization of the antigen. Signal amplification occurs because several secondary antibodies may react with different epitopes on the primary antibody.

The primary and/or secondary antibody used for immunohistochemistry typically will be labeled with a detectable moiety. Numerous labels are available which can be generally grouped into the following categories: (a) Radioisotopes, such as ³⁵S, ¹⁴C, ¹²⁵I, ³H, and ¹³¹I. The antibody can be labeled with the radioisotope using the techniques described in Current Protocols in Immunology, Volumes 1 and 2, Coligen et al., Ed. Wiley-Interscience, New York, N.Y., Pubs. (1991) for example and radioactivity can be measured using scintillation counting. (b) Colloidal gold particles. (c) Fluorescent labels including, but are not limited to, rare earth chelates (europium chelates), Texas Red, rhodamine, fluorescein, dansyl, Lissamine, umbelliferone, phycocrytherin, phycocyanin, or commercially available fluorophores such SPECTRUM ORANGE7 and SPECTRUM GREEN7 and/or derivatives of any one or more of the above. The fluorescent labels can be conjugated to the antibody using the techniques disclosed in Current Protocols in Immunology, supra, for example. fluorescence can be quantified using a fluorimeter. (d) Various enzyme-substrate labels are available and U.S. Pat. No. 4,275,149 provides a review of some of these. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, .beta.-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed. J. Langone & H. Van Vunakis), Academic press, New York, 73:147-166 (1981).

Examples of enzyme-substrate combinations include, for example: (i) Horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB)); (ii) alkaline phosphatase (AP) with para-Nitrophenyl phosphate as chromogenic substrate; and (iii) .beta.-D-galactosidase (.beta.-D-Gal) with a chromogenic substrate (e.g., p-nitrophenyl-.beta.-D-galactosidase) or fluorogenic substrate (e.g., 4-methylumbelliferyl-.beta.-D-galactosidase).

Numerous other enzyme-substrate combinations are available to those skilled in the art. For a general review of these, see U.S. Pat. Nos. 4,275,149 and 4,318,980. Sometimes, the label is indirectly conjugated with the antibody. The skilled artisan will be aware of various techniques for achieving this. For example, the antibody can be conjugated with biotin and any of the four broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten and one of the different types of labels mentioned above is conjugated with an anti-hapten antibody. Thus, indirect conjugation of the label with the antibody can be achieved.

Aside from the sample preparation procedures discussed above, further treatment of the tissue section prior to, during or following IHC may be desired. For example, epitope retrieval methods, such as heating the tissue sample in citrate buffer may be carried out (see, e.g., Leong et al. Appl. Immunohistochem. 4(3):201 (1996)).

Following an optional blocking step, the tissue section is exposed to primary antibody for a sufficient period of time and under suitable conditions such that the primary antibody binds to the target protein antigen in the tissue sample. Appropriate conditions for achieving this can be determined by routine experimentation. The extent of binding of antibody to the sample is determined by using any one of the detectable labels discussed above. In some embodiments, the label is an enzymatic label (e.g. HRPO) which catalyzes a chemical alteration of the chromogenic substrate such as 3,3′-diaminobenzidine chromogen. In one embodiment, the enzymatic label is conjugated to antibody which binds specifically to the primary antibody (e.g. the primary antibody is rabbit polyclonal antibody and secondary antibody is goat anti-rabbit antibody).

Specimens thus prepared may be mounted and coverslipped. Slide evaluation is then determined, e.g., using a microscope, and staining intensity criteria, routinely used in the art, may be employed.

In some embodiments, when IHC is used, a tiered system of staining is used to determine whether a cell or collection of cells overexpresses a protein. For example, in some embodiments, a four-tiered system is used in which the tiers are no staining (0), 1+, 2+, and 3+, where 1+, 2+, and 3+ indicate increasing levels of staining, respectively. In some such embodiments, greater than 1+, greater than 2+, or greater than 3+ may be used to indicate protein overexpression. As a nonlimiting example, if a particular cell type typically shows no staining for a protein in an IHC assay, then any staining in that IHC assay (i.e., 1+, 2+, or 3+) may be indicative as protein overexpression. As a further nonlimiting example, if a particular cell type typically shows little to no staining for the protein in an IHC assay, then any staining above 1+ in that IHC assay (i.e., 2+ or 3+) may be indicative as protein overexpression. One skilled in the art can determine the staining level that indicates protein overexpression depending on the particular IHC assay (including the particular antibody), the cell type, etc.

In some embodiments, a breast cancer is characterized as HER2 positive or HER2 negative according to IHC. In some such embodiments, a breast cancer is characterized as HER2 negative when the IHC cell membrane stain intensity is 0 or 1+. In some embodiments, a breast cancer is characterized as HER2 positive when the IHC cell membrane stain intensity is 3+. In some embodiments, the HER2 status of a breast cancer is equivocal when the IHC cell membrane stain intensity is 2+. In some embodiments of an equivocal HER2 status by IHC, a HER2 FISH assay is used to determine whether the HER2 gene is amplified. In some such embodiments, if the HER2 gene is amplified, the breast cancer is considered to be HER2 positive.

Nonlimiting exemplary methods of determining whether a cancer comprises HER2 overexpression and/or amplification (i.e., whether the cancer is “HER2 positive”) are described, e.g., in WO99/31140; US2003/0170234A1; US2003/0147884; WO01/89566; US2002/0064785; US2003/0134344; U.S. Pat. No. 6,573,043; U.S. Pat. No. 6,905,830; and US2003/0152987.

In some embodiments, the status of the estrogen receptor (ER) and/or progesterone receptor (PR) is determined according to the American Society of Clinical Oncology/College of American Pathologists Guideline Recommendations for Immunohistochemical Testing of Estrogen and Progesterone Receptors in Breast Cancer, J. Clin. Oncol., 2010, 28: 2784-2795 (“Guidelines”), which is incorporated by reference herein in its entirety for any purpose. The recommendations indicate that a breast cancer should be considered ER positive or PR positive when ≧1% of the tumor cell nuclei are immunoreactive in the corresponding IHC assay, and should be considered ER negative or PR negative when <1% of tumor cell nuclei are immunoreactive in the corresponding IHC assay.

Any suitable method of determining mRNA overexpression may be used. Methods for the evaluation of mRNAs in cells are well known and include, for example, hybridization assays using complementary DNA probes (such as in situ hybridization using labeled riboprobes specific for a target mRNA, Northern blots, and related techniques) and various nucleic acid amplification assays (such as RT-PCR using complementary primers specific for a target mRNA (or a cDNA reverse-transcribed from the target mRNA) and other amplification type detection methods, such as, for example, branched DNA, SISBA, TMA and the like).

Tissue or cell samples from mammals can be conveniently assayed for mRNAs using Northern, dot blot or PCR analysis. For example, RT-PCR assays such as quantitative PCR assays are well known in the art. In some embodiments, mRNA expression levels are levels quantified using real-time qRT-PCR. In some embodiments of the invention, a method for detecting a target mRNA in a biological sample comprises producing cDNA from the sample by reverse transcription using at least one primer; amplifying the cDNA so produced using a target polynucleotide as sense and antisense primers to amplify target cDNAs therein; and detecting the presence of the amplified target cDNA. In addition, such methods can include one or more steps that allow one to determine the levels of target mRNA in a biological sample (e.g., by simultaneously examining the levels a comparative control mRNA sequence of a “housekeeping” gene such as an actin family member). Optionally, the sequence of the amplified target cDNA can be determined.

Optional methods of the invention include protocols which examine or detect mRNAs, such as target mRNAs, in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. Hybridization of a labeled probe with a particular array member indicates that the sample from which the probe was derived expresses that gene. Differential gene expression analysis of disease tissue can provide valuable information. Microarray technology utilizes nucleic acid hybridization techniques and computing technology to evaluate the mRNA expression profile of thousands of genes within a single experiment. (see e.g., WO 01/75166 published Oct. 11, 2001; (see, for example, U.S. Pat. No. 5,700,637, U.S. Pat. No. 5,445,934, and U.S. Pat. No. 5,807,522, Lockart, Nature Biotechnology, 14:1675-1680 (1996); Cheung, V. G. et al., Nature Genetics 21(Suppl):15-19 (1999) for a discussion of array fabrication). DNA microarrays are miniature arrays containing gene fragments that are either synthesized directly onto or spotted onto glass or other substrates. Thousands of genes are usually represented in a single array. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. Currently two main types of DNA microarrays are being used: oligonucleotide (usually 25 to 70 mers) arrays and gene expression arrays containing PCR products prepared from cDNAs. In forming an array, oligonucleotides can be either prefabricated and spotted to the surface or directly synthesized on to the surface (in situ). In some embodiments, a DNA microarray is a single-nucleotide polymorphism (SNP) microarrays, e.g., Affymetrix® SNP Array 6.0.

The Affymetrix GeneChip® system is a commercially available microarray system which comprises arrays fabricated by direct synthesis of oligonucleotides on a glass surface. Probe/Gene Arrays: Oligonucleotides, usually 25 mers, are directly synthesized onto a glass wafer by a combination of semiconductor-based photolithography and solid phase chemical synthesis technologies. Each array contains up to 400,000 different oligos and each oligo is present in millions of copies. Since oligonucleotide probes are synthesized in known locations on the array, the hybridization patterns and signal intensities can be interpreted in terms of gene identity and relative expression levels by the Affymetrix Microarray Suite software. Each gene is represented on the array by a series of different oligonucleotide probes. Each probe pair consists of a perfect match oligonucleotide and a mismatch oligonucleotide. The perfect match probe has a sequence exactly complimentary to the particular gene and thus measures the expression of the gene. The mismatch probe differs from the perfect match probe by a single base substitution at the center base position, disturbing the binding of the target gene transcript. This helps to determine the background and nonspecific hybridization that contributes to the signal measured for the perfect match oligo. The Microarray Suite software subtracts the hybridization intensities of the mismatch probes from those of the perfect match probes to determine the absolute or specific intensity value for each probe set. Probes are chosen based on current information from Genbank and other nucleotide repositories. The sequences are believed to recognize unique regions of the 3′ end of the gene. A GeneChip Hybridization Oven (“rotisserie” oven) is used to carry out the hybridization of up to 64 arrays at one time. The fluidics station performs washing and staining of the probe arrays. It is completely automated and contains four modules, with each module holding one probe array. Each module is controlled independently through Microarray Suite software using preprogrammed fluidics protocols. The scanner is a confocal laser fluorescence scanner which measures fluorescence intensity emitted by the labeled cRNA bound to the probe arrays. The computer workstation with Microarray Suite software controls the fluidics station and the scanner. Microarray Suite software can control up to eight fluidics stations using preprogrammed hybridization, wash, and stain protocols for the probe array. The software also acquires and converts hybridization intensity data into a presence/absence call for each gene using appropriate algorithms. Finally, the software detects changes in gene expression between experiments by comparison analysis and formats the output into .txt files, which can be used with other software programs for further data analysis.

### EXAMPLES

The examples discussed below are intended to be purely exemplary of the invention and should not be considered to limit the invention in any way. The examples are not intended to represent that the experiments below are all or the only experiments performed. It is understood that various other embodiments may be practiced, given the general description provided above.

Efforts have been made to ensure accuracy with respect to numbers used (for example, amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

In Examples 1 to 6, the dosages of FGFR1-ECD.339-Fc are calculated using EC=1.42 mL/mg*cm. See Table 1.

### Example 1

### Certain Lung Cancer Xenograft Models with FGFR1 Gene Amplification were More Sensitive to FGFR1-ECD.339-Fc-Mediated Growth Inhibition than Certain Non-FGFR1 Gene Amplified Lung Cancer Xenograft Models

The impact of FGFR1-ECD.339-Fc on tumor growth was compared between FGFR1 gene amplified and non-amplified lung cancer xenograft models. Lung cancer cell lines with FGFR1-amplification examined in this experiment were as follows: DMS53 (SCLC, 5 copies FGFR1 gene per cell), DMS114 (SCLC, 10 copies FGFR1 gene per cell), NCI-H1518 (NSCLC, 6 copies FGFR1 gene per cell), and NCI-H520 (NSCLC, 8 copies FGFR1 gene per cell). Lung cancer cell lines without FGFR1-amplification examined in this experiment were as follows: A549, NCI-H460, NCI-H226, NCI-H2126, NCI-H441, NCI-H358, NCI-H522 and Colo699. Non-amplified cell lines were purchased from ATTC (Manassas, Va.) and cultured according to supplier instructions. Lung cancer xenograft models using non-FGFR1 gene amplified cell lines were carried out as follows. Six week old female SCID mice were purchased from Charles River Laboratories (Wilmington, Mass.) and were acclimated for 1 week before the start of the study. Lung cancer cell lines were cultured until they reached 85-90% confluence. Cells were harvested and resuspended in cold Ca²⁺ and Mg²⁺ free phosphate buffered saline (PBS) containing 50% Matrigel at 5×10⁷ cells per milliliter. The cells were implanted subcutaneously over the right flank of the mice at 5×10⁶ cells/100 μl/mouse. One day following cell implantation mice were sorted and randomized (n=10) and treatment initiated as described below.

A panel of patient-derived xenograft (PDX) models of lung cancer without FGFR1-amplification was also examined for sensitivity to FGFR1-ECD.339-Fc. PDX xenografts have been transplanted directly from cancer patients into nude mice without in vitro tissue culture. The tumor xenografts retain most of the characteristics of the parental patient tumors including histology and sensitivity to anticancer drugs. Lung PDX models examined were as follows: PDX D35087, PDX D37638, PDX D35376, LXFL-430, LXFE-937, LXFE-397, LXFA-737 and LXFA-629. Preliminary pathology and patient characteristics for the lung PDXs examined are outlined in Table 2.

TABLE 2
Characteristics of lung cancer patient-derived xenograph (PDX) models

| Tumor | Tissue | | Differen- | Patient | | |
|---|---|---|---|---|---|---|
| No. | type | Origin | tiation | age | Gender | Stage |
| LXFE_ | Squa- | Lung | moder- | 37 | female | T3N1M0 |
| 937 | mous | | ately | | | |
| | | | differen- | | | |
| | | | tiated | | | |
| LXFE_ | Squa- | Lung | poorly | 56 | male | T1N0Mx |
| 397 | mous | | differen- | | | |
| | | | tiated | | | |
| LXFL_ | Large | Lung | poorly | 53 | male | T2N1M0 |
| 430 | cell | | differen- | | | |
| | | | tiated | | | |
| LXFA_ | Adeno | Lung | poorly | 59 | male | T3N2Mx |
| 629 | | | differen- | | | |
| | | | tiated | | | |
| LXFA_ | Adeno | Lung | moder- | 56 | male | T3N2Mx |
| 737 | | | ately | | | |
| | | | differen- | | | |
| | | | tiated | | | |
| PDX | Squa- | Lung | moder- | — | — | T3N0M0 |
| D35087 | mous | | ately | | | |
| | | | differen- | | | |
| | | | tiated | | | |
| PDX | Squa- | Lung | poorly | — | — | T3N2M0 |
| D37638 | mous | | differen- | | | |
| | | | tiated | | | |
| PDX | Squa- | Lung | moder- | — | — | T2N0M0 |
| D35376 | mous | | ately | | | |
| | | | differen- | | | |
| | | | tiated | | | |

Six week old female SCID mice were purchased from Charles River Laboratories (Wilmington, Mass.) and were acclimated for 1 week before the start of the study. PDX tumor fragments were obtained from xenografts in serial passage in donor SCID mice. After removal of tumors from donor mice, they were cut into fragments (1-2 mm diameter, ˜25 mgs) and placed in RPMI 1640 culture medium until subcutaneous implantation. Recipient mice were anaesthetized by inhalation of isoflurane. A small pocket was formed with blunt forceps and one chunk of tumor PDX was placed in the pocket. The wound was sealed using dermabond glue and a drop of bupivicaine placed on the incision. One day following PDX implantation mice were sorted and randomized (n=10) and treatment initiated as described below.

FGFR1-ECD.339-Fc was formulated in PBS at 3 mg/ml and administered intraperitoneally (i.p.) at 15 mg/kg (300 μg/100 μl/mouse) twice a week for four to eight weeks depending on the growth rate of the PDX tumor implanted. Human albumin was purchased from Grifols USA (Los Angeles, Calif.; Cat. No. NDC 61953-0002-1), diluted to a working stock (3 mg/ml) with 0.9% sodium chloride, and was used as negative control at 300 μg/100 μl/mouse (15 mg/kg) administered twice a week for four to eight weeks depending on the growth rate of the PDX tumor implanted.

Tumor sizes were measured in each mouse on days 11, 18, 25, 32, 39 and 46 following the day of tumor cell inoculation. The length and width of each tumor was measured using calipers and the tumor size calculated according to the formula:

Tumor size (mm³)=(width (mm)×length (mm))²/2

Mice were euthanized as a “cancer death” when the subcutaneous tumor volumes exceeded 2000 mm³ or when the tumors became excessively necrotic.

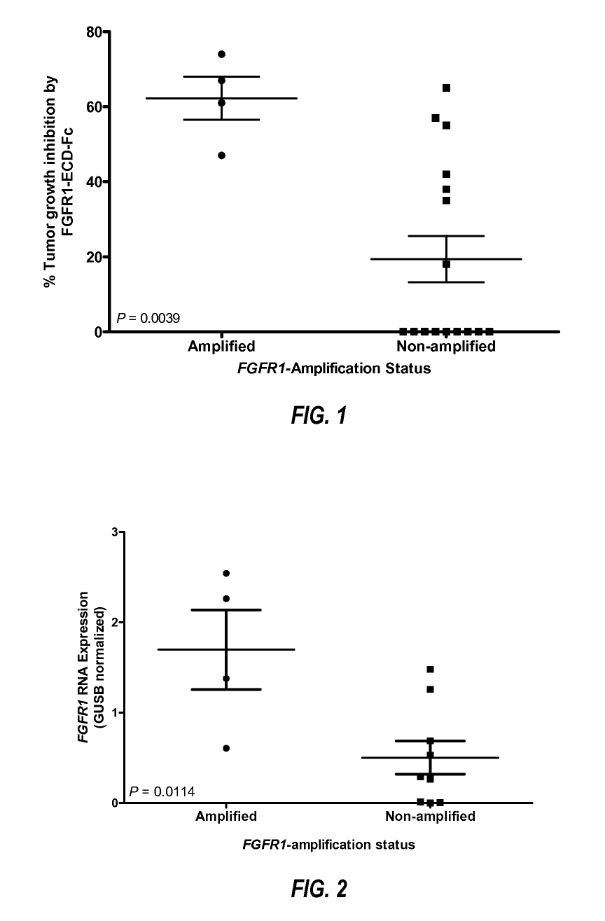

Percentage tumor growth inhibition by FGFR1-ECD.339-Fc was determined by area-under-the-curve (AUC) analysis of xenograft growth curves treated with FGFR1-ECD.339-Fc compared to albumin control. FIG. 1 shows a scatterplot of the results of this analysis. Lung cancer xenografts with FGFR1 gene amplification had an average a 56% reduction in tumor growth with FGFR1-ECD.339-Fc treatment. In comparison, lung cancer xenografts without FGFR1 gene amplification displayed an average 22% decrease in xenograft growth with FGFR1-ECD.339-Fc treatment compared to control. The difference in FGFR1-ECD.339-Fc-mediated xenograft inhibition between FGFR1 gene amplified and non-amplified lung cancer xenograft models was statistically significant (P=0.0333).

Thus, FGFR1 gene amplified tumor cells were found to be more sensitive to FGFR1-ECD.339-Fc administration than tumor cells with a non-amplified FGFR1 gene.

### Example 2

### FGFR1 Overexpression in FGFR1 Gene-Amplified and Non-Amplified Lung Cancer Cell Lines and Xenografts

The expression of the FGFR1 at the RNA level was compared between FGFR1 gene amplified and non-amplified lung cancer cell lines, xenograft models, and PDX models. Lung cancer cell lines with FGFR1-amplification examined in this experiment were as follows: DMS53 (SCLC, 5 copies FGFR1 gene per cell), DMS114 (SCLC, 10 copies FGFR1 gene per cell), NCI-H1518 (NSCLC, 6 copies FGFR1 gene per cell), and NCI-H520 (NSCLC, 8 copies FGFR1 gene per cell). Lung cancer cell lines without FGFR1 gene amplification examined in this experiment were as follows: A549, NCI-H460, NCI-H226, NCI-H2126, NCI-H441, NCI-H358, NCI-H522, MSTO-211H, and Colo699. Non-amplified cell lines were purchased from ATTC (Manassas, Va.) and cultured according to supplier instructions. A panel of patient-derived xenograft (PDX) models of lung cancer without FGFR1 gene amplification was also examined for FGFR1 mRNA expression. Lung PDX models examined were as follows: PDX D35087, PDX D37638, PDX D35376, LXFL-430, LXFE-937, LXFE-397, LXFA-737, and LXFA-629. Preliminary pathology and patient characteristics for the lung PDXs examined are outlined above in Table 2.

RNA was extracted from cell lines grown in vitro or tumor xenografts grown in vivo using the RNAeasy® mini kit (cat. No. 74104, Qiagen, Germany). Extracted RNA was treated with DNAse I prior to creating cDNA with random hexamer priming and reverse transcriptase using the QuantiTect Reverse Transcription Kit (cat. No. 205311, Qiagen, Germany). Human FGFR1 RNA expression was determined using an FGFR1 QuantiTect Primer Assay (Hs_FGFR1_{—}1_SG, cat. No. QT00102837, Qiagen, Germany) and a human GUSB control reference QuantiTect Primer Assay (Hs_GUSB_{—}1_SG, cat. No. QT00046046, Qiagen, Germany). QuantiTect SYBR Green PCR Kits (cat. No. 204145, Qiagen, Germany) were used to quantify mRNA expression levels using real-time qRT-PCR and an ABI Prism ViiA™ 7 Real-Time PCR System (Applied Biosystems, Foster City, Calif.). Relative gene expression quantification was calculated according to the comparative Ct method using human GUSB as a reference and commercial RNA controls (Stratagene, La Jolla, Calif.). Relative quantification was determined according to the formula: 2^{-ΔCt sample-ΔCt calibrator)}.

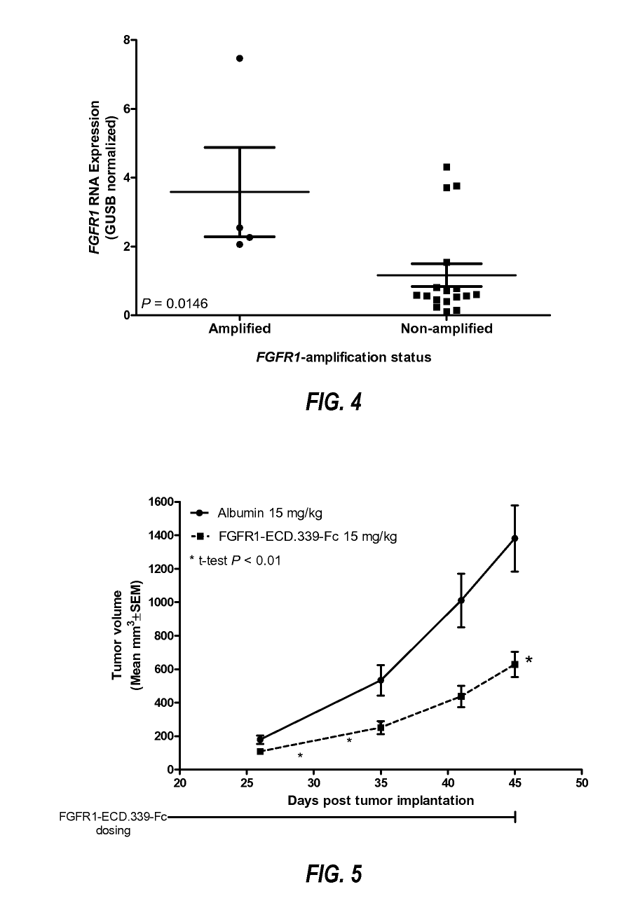

GUSB-normalized FGFR1 RNA expression was compared between lung cancer cell lines (FIG. 2) and xenograft models (FIG. 4) with and without FGFR1 gene amplification.

FIG. 2 shows a scatterplot of FGFR1 RNA expression in cell lines with and without FGFR1 gene amplification. Lung cancer cell lines with FGFR1 gene amplification have a statistically significant increase (P=0.0114) in FGFR1 mRNA expression compared to cell lines without FGFR1 gene amplification. FIG. 2 also demonstrates that a sub-population of lung cancer cell lines have high FGFR1 mRNA expression in the absence of FGFR1 gene amplification. NCI-H226, which has a GUSB normalized gene expression of FGFR1 of 1.48, and NCI-H522, which has a GUSB normalized gene expression of FGFR1 of 1.26, represent the two uppermost outlier points in the non-amplified lung cancer cell line population.

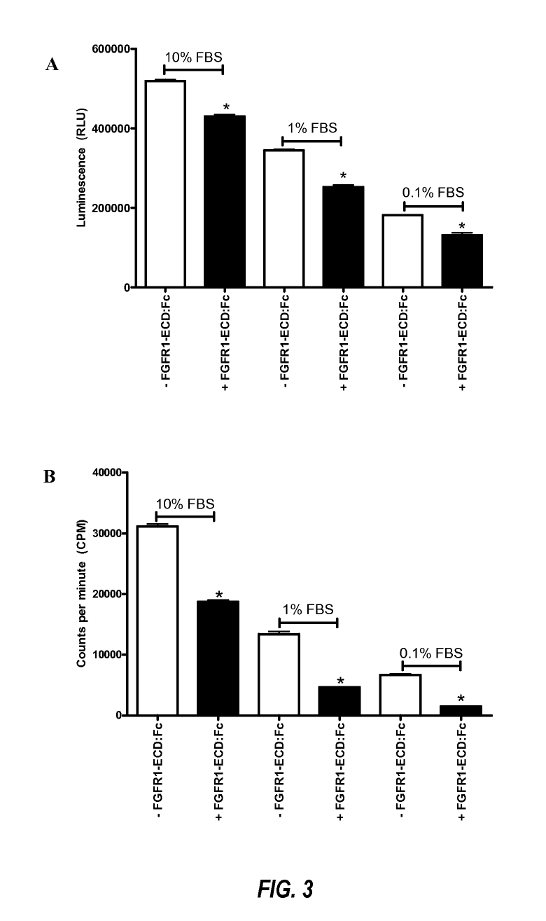

NCI-H226 and NCI-H522 were also sensitive to FGFR1-ECD.339-Fc in vitro, having decreased cell proliferation and number using the tritiated thymidine ([3H]-TdR) incorporation assay and CellTiter-Glo® Luminescent Cell Viability Assay (Promega, Madison, Wis.), respectively. FIG. 3A shows results from the CellTiter-Glo® assay for the NCI-H226 cell line, demonstrating that cell number was significantly (* indicates P=>0.05) reduced by FGFR1-ECD.339-Fc incubation in the NCI-H226 cell line, which does not have FGFR1-amplification. P-values were determined using an unpaired t-test. See, e.g., Mathematical Statistics and Data Analysis, 1988, Wadsworth & Brooks, Pacific Grove, Calif.

FIG. 3B shows results from the tritiated thymidine incorporation assay for the NCI-H226 cell line, demonstrating that cell proliferation was significantly (* indicates P=>0.05) reduced by FGFR1-ECD.339-Fc incubation in the NCI-H226 cell line, which does not have FGFR1 gene amplification. P-values were determined using an unpaired t-test. The control ECD Fc had little no impact on NCI-H226 cell proliferation.

Thus, certain lung cancer cell lines that do not have FGFR1 gene amplification, but which have FGFR1 overexpression, are sensitive to FGFR1-ECD.339-Fc treatment.

FIG. 4 shows a scatterplot of FGFR1 mRNA expression comparing FGFR1 gene amplified to non-amplified lung cancer xenografts. Xenograft models with FGFR1 gene amplification had a statistically significant (P=0.0146) increase in FGFR1 RNA levels compared to non-amplified cell lines. In addition, in agreement with the in vitro data, a sub-population of lung cancer xenograft models has high FGFR1 RNA expression in the absence of FGFR1 gene amplification. Xenograft models NCI-H226, NCI-H522 and PDX D35087 represent the 3 outlier points for FGFR1 RNA expression in the non-amplified lung models (FIG. 4), with GUSB-normalized gene expression levels of 3.70, 3.75 and 4.30, respectively.

NCI-H226, NCI-H522, and PDX D35087 were also sensitive to FGFR1-ECD.339-Fc in vivo, demonstrating a statistically significant (P<0.05) reduction in tumor growth of 55, 42 and 57% respectively with FGFR1-ECD.339-Fc treatment. For PDX D35087, the experiment was carried out substantially as described in Example 1.

Tumor sizes were measured in each mouse on days 26, 35, 41 and 45 following the day of PDX D35087 implantation. The length and width of each tumor was measured using calipers and the tumor size calculated according to the formula:

Tumor size (mm³)=(width (mm)×length (mm))²/2

FIG. 5 shows the results of this experiment. Mice that received FGFR1-ECD.339-Fc showed an inhibition of tumor growth compared to albumin-treated animals. Comparison of PDX 35087 tumor volume at day 45 in the FGFR1-ECD.339-Fc treatment group and vehicle treated group indicated that this result was statistically significant (P<0.01). P-values were calculated using an ANOVA analysis. See, e.g., Mathematical Statistics and Data Analysis, 1988, Wadsworth & Brooks, Pacific Grove, Calif. This analysis demonstrated that FGFR1-ECD.339-Fc significantly reduced tumor growth in the PDX lung tumor model D35087, which does not have amplification of the FGFR1 gene, but expresses relatively high-levels of FGFR1 mRNA.

Thus, certain lung cancer xenograft models that do not have FGFR1 gene amplification, but which have FGFR1 overexpression, are sensitive to FGFR1-ECD.339-Fc treatment.

### Example 3

### Predictors of FGFR1-ECD.339-Fc Response

The RNA expression of a panel of FGFR1-related genes including FGF ligands, FGF receptors, FGF binding proteins, FGF signaling molecules, and a group of angiogenesis-related targets was determined in a set of 35 tumor cell lines and xenografts using qRT-PCR. RNA was extracted from cell lines grown in vitro or tumor xenografts grown in vivo using the RNAeasy® mini kit (Qiagen, Germany). Extracted RNA was treated with DNAse I prior to creating cDNA with random hexamer priming and reverse transcriptase using the QuantiTect Reverse Transcription Kit (Qiagen, Germany). Human and mouse RNA expression was determined using QuantiTect Primer Assays (Qiagen, Germany) employing a human GUSB control reference QuantiTect Primer Assay (Qiagen, Germany). QuantiTect SYBR Green PCR Kits (Qiagen, Germany) were used to quantify mRNA expression levels using real-time qRT-PCR and an ABI Prism ViiA™ 7 Real-Time PCR System (Applied Biosystems, Foster City, Calif.). Relative gene expression quantification was calculated according to the comparative Ct method using human GUSB as a reference and commercial RNA controls (Stratagene, La Jolla, Calif.). Relative quantification was determined according to the formula: 2^{-(ΔCt sample-ΔCt calibrator)}.

The tumor cell lines and xenografts used in this experiment are shown in Table 3. Also shown in Table 3 are the dosing schedule for FGFR1-ECD.339-Fc in a mouse xenograft model, the percent tumor growth inhibition (TGI (%)) and the statistical significance of the tumor growth inhibition (P Value), as well as whether the FGFR1 gene is amplified in the cell line.

TABLE 3
Anti-tumor activity of FGFR1-ECD.339-Fc in a panel of xenograft models

| | | | | | | | | | FGFR1 |
|---|---|---|---|---|---|---|---|---|---|
| Tumor | Xenograft | Cell line/ | Dosing | | | Dose | TGI | | amp. |
| type | model | PDX | route | Dose | | sched. | (%) | P Value | status |
| Colon | HCT116 | Cell Line | IP | 15 | mg/kg | BIW | 0% | ns | Non- |
| | | | | | | | | | amplified |
| | Colo205 | Cell Line | IV | 5 | mg/kg | BIW | 38% | P < 0.001 | Non- |
| | | | | | | | | | amplified |
| | Colo201 | Cell Line | IP | 15 | mg/kg | BIW | 0% | ns | Non- |
| | | | | | | | | | amplified |
| Renal | G-401 | Cell Line | IP | 15 | mg/kg | BIW | 36% | P < 0.05 | Non- |
| | | | | | | | | | amplified |
| | A498 | Cell Line | IP | 15 | mg/kg | BIW | 7% | ns | Non- |
| | | | | | | | | | amplified |
| | Caki-1 | Cell Line | IV | 10 | mg/kg | BIW | 81% | P < 0.001 | Non- |
| | | | | | | | | | amplified |
| Lung | A549 | Cell Line | IP | 10 | mg/kg | BIW | 38% | P < 0.05 | Non- |
| | | | | | | | | | amplified |
| | NCI-H460 | Cell Line | IP | 10 | mg/kg | BIW | 35% | P < 0.05 | Non- |
| | | | | | | | | | amplified |
| | NCI-H226 | Cell Line | IP | 15 | mg/kg | 3x/w | 55% | P < 0.001 | Non- |
| | | | | | | | | | amplified |
| | NCI-H520 | Cell Line | IP | 20 | mg/kg | BIW | 47% | P < 0.05 | Amplified |
| | NCI-H1703 | Cell Line | IP | 15 | mg/kg | BIW | 31% | P < 0.05 | Amplified |
| | NCI-H2126 | Cell Line | IP | 15 | mg/kg | BIW | 0% | ns | Non- |
| | | | | | | | | | amplified |
| | NCI-H441 | Cell Line | IP | 15 | mg/kg | BIW | 0% | ns | Non- |
| | | | | | | | | | amplified |
| | NCI-H358 | Cell Line | IP | 15 | mg/kg | BIW | 0% | ns | Non- |
| | | | | | | | | | amplified |
| | NCI-H522 | Cell Line | IP | 10 | mg/kg | BIW | 42% | P < 0.05 | Non- |
| | | | | | | | | | amplified |
| | NCI-H1581 | Cell Line | IP | 15 | mg/kg | BIW | 74% | P = 0.002 | Amplified |
| | DMS53 | Cell Line | IP | 15 | mg/kg | BIW | 64% | 0.003 | Amplified |
| | DMS114 | Cell Line | IP | 15 | mg/kg | BIW | 64% | P < 0.001 | Amplified |
| | Calu-1 | Cell Line | IP | 15 | mg/kg | BIW | 0% | ns | Non- |
| | | | | | | | | | amplified |
| | D35087 | PDX | IP | 15 | mg/kg | BIW | 57% | P < 0.01 | Non- |
| | | | | | | | | | amplified |
| | D37638 | PDX | IP | 15 | mg/kg | BIW | 20% | ns | Non- |
| | | | | | | | | | amplified |
| | D35376 | PDX | IP | 15 | mg/kg | BIW | 15% | ns | Non- |
| | | | | | | | | | amplified |
| | LXFA-737 | PDX | IP | 15 | mg/kg | BIW | 0%** | ns | Non- |
| | | | | | | | | | amplified |
| | LXFA-629 | PDX | IP | 15 | mg/kg | BIW | 65% | P = 0.007 | Non- |
| | | | | | | | | | amplified |
| Mesothelioma | MSTO-211H | Cell Line | IP | 15 | mg/kg | BIW | 64% | P < 0.0001 | Non- |
| | | | | | | | | | amplified |
| Glioblastoma | U-87 | Cell Line | IP | 15 | mg/kg | BIW | 0% | ns | Non- |
| | | | | | | | | | amplified |
| | U-118 | Cell Line | IP | 15 | mg/kg | BIW | 36% | ns | Non- |
| | | | | | | | | | amplified |
| | U-251 | Cell Line | IP | 15 | mg/kg | BIW | 48% | P = 0.0078 | Non- |
| | | | | | | | | | amplified |
| Retino- | Y79 | Cell Line | IP | 10 | mg/kg | BIW | 21% | ns | Non- |
| blastoma | | | | | | | | | amplified |
| Prostate | Du145 | Cell Line | IP | 0.15 | mg/kg | 3x/w | 31% | ns | Non- |
| | | | | | | | | | amplified |
| Endometrial | MFE-280 | Cell Line | IP | 15 | mg/kg | BIW | 96% | P < 0.001 | Non- |
| | | | | | | | | | amplified |
| | HEC-1B | Cell Line | IP | 15 | mg/kg | BIW | 30% | P < 0.05 | Non- |
| | | | | | | | | | amplified |
| | MFE-319 | Cell Line | IP | 15 | mg/kg | BIW | 22% | ns | Non- |
| | | | | | | | | | amplified |
| Breast | MDA-MB-231 | Cell Line | IP | 15 | mg/kg | BIW | 0% | ns | Non- |
| | | | | | | | | | amplified |
| | JIMT1 | Cell Line | IP | 1 | mg/kg | BIW | 28% | P < 0.05 | Non- |
| | | | | | | | | | amplified |

**% TGI for LXFA-737 was less than 0.

An exemplary xenograft experiment is as follows. For Caki-1 and MSTO-211H, five million cells were implanted subcutaneously over the right flank of SCID mice (N=10 per group). FGFR1-ECD.339-Fc or albumin was administered i.p. twice a week at the dose indicated in Table 3. FIG. 8 shows anti-tumor activity of FGFR1-ECD.339-Fc in selected xenograft models. Representative tumor growth curves are shown for a renal cancer, Caki-1, (A), and mesothelioma, MSTO-211H, (B) xenograft cancer model. In the renal cell carcinoma (RCC) Caki-1 model, administration of FGFR1-ECD.339-Fc at 10 mg/kg twice a week for 6 weeks resulted in 81% (P<0.001) tumor growth inhibition (TGI; FIG. 8A). In the MSTO-211H mesothelioma model, FGFR1-ECD.339-Fc administration reduced tumor growth (FIG. 8B) by 64% (P<0.0001). In responding tumors, FGFR1-ECD.339-Fc significantly reduced tumor volume as assessed by area-under-the-curve (AUC) analysis. Responses were observed in 19/35 (54%) of the models examined, with a range of 25-96% inhibition (see Table 3).

In order to further understand the potential molecular determinants that make xenograft models sensitive to treatment with FGFR1-ECD.339-Fc, the RNA expression of a panel of genes including FGF ligands, FGF receptors, FGF binding proteins and FGF signaling molecules was examined using qRT-PCR in certain xenograft models from Table 3.

Gene expression was then correlated to FGFR1-ECD.339-Fc response to determine RNA expression signatures positively and negatively correlated with anti-tumor activity. Table 4 shows the results of that analysis. In addition to FGF2, RNA expression of FGF18 (P=0.02227) was also positively (6.9-fold) correlated with FGFR1-ECD.339-Fc anti-tumor activity. The downstream target gene of FGF signaling, ets variant 4 (ETV4), was the most significant (P=0.01639) gene for its positive (2.897-fold) association with FGFR1-ECD.339-Fc activity. Expression of FGFR1 (P=0.01276), including the FGFR1IIIk splice variant (P=0.01603), was a positive predictor for FGFR1-ECD.339-Fc response. Expression of the FGFR1IIIb splice variant was not correlated with FGFR1-ECD.339-Fc response in that experiment. In addition to FGFR1, expression of the FGFR3IIIc receptor (P=0.02488) was also positively correlated with FGFR1-ECD.339-Fc response, reflecting the potential overlap in FGF-ligand binding affinities between the Inc-splice isoforms of FGFR1 and FGFR3 receptors. Significant genes with a negative association with FGFR1-ECD.339-Fc activity were not found in this analysis.

TABLE 4
Statistical analysis of FGF-related gene expression in relation to
FGFR1-ECD.339-Fc anti-tumor response in xenograft models

| Gene | Ratio^{§} | P value^{†} | Gene | Ratio^{§} | P value^{†} |
|---|---|---|---|---|---|
| ETV4 | 2.897 | 0.01639 | SPRY3 | 1.665 | 0.4944 |
| FGFR1 | 2.447 | 0.01669 | SPRY1 | 1.394 | 0.5008 |
| FGFR3IIIc | 9.863 | 0.01944 | DUSP6 | 0.6418 | 0.507 |
| FGF18 | 6.915 | 0.02227 | FGF19 | 1.203 | 0.5338 |
| FGF2 | 247.7 | 0.03569 | FLRT1 | 1.158 | 0.5676 |
| FGFR1IIIc | 3.647 | 0.0431 | FGF3 | 1.431 | 0.5699 |
| DUSP4 | 0.09578 | 0.08166 | FGFR4 | 1.347 | 0.5755 |
| TNC | 0.0345 | 0.1212 | FGF9 | 0.5356 | 0.6102 |
| VIM | 5.155 | 0.1448 | FGFR3 | 1.767 | 0.6165 |
| ETV5 | 1.447 | 0.1567 | SPRY2 | 0.3142 | 0.6313 |
| FGFBP3 | 1.84 | 0.1592 | SERPINE1 | 0.333 | 0.6642 |
| PLAU | 0.3842 | 0.1781 | FGF21 | 1.935 | 0.6744 |
| PLAUR | 0.3805 | 0.2408 | FLRT2 | 0.2276 | 0.693 |
| FGF7 | 1.991 | 0.243 | FGFR2b | 0.9266 | 0.7897 |
| FGF5 | 24.79 | 0.2691 | FGF6 | 0 | 0.8316 |
| KDR | 0.5892 | 0.2742 | FGFBP1 | 0.5 | 0.8372 |
| FGF11 | 2.153 | 0.2944 | SOX9 | 1.181 | 0.8372 |
| MET | 0.4225 | 0.2962 | SPRY4 | 0.9028 | 0.8372 |
| FGF2 | 5.48 | 0.3015 | NCAM1 | 1.661 | 0.8731 |
| DUSP5 | 0.4765 | 0.3238 | FGF8 | 1.052 | 0.9552 |
| FGF22 | 1.604 | 0.3484 | ELK4 | 1.062 | 0.9815 |
| FGF10 | 1.91 | 0.3518 | CDH1 | 0.1158 | 0.9818 |
| FGFR2 | 1.402 | 0.3587 | ELK3 | 1.157 | 0.9818 |
| FGF1 | 0.09845 | 0.398 | FGFBP2 | 0.7737 | 0.9818 |
| FGFR2IIIc | 5.546 | 0.4195 | FGF16 | 1.076 | 1 |
| FGF17 | 1.334 | 0.4361 | FLRT3 | 0.7523 | 1 |
| FGFR3IIIb | 1.08 | 0.451 | | | |
| FGF20 | 5.967 | 0.4729 | | | |
| FGFR1IIIb | 0.6493 | 0.486 | | | |

^{§}Gene expression ratio determined by median gene expression in FGFR1-ECD.339-Fc responders/non-responders
^{†}P-values are determined by a Mann-Whitney test of PCR gene expression in responders vs. non-responders for each gene using all models in Table 3.

To determine what RNA factors may determine lung xenograft response in the absence of FGFR1-gene amplification, the correlation of FGFR1-ECD.339-Fc response in the non-FGFR1 amplified subset of lung models was examined (N=13). The results of that analysis are shown in Table 5. FGF2 expression was up-regulated >3,000 fold in responding vs. non-responding FGFR1 non-amplified lung models (P=0.029). The expression of FGFR1IIIc and FGFR3IIIc also displayed a positive trend with FGFR1-ECD.339-Fc response in the non-FGFR1 amplified lung subset in this experiment.

TABLE 5
Statistical analysis of FGF-related gene expression in relation to FGFR1-ECD.339-Fc
anti-tumor response in non-FGFR1 amplified lung xenograft models

| Gene | Ratio^{§} | P value^{†} | Gene | Ratio^{§} | P value^{†} |
|---|---|---|---|---|---|
| FGF2 | 3437 | 0.02857 | FGF8 | 0.3268 | 0.5338 |
| SPRY2 | 0.1395 | 0.05714 | FGF20 | 0.4803 | 0.6573 |
| FGFR3IIIc | 3.765 | 0.1375 | ELK4 | 1.019 | 0.6857 |
| DUSP5 | 0.3241 | 0.2 | FGFBP2 | 0.6526 | 0.6857 |
| FGFR1IIIc | 3.688 | 0.2343 | FLRT3 | 0.2211 | 0.6857 |
| FGF21 | 6.868 | 0.2454 | FGF11 | 2.039 | 0.7308 |
| FGFR2 | 8.793 | 0.2949 | FGF5 | 44.05 | 0.8294 |
| FGFR1 | 3.72 | 0.2949 | FGFR2IIIc | 2.029 | 0.8357 |
| FGF19 | 20.79 | 0.3094 | FGF1 | 1.45 | 0.8357 |
| FGFR1IIIb | 0.553 | 0.3429 | FGFR3 | 1.285 | 0.8357 |
| ELK3 | 0.5091 | 0.3429 | FGFR4 | 0.8265 | 0.8357 |
| SPRY4 | 0.3532 | 0.3429 | FGF10 | 0.4615 | 0.8357 |
| FGFBP1 | 0.1836 | 0.3429 | FGF17 | 0.4268 | 0.8357 |
| DUSP6 | 0.1254 | 0.3429 | ETV5 | 0.8563 | 0.8857 |
| DKK3 | 46.5 | 0.366 | FLRT2 | 0.828 | 0.8857 |
| FGF18 | 2.455 | 0.366 | FLRT1 | 0.8212 | 0.8857 |
| FGF22 | 1.373 | 0.3836 | PLAUR | 0.716 | 0.8857 |
| FGF2 | 30.92 | 0.4452 | FGFR3IIIb | 0.7137 | 0.8857 |
| VIM | 4.122 | 0.4452 | FGFR2b | 0.5752 | 0.8857 |
| ETV4 | 1.665 | 0.4452 | FGF16 | 1.786 | 0.9452 |
| FGFBP3 | 4.424 | 0.4857 | SPRY3 | 1.051 | 0.9452 |
| SOX9 | 0.3956 | 0.4857 | FGF9 | 2.07 | 1 |
| SERPINE1 | 0.3155 | 0.4857 | NCAM1 | 1.391 | 1 |
| SPRY1 | 0.1799 | 0.4857 | DUSP4 | 0.9031 | 1 |
| | | | FGF3 | 0.8571 | 1 |
| | | | FGF7 | 0.738 | 1 |

^{§}Gene expression ratio determined by median gene expression in FGFR1-ECD.339-Fc responders/median gene expression in non-responders
^{†}P-values are determined by a Mann-Whitney test of PCR gene expression in responders vs. non-responders for each gene using the non-FGFR1 amplified lung models in table 5.

It was examined if there was a correlation in gene expression amongst the significant gene markers identified for their association with FGFR1-ECD.339-Fc response in all models. The results of that analysis are shown in Table 6. In this experiment, there was a significant, positive correlation between the majority of the individual RNA markers identified as predictive for FGFR1-ECD.339-Fc xenograft response. For example, xenograft FGF2 RNA expression is positively correlated with FGFR3IIIc, FGFR1IIIc and FGFR1 expression (P<0.05); FGFR1 RNA expression is positively correlated with FGFR3IIIc, FGF2 and FGF18. The expression of ETV4 was not associated with other FGFR1-ECD.339-Fc responsive genes.

TABLE 6
Spearman correlation of gene expression markers predictive
of FGFR1-ECD.339-Fc efficacy in xenograft models

| Gene 1 | Gene 2 | Correlation | P-value^{§} |
|---|---|---|---|
| FGF18 | FGFR1 | 0.47 | 0.0083 |
| FGF18 | FGFR1IIIc | 0.57 | 0.0008 |
| FGF2 | FGFR3IIIc | 0.49 | 0.0139 |
| FGFR1 | FGFR3IIIc | 0.41 | 0.0244 |
| FGF2 | FGFR1IIIc | 0.43 | 0.0336 |
| FGF2 | FGFR1 | 0.39 | 0.0447 |

^{§}2-sided p-values approximated with a Monte Carlo simulation

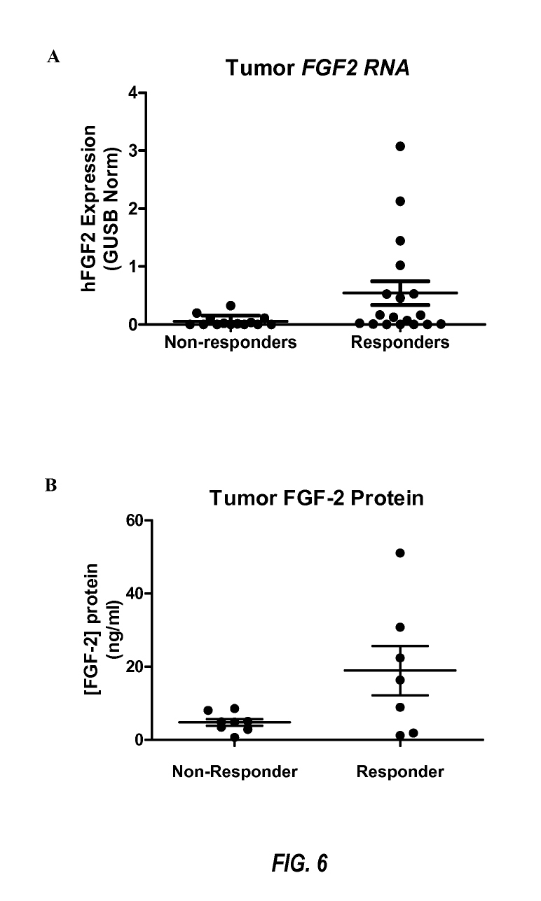

FIG. 6 shows (A) FGF2 mRNA (normalized to GUSB) and (B) FGF2 protein expression in FGFR1-ECD.339-Fc responder and non-responder xenografts. Expression of FGF2 (P=0.03569) was positively associated with FGFR1-ECD.339-Fc response. FGF2 displayed a high ratio (247.7-fold) of mRNA gene expression between FGFR1-ECD.339-Fc responder and non-responder xenografts. FGF2 protein levels were also confirmed to correlate with FGFR1-ECD.339-Fc response.

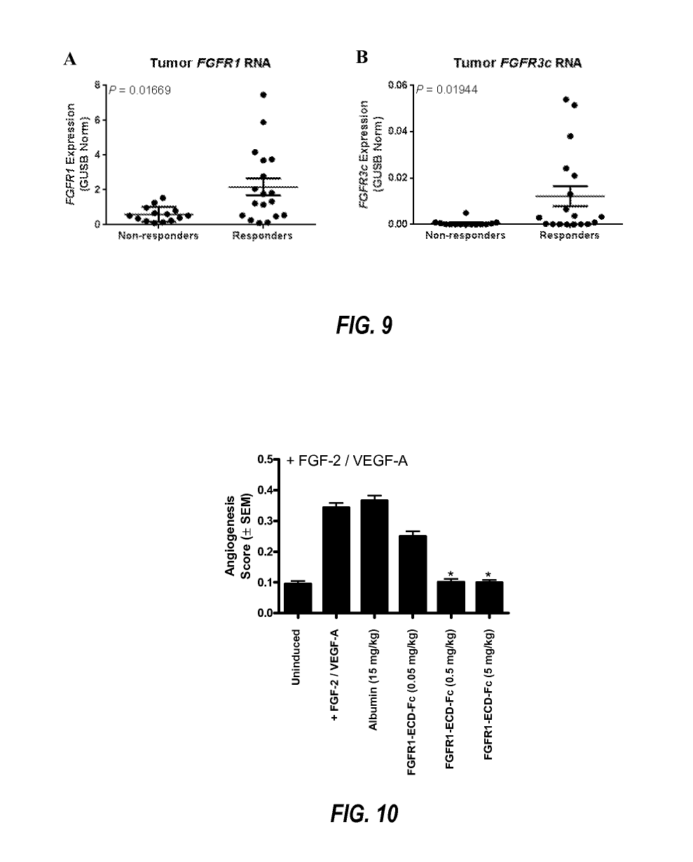

FIG. 9 shows (A) FGFR1 mRNA expression (normalized to GUSB) and (B) FGFR3IIIc mRNA expression (normalized to GUSB) in FGFR1-ECD.339-Fc responder and non-responder xenografts. Expression of FGFR1 (P=0.01669; FIG. 17a), and the FGFR1IIIc splice variant (P=0.0431; Table 4), was positively correlated with FGFR1-ECD.339-Fc anti-tumor activity. In addition to FGFR1, expression of the FGFR3IIIc receptor (P=0.01944, Table 4) was also positively correlated with FGFR1-ECD.339-Fc anti-tumor response (FIG. 5b), reflecting the overlap in FGF-ligand binding specificity between the c-splice isoforms of FGFR1 and FGFR3 receptors (see, e.g., Zhang, et al. J. Biol. Chem. 281, 15694-15700 (2006); Ornitz, et al. J. Biol. Chem. 271, 15292-15297 (1996)).

### Example 4

### Predictor of FGFR1-ECD.339-Fc Response

DKK3 mRNA expression was determined in a set of 25 xenografts using qRT-PCR. RNA was extracted from tumor xenografts grown in vivo using the RNAeasy® mini kit (Qiagen, Germany). Extracted RNA was treated with DNAse I prior to creating cDNA with random hexamer priming and reverse transcriptase using the QuantiTect Reverse Transcription Kit (Qiagen, Germany). Human DKK3 RNA expression was determined using QuantiTect Primer Assays (Qiagen, Germany) employing a human GUSB control reference QuantiTect Primer Assay (Qiagen, Germany). QuantiTect SYBR Green PCR Kits (Qiagen, Germany) were used to quantify mRNA expression levels using real-time qRT-PCR and an ABI Prism ViiA™ 7 Real-Time PCR System (Applied Biosystems, Foster City, Calif.). Relative gene expression quantification was calculated according to the comparative Ct method using human GUSB as a reference and commercial RNA controls (Stratagene, La Jolla, Calif.). Relative quantification was determined according to the formula: 2^{-(ΔCt sample-ΔCt calibrator)}.

The tumor xenografts used in this experiment are shown in Table 7. Also shown in Table 7 are the dosing schedule for FGFR1-ECD.339-Fc in a mouse xenograft model, the percent tumor growth inhibition (TGI (%)) and the statistical significance of the tumor growth inhibition (P Value).

TABLE 7
Panel of xenograft models with microarray data.

| | Xenograft | Cell line/ | Dosing | | | Dose | TGI | |
|---|---|---|---|---|---|---|---|---|
| Tumor type | model | PDX | route | Dose | | schedule | (%) | P Value |
| Colon | HCT116 | Cell Line | IP | 15 | mg/kg | BIW | 0% | ns |
| | Colo205 | Cell Line | IV | 5 | mg/kg | BIW | 38% | P < 0.001 |
| | Colo201 | Cell Line | IP | 15 | mg/kg | BIW | 0% | ns |
| Renal | A498 | Cell Line | IP | 15 | mg/kg | BIW | 7% | ns |
| | Caki-1 | Cell Line | IV | 10 | mg/kg | BIW | 81% | P < 0.001 |
| Lung | A549 | Cell Line | IP | 10 | mg/kg | BIW | 38% | P < 0.05 |
| | NCI-H460 | Cell Line | IP | 10 | mg/kg | BIW | 35% | P < 0.05 |
| | NCI-H226 | Cell Line | IP | 15 | mg/kg | 3x/w | 55% | P < 0.001 |
| | NCI-H520 | Cell Line | IP | 20 | mg/kg | BIW | 47% | P < 0.05 |
| | NCI-H1703 | Cell Line | IP | 15 | mg/kg | BIW | 31% | P < 0.05 |
| | NCI-H2126 | Cell Line | IP | 15 | mg/kg | BIW | 0% | ns |
| | NCI-H441 | Cell Line | IP | 15 | mg/kg | BIW | 0% | ns |
| | NCI-H358 | Cell Line | IP | 15 | mg/kg | BIW | 0% | ns |
| | NCI-H522 | Cell Line | IP | 10 | mg/kg | BIW | 42% | P < 0.05 |
| | NCI-H1581 | Cell Line | IP | 15 | mg/kg | BIW | 74% | P = 0.002 |
| | Calu-1 | Cell Line | IP | 15 | mg/kg | BIW | 0% | ns |
| Methothelioma | MSTO-211H | Cell Line | IP | 15 | mg/kg | BIW | 64% | P < 0.0001 |
| Glioblastoma | U-87 | Cell Line | IP | 15 | mg/kg | BIW | 0% | ns |
| | U-118 | Cell Line | IP | 15 | mg/kg | BIW | 36% | ns |
| | U-251 | Cell Line | IP | 15 | mg/kg | BIW | 48% | P = 0.0078 |
| Retinoblastoma | Y79 | Cell Line | IP | 10 | mg/kg | BIW | 21% | ns |
| Prostate | Du145 | Cell Line | IP | 0.15 | mg/kg | 3x/w | 31% | ns |
| Endometrial | HEC-1B | Cell Line | IP | 15 | mg/kg | BIW | 30% | P < 0.05 |
| Breast | MDA-MB-231 | Cell Line | IP | 15 | mg/kg | BIW | 0% | ns |
| | JIMT1 | Cell Line | IP | 1 | mg/kg | BIW | 28% | P < 0.05 |

Gene expression was then correlated to FGFR1-ECD.339-Fc response to determine RNA expression signatures positively and negatively correlated with anti-tumor activity. Expression of DKK3 mRNA was higher in tumors that were sensitive to FGFR1-ECD.339-Fc than in tumors that were not sensitive to FGFR1-ECD.339-Fc (P=0.0069).

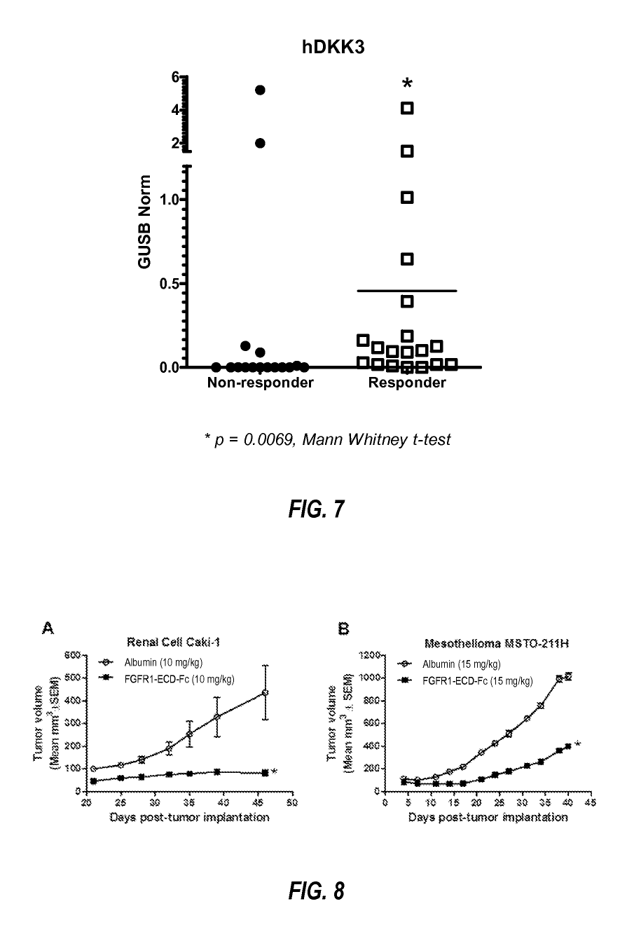

FIG. 7 shows DKK3 mRNA levels (normalized to GUSB) in FGFR1-ECD.339-Fc responder and non-responder xenografts. The horizontal line indicates the median expression level for that group.

### Example 5

### FGFR1-ECD.339-Fc Mediated Inhibition of FGF-2 and VEGF-A Induced Angiogenesis in a Matrigel Plug Assay

Recombinant human FGF-2 (final concentration 250 ng/ml; Peprotech) and/or recombinant human VEGF-A (final concentration 100 ng/ml; Peprotech) were added to matrigel (BD Biosciences, Franklin Lakes, N.J.) with sodium heparin (2 units/ml; Sigma). FGF-2 and/or VEGF-A containing matrigel plugs (one per animal) were implanted subcutaneously in the abdomen region of C57BL/6 mice (Charles River, Wilmington, Mass.). FGFR1-ECD.339-Fc was administered by tail vein injection on days 1, 4, and 7 post-matrigel implantation. On day 9, plugs were excised and processed for hematoxylin and eosin (H&E) staining. Digital images of the stained matrigel sections were generated using a Retiga 2000R digital camera (QImaging, Burnaby, BC). Image analysis was performed using Image-Pro Plus 5.1 (Media Cybernetics Inc., Silver Spring, Md.). Neovascularization was defined as the cellular response in the Matrigel plugs, consisting of newly formed blood vessels and migrated cells.

The results of that experiment are shown in FIG. 10. Administration of 5 mg/kg or higher FGFR1-ECD.339-Fc completely blocked in vivo angiogenesis induced by a matrigel plug impregnated with FGF-2. Administration of 15 or 45 mg/kg FGFR1-ECD.339-Fc also completely blocked in vivo angiogenesis in response to a matrigel plug impregnated with VEGF-A only or FGF-2 plus VEGF-A. Anti-angiogenic activity against VEGF induced angiogenesis in this model system may reflect inhibition of the synergistic activity between VEGF in the plug and murine-derived stromal FGFs since SPR analysis shows that FGFR1-ECD.339-Fc does not directly interact with VEGF-A.

To determine whether FGFR1-ECD.339-Fc blocks VEGF-induced proliferation of endothelial cells, HUVEC cells (Life Technologies, Grand Island, N.Y.) were seeded at a density of 4×10³ cells/well in basal media (Medium 200 (Life Technologies) with 2% heat inactivated FBS) and stimulated with either 10 ng/ml FGF2 (R&D Systems, Minneapolis, Minn.) or 15 ng/ml VEGF-A165 (R&D Systems, Minneapolis, Minn.) either in the presence of absence of 10 μg/ml FGFR1-ECD.339-Fc. HUVEC cell proliferation was determined 3 days post-stimulation using CellTiter-Glo® Luminescent Cell Viability Assay.

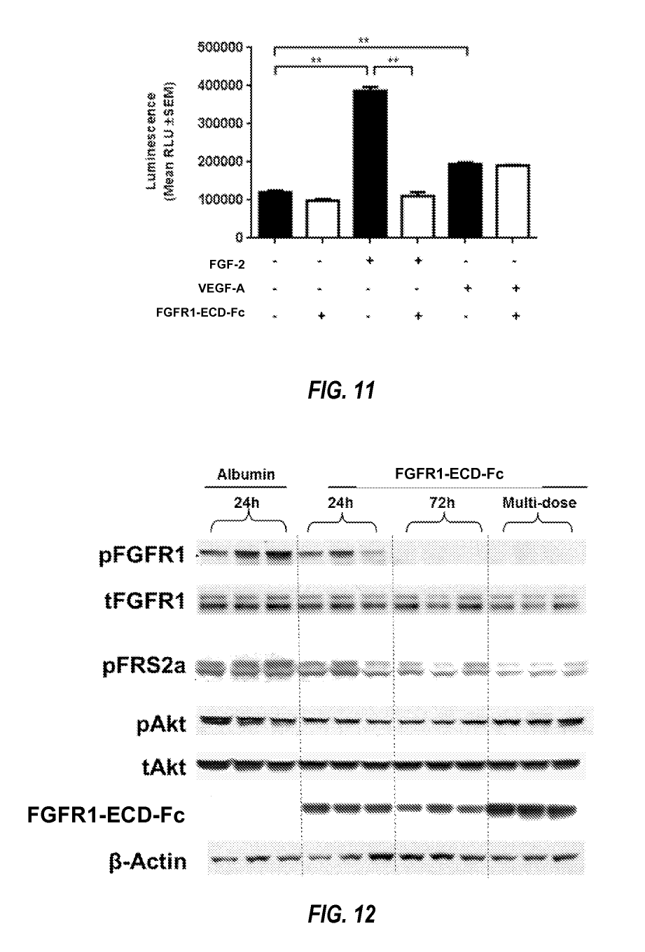

The results of that experiment are shown in FIG. 11. FGFR1-ECD.339-Fc did not block VEGF-induced proliferation of HUVECs, although it is capable of blocking FGF-2 induced HUVEC proliferation.

### Example 6

### FGFR1-ECD.339-Fc-Mediated Inhibition of FGFR1 Signaling in the JIMT-1 Breast Cancer Xenograft Model

Animals with established (200 mm3) human breast cancer JIMT-1 tumors were administered either a single (24 and 72 hour timepoints) or three times per week (multidose) i.p. dose(s) of FGFR1-ECD.339-Fc at 15 mg/kg. Tumor samples were collected at 24 and 72 hours post-dose for the single dose groups and 48 hours post the last dose in multi-dose group, snap-frozen in liquid nitrogen and lyzed in RIPA buffer (Sigma Aldrich, St Luis, Mo.). Tumor lysates were separated by SDS-PAGE and western blotting was performed using monoclonal antibodies FGFR1, pFGFR1, FRS2α, pFRS2α, Akt, pAkt, and βActin (Cell Signaling Technology, Inc). FGFR1-ECD.339-Fc was detected using anti-human Fc monoclonal antibody (Jackson Immuno Research).

The results of that experiment are shown in FIG. 12. FGFR1-ECD.339-Fc reduced levels of phosphorylated FGFR1 by 24 hours post-dose and completely abolished FGFR1 phosphorylation by 72 hours post-dose. Phosphorylated FRS and Akt levels were reduced 24 hours post-dose and further reduced two days later. Thus, FGFR1-ECD.339-Fc inhibited FGFR1 signaling in the JIMT-1 breast cancer xenograft model.

### Example 7

### Study to Evaluate Safety, Tolerability and Efficacy of FGFR1-ECD.339-Fc as a Single Agent in Humans

A Phase 1 first-time-in-human study (Study FP1039-001) has been completed. The study enrolled 39 subjects who received doses ranging from 0.6 mg/kg to 20.5 mg/kg FGFR1-ECD.339-Fc (calculated using EC=1.11 mL/mg*cm; equivalent to 0.5 mg/kg to 16 mg/kg of FGFR1-ECD.339-Fc calculated using EC=1.42 mL/mg*cm; see Table 1). A phase IB trial will be conducted to identify anticancer activity of FGFR1-ECD.339-Fc in subjects with malignancies with abnormal dependence on FGF pathway signaling. Activity will be explored in squamous non-small cell lung cancer (NSCLC) and in other malignancies where deregulated FGF pathway signaling, such as FGFR1 amplification, is present. It is anticipated that FGFR1-ECD.339-Fc monotherapy will demonstrate anti-tumor activity in the presence of deregulated FGF signaling pathway, specifically amplification or overexpression of FGF ligand(s) and/or receptor(s).

Primary objectives are to characterize the safety and tolerability of FGFR1-ECD.339-Fc as single agent, and to assess its efficacy and the overall response rate (ORR).

For selection of patients, inclusion criteria include histologically or cytologically confirmed diagnosis of advanced solid tumor with deregulated FGF pathway signaling, for which all lines of standard therapies have been exhausted or for which no standard treatment is available. Further, a ratio of FGFR1 gene copies to centromere 8 of greater than 2 will be required.

Squamous NSCLC subjects who have documented tumor progression (based on radiological imaging) after receiving two or more prior lines of systemic therapy (including platinum containing chemotherapy regimens) for Stage IV disease may be enrolled. See, e.g., TNM Classification of Malignant Tumors, 7^{th} edition, Sobin et al., Eds., 2009; Edge et al., 2010, Ann. Surg. Oncol., 17: 1471-1474.

Subjects with ER positive breast cancer having disease progression while on aromatase inhibitor therapy are allowed to continue aromatase inhibitor therapy, subjects with prostate cancer may continue to be treated with GnRH agonists or GnRH antagonists as clinically appropriate, and subjects with carcinoid cancer may continue treatment with octreotide.

Exclusion criteria include treatment with any anti-cancer therapy (for biological anti-cancer therapies see additional exclusion criteria herein) during the preceding 4 weeks or within 4 half-lives of the therapy, whichever is longer (except: anti-cancer hormonal treatment of prostate cancer, breast cancer or octreotide for treatment of carcinoid cancer), receipt of any biological therapy within 6 weeks of the first dose of FGFR1-ECD.339-Fc, conditions likely to increase the potential for abdominal perforation or fistula formation, symptomatic leptomeningeal or brain metastases or spinal cord compression.

Subjects will receive FGFR1-ECD.339-Fc administered as a 30-minute infusion once a week (Day 1, Day 8, and Day 15) at the starting dose of 20 mg/kg (calculated using EC=1.11 mL/mg*cm). In certain circumstances, subjects will receive FGFR1-ECD.339-Fc at the starting dose of 5 mg/kg, 10 mg/kg, or 15 mg/kg.

### Example 8

### Study to Evaluate Safety, Tolerability and Efficacy of FGFR1-ECD.339-Fc Plus Chemotherapy in Non-Small Cell Lung Cancer in Humans

### Arm A

The starting dose (Dose Level 0) and escalation/de-escalation schema for FGFR1-ECD.339-Fc in combination with paclitaxel+ carboplatin is presented in Table 8.

TABLE 8
FGFR1-ECD.339-Fc + Paclitaxel + Carboplatin

| | Dose of FGFR1-ECD.339-Fc | Paclitaxel + Carboplatin^{a} |
|---|---|---|
| Dose level | (weekly) | (once every 21 days) |
| Dose level −2 | 5 mg/kg | 135 mg/m² + AUC 4 |
| Dose level −1 | 5 mg/kg | 175 mg/m² + AUC 5 |
| Starting Dose | 5 mg/kg | 200 mg/m² + AUC 6 |
| Level 0 | | |
| Dose level 1 | 10 mg/kg | 200 mg/m² + AUC 6 |
| Dose level 2 | 20 mg/kg | 200 mg/m² + AUC 6 |

^{a}Carboplatin dose based on Calvert's formula

At least 12 subjects with stage IV squamous non-small cell lung cancer (according to TNM Classification of Malignant Tumors, 7^{th} edition, Sobin et al., Eds., 2009; Edge et al., 2010, Ann. Surg. Oncol., 17: 1471-1474); and up to 30 subjects will be enrolled at the target dose to further evaluate safety and efficacy. To avoid any undue delay in initiating systemic chemotherapy for subjects with newly diagnosed Stage IV disease, the first cycle of chemotherapy may be initiated while subjects are still in screening for the present study. The first dose of FGFR1-ECD.339-Fc should be given no later than Cycle 2 Day 1 of chemotherapy.

Subjects will receive FGFR1-ECD.339-Fc administered as a 30-minute infusion once a week (Day 1, Day 8, and Day 15) of each 21-day cycle at the dosages specified in Table 8. Following infusion of FGFR1-ECD.339-Fc, subjects should be observed for 1 hour prior to infusion of chemotherapeutic agents. If infusion reactions are noted, subjects should be treated with antiemetics, steroids, or antihistamines at the discretion of the investigator and premedication according to institutional standards before further infusions of FGFR1-ECD.339-Fc should be considered.

Subjects will receive pre-treatment for paclitaxel and carboplatin according to institutional standards. Paclitaxel according to the dose level being investigated as described in Table 8 will be administered intravenously over 3 hours (or according to local clinical standards) in a constant rate infusion on Day 1 of each 21 day treatment cycle immediately followed by i.v. carboplatin at a dose calculated for a target maximum AUC of AUC=6 as a 30 to 60 minute constant rate infusion (or according to local clinical standards). A total of 4 to 6 cycles of paclitaxel/carboplatin will be administered per local clinical practice.

Carboplatin will be dosed using the Calvert Formula (Calvert et al., J Clin Oncol. 1989; 11:1748-56). This approach uses a mathematical formula, which is based on a subject's pre-existing renal function or renal function and desired platelet nadir. Renal excretion is the major route of elimination for carboplatin. The formula calculates the dose based on a subject's glomerular filtration rate (GFR in mL/min) as measured by Cr-EDTA clearance and carboplatin target area under the concentration versus time curve (AUC in mg/ml·min). With the Calvert formula, the total dose of carboplatin is expressed in mg, NOT mg/m²:

Total Carboplatin Dose (mg)=(target AUC)×(GFR¹+25)

¹NOTE: The GFR used in the above Calvert formula to calculate AUC-based carboplatin dosing should not exceed 125 mL/min. Therefore, the maximum carboplatin dose (mg) equals the target AUC (mg/ml·min) multiplied by 150 mL/min.

Maximum Carboplatin Dose (mg)=target AUC (mg/ml·min)×(150 mL/min)

The maximum dose is based on a GFR estimate that is capped at 125 mL/min for patients with normal renal function. No higher estimated GFR values should be used.

For a target AUC=6, the maximum dose is 6×150=900 mg

For a target AUC=5, the maximum dose is 5×150=750 mg

For a target AUC=4, the maximum dose is 4×150=600 mg

The maximum target AUC explored in any cohort in this study is AUC=6. Therefore, using the Calvert formula above, the maximum carboplatin dose in mg should not exceed 900 mg.

The Cockroft-Gault formula (see below) can be used to calculate the creatinine clearance (CLCR), which can be substituted for the GFR in the Calvert formula.

$$\begin{matrix}ClCr ( mL \text{/} min ) = \frac{Q \times ( 140 - age [ yr ] ) \times idea l body {weight [ kg ]}^{*}}{72 \times serum creatinine [ mg \text{/} dL )} \\ Q = 0.85 for females \\ Q = 1.0 for males \\ OR \\ ClCr ( mL \text{/} min ) = \frac{K \times ( 140 - age [ yr ] ) \times ideal body {weight [ kg ]}^{*}}{Serum creatinine [ umol / L ]} \\ K = 1.0 for females \\ K = 1.23 for males\end{matrix}$$

### * Calculation of Ideal Body Weight Using the Devine Formula [Devine, 1974]

Ideal body weight:
Males=50.0 kg+(2.3 kg×each inch over 5 feet) or 50.0 kg+(0.906 kg×each cm over 152.4 cm)
Females=45.5 kg+(2.3 kg×each inch over 5 feet) or 45.5 kg+(0.906 kg×each cm over 152.4 cm)
Example: Male, actual body weight=90.0 kg, height=68 inches
   Ideal body weight=50.0+(2.3)(68.60)=68.4 kg.
      This subject's actual body weight is >30% over ideal body weight. Therefore, in this case, the subject's ideal body weight of 68.4 kg should be used in calculating estimated CrCl.

Additional information, packaging, preparation and administration information can be found in the prescribing information for carboplatin (Paraplatin USPI).

### Arm B

The starting dose (Dose Level 0) and escalation/de-escalation schema for FGFR1-ECD.339-Fc in combination with docetaxel is presented in Table 9.

TABLE 9
FGFR1-ECD.339-Fc + Docetaxel

| | Dose of FGFR1-ECD.339-Fc | Docetaxel |
|---|---|---|
| Dose level | (weekly) | (once every 21 days) |
| Dose level −2 | 5 mg/kg | 40 mg/m² |
| Dose level −1 | 5 mg/kg | 55 mg/m² |
| Starting Dose | 5 mg/kg | 75 mg/m² |
| Level 0 | | |
| Dose level 1 | 10 mg/kg | 75 mg/m² |
| Dose level 2 | 20 mg/kg | 75 mg/m² |

At least 12 subjects with stage IV squamous non-small cell lung cancer (according to TNM Classification of Malignant Tumors, 7^{th} edition, Sobin et al., Eds., 2009; Edge et al., 2010, Ann. Surg. Oncol., 17: 1471-1474); and up to 30 subject will be enrolled at the target dose to further evaluate safety and efficacy.

Subjects will receive FGFR1-ECD.339-Fc administered as a 30-minute infusion once a week (Day 1, Day 8, and Day 15) of each 21-day cycle at the dosages specified in Table 9. Following infusion of FGFR1-ECD.339-Fc, subjects should be observed for 1 hour prior to infusion of chemotherapeutic agents. If infusion reactions are noted, subjects should be treated with antiemetics, steroids, or antihistamines at the discretion of the investigator and premedication according to institutional standards before further infusions of FGFR1-ECD.339-Fc should be considered.

Subjects in Arm B will receive pre-treatment for docetaxel according to institutional standards. Docetaxel will be administered according to the dose level being explored as described in Table 9 as an i.v. infusion over 1 hour (or according to local clinical standards) on Day 1 of each 21 day cycle. The subject is treated until progression or until the subject has been determined to have received maximum benefit. For additional formulation, packaging, preparation and administration information, please refer to the product labeling (e.g. US package insert or product monograph).

### TABLE OF SEQUENCES

Table 10 lists certain sequences discussed herein. FGFR1 sequences are shown without the signal peptide, unless otherwise indicated.

TABLE 10
Sequences and Descriptions

| SEQ ID NO | Description | Sequence |
|---|---|---|
| 1 | Full-length human | MWSWKCLLFW AVLVTATLCT ARPSPTLPEQ AQPWGAPVEV |
| | FGFR1 ECD (with signal | ESFLVHPGDL LQLRCRLRDD VQSINWLRDG VQLAESNRTR |
| | peptide); SP-hFGFR1- | ITGEEVEVQD SVPADSGLYA CVTSSPSGSD TTYFSVNVSD |
| | ECD.353 | ALPSSEDDDD DDDSSSEEKE TDNTKPNPVA PYWTSPEKME |
| | | KKLHAVPAAK TVKFKCPSSG TPNPTLRWLK NGKEFKPDHR |
| | | IGGYKVRYAT WSIIMDSVVP SDKGNYTCIV ENEYGSINHT |
| | | YQLDVVERSP HRPILQAGLP ANKTVALGSN VEFMCKVYSD |
| | | PQPHIQWLKH IEVNGSKIGP DNLPYVQILK TAGVNTIDKE |
| | | MEVLHLRNVS FEDAGEYTCL AGNSIGLSHH SAWLIVLEAL |
| | | EERPAVMTSP LYLE |
| 2 | Full-length human | RPSPTLPEQ AQPWGAPVEV ESFLVHPGDL LQLRCRLRDD |
| | FGFR1 ECD (without | VQSINWLRDG VQLAESNRTR IIGEEVEVQD SVPADSGLYA |
| | signal peptide); | CVISSPSGSD TTYFSVNVSD ALPSSEDDDD DDDSSSEEKE |
| | hFGFR1-ECD.353 | TDNTKPNPVA PYWTSPEKME KKLHAVPAAK IVKFKCPSSG |
| | | TPNPTLRWLK NGKEFKPDHR IGGYKVRYAI WSIIMDSVVP |
| | | SDKGNYTCIV ENEYGSINHI YQLDVVERSP HRPILQAGLP |
| | | ANKIVALGSN VEFMCKVYSD PQPHIQWLKH IEVNGSKIGP |
| | | DNLPYVQILK TAGVNTIDKE MEVLHLRNVS FEDAGEYTCL |
| | | AGNSIGLSHH SAWLIVLEAL EERPAVMTSP LYLE |
| 3 | SP-hFGFR1-ECD.339 | MWSWKCLLFW AVLVTATLCT ARPSPTLPEQ AQPWGAPVEV |
| | | ESFLVHPGDL LQLRCRLRDD VQSINWLRDG VQLAESNRTR |
| | | IIGEEVEVQD SVPADSGLYA CVISSPSGSD TTYFSVNVSD |
| | | ALPSSEDDDD DDDSSSEEKE TDNTKPNPVA PYWTSPEKME |
| | | KKLHAVPAAK IVKFKCPSSG TPNPTLRWLK NGKEFKPDHR |
| | | IGGYKVRYAI WSIIMDSVVP SDKGNYTCIV ENEYGSINHI |
| | | YQLDVVERSP HRPILQAGLP ANKIVALGSN VEFMCKVYSD |
| | | PQPHIQWLKH IEVNGSKIGP DNLPYVQILK TAGVNTIDKE |
| | | MEVLHLRNVS FEDAGEYTCL AGNSIGLSHH SAWLIVLEAL |
| 4 | hFGFR1-ECD.339 | RPSPILPEQ AQPWGAPVEV ESFLVHPGDL LQLRCRLRDD |
| | | VQSINWLRDG VQLAESNRTR IIGEEVEVQD SVPADSGLYA |
| | | CVISSPSGSD TTYFSVNVSD ALPSSEDDDD DDDSSSEEKE |
| | | TDNTKPNPVA PYWTSPEKME KKLHAVPAAK IVKFKCPSSG |
| | | TPNPTLRWLK NGKEFKPDHR IGGYKVRYAI WSIIMDSVVP |
| | | SDKGNYTCIV ENEYGSINHI YQLDVVERSP HRPILQAGLP |
| | | ANKIVALGSN VEFMCKVYSD PQPHIQWLKH IEVNGSKIGP |
| | | DNLPYVQILK TAGVNTIDKE MEVLHLRNVS FEDAGEYTCL |
| | | AGNSIGLSHH SAWLIVLEAL |
| 5 | SP-hFGFR1-ECD.339-Fc | MWSWKCLLFW AVLVTATLCT ARPSPTLPEQ AQPWGAPVEV |
| | | ESFLVHPGDL LQLRCRLRDD VQSINWLRDG VQLAESNRTR |
| | | IIGEEVEVQD SVPADSGLYA CVISSPSGSD TTYFSVNVSD |
| | | ALPSSEDDDD DDDSSSEEKE TDNTKPNPVA PYWTSPEKME |
| | | KKLHAVPAAK IVKFKCPSSG TPNPTLRWLK NGKEFKPDHR |
| | | IGGYKVRYAI WSIIMDSVVP SDKGNYTCIV ENEYGSINHI |
| | | YQLDVVERSP HRPILQAGLP ANKIVALGSN VEFMCKVYSD |
| | | PQPHIQWLKH IEVNGSKIGP DNLPYVQILK TAGVNTIDKE |
| | | MEVLHLRNVS FEDAGEYTCL AGNSIGLSHH SAWLIVLEAL |
| | | EPKSSDKIHI CPPCPAPELL GGPSVFLFPP KPKDTLMISR |
| | | TPEVTCVVVD VSHEDPEVKF NWYVDGVEVH NAKIKPREEQ |
| | | YNSIYRVVSV LIVLHQDWLN GKEYKCKVSN KALPAPIEKI |
| | | ISKAKGQPRE PQVYILPPSR DELIKNQVSL ICLVKGFYPS |
| | | DIAVEWESNG QPENNYKTTP PVLDSDGSFF LYSKLTVDKS |
| | | RWQQGNVFSC SVMHEALHNH YTQKSLSLSP GK |
| 6 | hFGFR1-ECD.339-Fc | RPSPTLPEQ AQPWGAPVEV ESFLVHPGDL LQLRCRLRDD |
| | | VQSINWLRDG VQLAESNRTR ITGEEVEVQD SVPADSGLYA |
| | | CVISSPSGSD TTYFSVNVSD ALPSSEDDDD DDDSSSEEKE |
| | | TDNTKPNPVA PYWTSPEKME KKLHAVPAAK TVKFKCPSSG |
| | | TPNPTLRWLK NGKEFKPDHR IGGYKVRYAT WSIIMDSVVP |
| | | SDKGNYTCIV ENEYGSINHT YQLDVVERSP HRPILQAGLP |
| | | ANKTVALGSN VEFMCKVYSD PQPHIQWLKH IEVNGSKIGP |
| | | DNLPYVQILK TAGVNTIDKE MEVLHLRNVS FEDAGEYTCL |
| | | AGNSIGLSHH SAWLTVLEAL EPKSSDKTHT CPPCPAPELL |
| | | GGPSVFLFPP KPKDTLMISR TPEVTCVVVD VSHEDPEVKF |
| | | NWYVDGVEVH NAKTKPREEQ YNSTYRVVSV LTVLHQDWLN |
| | | GKEYKCKVSN KALPAPIEKT ISKAKGQPRE PQVYTLPPSR |
| | | DELTKNQVSL TCLVKGFYPS DIAVEWESNG QPENNYKTTP |
| | | PVLDSDGSFF LYSKLTVDKS RWQQGNVFSC SVMHEALHNH |
| | | YTQKSLSLSP GK |
| 7 | hFGFR1 signal peptide | MWSWKCLLFWAVLVTATLCTA |
| 8 | Fc C237S | EPKSSDKTHT CPPCPAPELL GGPSVFLFPP KPKDTLMISR |
| | | TPEVTCVVVD VSHEDPEVKF NWYVDGVEVH NAKTKPREEQ |
| | | YNSTYRVVSV LTVLHQDWLN GKEYKCKVSN KALPAPIEKT |
| | | ISKAKGQPRE PQVYTLPPSR DELTKNQVSL TCLVKGFYPS |
| | | DIAVEWESNG QPENNYKTTP PVLDSDGSFF LYSKLTVDKS |
| | | RWQQGNVFSC SVMHEALHNH YTQKSLSLSP GK |
| 9 | Exemplary Fc #1 | ERKCCVECPP CPAPPVAGPS VFLFPPKPKD TLMISRTPEV |
| | | TCVVVDVSHE DPEVQFNWYV DGVEVHNAKT KPREEQFNST |
| | | FRVVSVLTVV HQDWLNGKEY KCKVSNKGLP APIEKTISKT |
| | | KGQPREPQVY TLPPSREEMT KNQVSLTCLV KGFYPSDIAV |
| | | EWESNGQPEN NYKTTPPMLD SDGSFFLYSK LTVDKSRWQQ |
| | | GNVFSCSVMH EALHNHYTQK SLSLSPGK |
| 10 | Exemplary Fc #2 | ESKYGPPCPS CPAPEFLGGP SVFLFPPKPK DTLMISRTPE |
| | | VTCVVVDVSQ EDPEVQFNWY VDGVEVHNAK TKPREEQFNS |
| | | TYRVVSVLTV LHQDWLNGKE YKCKVSNKGL PSSIEKTISK |
| | | AKGQPREPQV YTLPPSQEEM TKNQVSLTCL VKGFYPSDIA |
| | | VEWESNGQPE NNYKTTPPVL DSDGSFFLYS RLTVDKSRWQ |
| | | EGNVFSCSVM HEALHNHYTQ KSLSLSLGK |

1. A method of treating breast cancer in a subject comprising administering a therapeutically effective amount of an FGFR1 ECD or an FGFR1 ECD fusion molecule to the subject, wherein prior to administration at least a portion of the cells of the breast cancer were determined to have FGFR1 gene amplification, FGFR1 overexpression, FGFR3 overexpression, or FGF2 overexpression; and to be estrogen receptor (ER) positive, progesterone (PR) positive, or ER positive and PR positive.

2. The method of claim 1, wherein prior to administration the cancer was determined to be HER2 positive.

3. The method of claim 2, wherein prior to administration the cancer was determined to be p95HER2 positive.

4. The method of any one of claims 1 to 3, wherein the subject has previously been administered, or is currently being administered, trastuzumab or lapatinib.

5. The method of claim 1, wherein prior to administration the cancer was determined to be HER2 negative.

6. The method of any one of the preceding claims, wherein the breast cancer is ER positive.

7. The method of any one of the preceding claims, wherein the breast cancer is PR positive.

8. The method of any one of the preceding claims, wherein the subject has previously been administered, or is currently being administered, an aromatase inhibitor.

9. A method of treating prostate cancer in a subject comprising administering a therapeutically effective amount of an FGFR1 ECD or an FGFR1 ECD fusion molecule to the subject, wherein prior to administration at least a portion of the cells of the prostate cancer were determined to have FGFR1 gene amplification, FGFR1 overexpression, FGFR3 overexpression, or FGF2 overexpression, and wherein the subject has previously been administered, or is currently being administered, a therapeutic agent selected from a gonadotropin releasing hormone (GnRH) agonist, a GnRH antagonist, an androgen receptor (AR) inhibitor, and a 17-hydroxylase inhibitor.

10. The method of claim 9, wherein the subject has previously been administered, or is currently being administered, a gonadotropin releasing hormone (GnRH) agonist or a GnRH antagonist.

11. The method of claim 10, wherein the subject has previously been administered, or is currently being administered, a GnRH antagonist.

12. A method of treating carcinoid cancer in a subject comprising administering a therapeutically effective amount of an FGFR1 ECD or an FGFR1 ECD fusion molecule to the subject, wherein prior to administration at least a portion of the cells of the carcinoid cancer were determined to have FGFR1 gene amplification, FGFR1 overexpression, FGFR3 overexpression, or FGF2 overexpression, and wherein the subject has previously been administered, or is currently being administered, octreotide.

13. A method of treating ovarian cancer in a subject comprising administering a therapeutically effective amount of an FGFR1 ECD or an FGFR1 ECD fusion molecule to the subject, wherein prior to administration at least a portion of the cells of the ovarian cancer were determined to have FGFR1 gene amplification, FGFR1 overexpression, FGFR3 overexpression, or FGF2 overexpression, and wherein the subject has previously been administered, or is currently being administered, tamoxifen or an aromatase inhibitor.

14. The method of claim 13, wherein the ovarian cancer is estrogen receptor (ER) positive, progesterone (PR) positive, or ER positive and PR positive.

15. A method of treating lung cancer in a subject comprising administering at least 5 mg/kg of an FGFR1 ECD or an FGFR1 ECD fusion molecule and at least 135 mg/m² paclitaxel and at least AUC 4 carboplatin to the subject.

16. The method of claim 15, wherein the method comprises administering from 135 mg/m² paclitaxel to 200 mg/m² paclitaxel, at least 175 mg/m² paclitaxel, from 175 mg/m² paclitaxel to 200 mg/m² paclitaxel, or 200 mg/m² paclitaxel.

17. The method of claim 15 or claim 16, wherein the method comprises administering from AUC 4 carboplatin to AUC 6 carboplatin, at least AUC 5 carboplatin, from AUC 5 carboplatin to AUC 6 carboplatin, or AUC 6 carboplatin.

18. A method of treating lung cancer in a subject comprising administering at least 5 mg/kg of an FGFR1 ECD or an FGFR1 ECD fusion molecule and at least 40 mg/m² docetaxel.

19. The method of claim 18, wherein the method comprises administering from 40 mg/m² docetaxel to 75 mg/m² docetaxel, at least 55 mg/m² docetaxel, from 55 mg/m² docetaxel to 75 mg/m² docetaxel, or 75 mg/m² docetaxel.

20. The method of any one of claims 15 to 19, wherein the method comprises administering from 5 mg/kg to 20 mg/kg of an FGFR1 ECD or an FGFR1 ECD fusion molecule, at least 10 mg/kg of an FGFR1 ECD or an FGFR1 ECD fusion molecule, from 10 mg/kg to 20 mg/kg of an FGFR1 ECD or an FGFR1 ECD fusion molecule, at least 15 mg/kg of an FGFR1 ECD or an FGFR1 ECD fusion molecule, from 15 mg/kg to 20 mg/kg of an FGFR1 ECD or an FGFR1 ECD fusion molecule, or 20 mg/kg of an FGFR1 ECD or an FGFR1 ECD fusion molecule.

21. The method of any one of claims 15 to 20, wherein the lung cancer is non-small cell lung cancer.

22. The method of claim 21, wherein the non-small cell lung cancer is squamous non-small cell lung cancer.

23. The method of any one of the preceding claims, wherein at least a portion of the cells of the cancer have an FGFR1 gene amplification.

24. The method of claim 23, wherein at least a portion of the cells of the cancer having FGFR1 gene amplification comprise at least three copies of the FGFR1 gene.

25. The method of claim 24, wherein at least a portion of the cells of the cancer having FGFR1 gene amplification comprise at least four, at least five, at least six, or at least eight copies of the FGFR1 gene.

26. The method of claim 23, wherein at least a portion of the cells of the cancer having an FGFR1 gene amplification have a ratio of FGFR1 gene to chromosome 8 centromere of at least 1.5.

27. The method of claim 26, wherein the ratio of FGFR1 gene to chromosome 8 centromere is at least 2, at least 2.5, at least 3, at least 3.5, or at least 4.

28. The method of claim 26, wherein the ratio of FGFR1 gene to chromosome 8 centromere is greater than 2.

29. The method of any one of claims 23 to 28, wherein FGFR1 gene amplification was determined by a method selected from fluorescence in situ hybridization, array comparative genomic hybridization, DNA microarray, spectral karyotyping, quantitative PCR, southern blotting, or sequencing.

30. The method of any one of the preceding claims, wherein at least a portion of the cells of the cancer have FGFR1 overexpression.

31. The method of claim 30, wherein FGFR1 is FGFR1IIIc.

32. The method of any one of the preceding claims, wherein at least a portion of the cells of the cancer have FGF2 overexpression.

33. The method of any one of the preceding claims, wherein at least a portion of the cells of the cancer have FGFR3 overexpression.

34. The method of claim 33, wherein FGFR3 is FGFR3IIIc.

35. The method of any one of the preceding claims, wherein at least a portion of the cells of the cancer overexpress at least one, at least two, or three markers selected from DKK3, FGF18, and ETV4.

36. The method of any one of the preceding claims, wherein at least a portion of the cells of the cancer overexpress at least one or two markers selected from DKK3 and FGF18.

37. The method of any one of the preceding claims, wherein at least a portion of the cells of the cancer overexpress ETV4.

38. The method of any one of claims 30 to 37, wherein the cancer does not have an FGFR1 gene amplification.

39. The method of any one of claims 30 to 38, wherein the overexpression is protein overexpression.

40. The method of claim 39, wherein protein overexpression is determined using immunohistochemistry.

41. The method of any one of claims 30 to 38, wherein the overexpression is mRNA overexpression.

42. The method of claim 41, wherein mRNA overexpression is determined using quantitative RT-PCR.

43. The method of any one of the preceding claims, wherein the method comprises administering an FGFR1 ECD.

44. The method of claim 43, wherein the FGFR1 ECD comprises an amino acid sequence selected from SEQ ID NOs: 1 to 4.

45. The method of any one of claims 1 to 42, wherein the method comprises administering an FGFR1 ECD fusion molecule.

46. The method of claim 45, wherein the FGFR1 ECD fusion molecule comprises an FGFR1 ECD and a fusion partner, and wherein the fusion partner is Fc.

47. The method of claim 46, wherein the FGFR1 ECD fusion molecule comprises a sequence selected from SEQ ID NO: 5 and SEQ ID NO: 6.
